(19) United States
(12) Patent Application Publication
(10) Pub. No.: US 2026/0071221 A1
(43) Pub. Date: Mar. 12, 2026

(54) Fibroblast Growth Factor (FGF)21 Inhibition for Treatment of Cardiac Hypertrophy

(71) Applicant: University of Cincinnati, Cincinnati, OH (US)

(72) Inventors: Konstantinos Drosatos, Cincinnati, OH (US); Md Sobuj Mia, Cincinnati, OH (US)

(73) Assignee: University of Cincinnati, Cincinnati, OH (US)

(21) Appl. No.: 19/268,918

(22) Filed: Jul. 14, 2025

Related U.S. Application Data
(60) Provisional application No. 63/670,208, filed on Jul. 12, 2024.
(60) Provisional application No. 63/755,774, filed on Feb. 7, 2025.

(51) Int. Cl. C12N 15/113

(52) U.S. Cl. CPC C12N 15/1136; C12N 2310/11

(57) ABSTRACT

A method of treating a subject with a disease where the disease is selected from the group consisting of hypertension, cardiac hypertrophy, cardiomyocyte enlargement, myocardial fibrosis, liver fibrosis, and cardiac fibrosis is provided. The method involves administering to the subject an effective amount of a fibroblast growth factor (FGF) 21 inhibitor.

### CROSS-REFERENCE TO RELATED APPLICATIONS

This application claims priority to, and the benefit of the filing date of, U.S. Provisional Application No. 63/670,208 filed Jul. 12, 2024, and U.S. Provisional Application No. 63/755,774 filed Feb. 7, 2025, the disclosures of which are incorporated by reference herein in their entirety.

### FIELD OF THE INVENTION

This invention relates generally to treatments for cardiac hypertrophy.

### BACKGROUND OF THE INVENTION

Heart failure (HF) still constitutes a major cause of mortality and hospital admissions despite decades of efforts for the improvement of applied treatments. In many cases, HF is accompanied by left ventricular hypertrophy (LVH). LVH is an early adaptive mechanism in response to increased heart workload, which can progress to detrimental cardiac complications associated with high cardiovascular morbidity and mortality. The increase in myocardial wall thickness allows the heart to counterbalance pressure-induced wall stress that may be caused from physiological stressors such as exercise or from pathological events such as aortic stenosis or uncontrolled hypertension. In pathological states, the hypertrophic response is accompanied by fibrosis, inflammation, arrhythmia, and eventually systolic and diastolic myocardial dysfunction. Ongoing studies aim to understand the underlying mechanisms and develop clinically tractable methods for prevention. While various intracellular signaling pathways that are involved in cardiomyocyte (CM) enlargement and LVH are known, the systemic mechanisms that ignite the onset of the disease remain partially characterized.

Fibroblast growth factor (FGF) 21 is an endocrine hormone that regulates systemic energy homoeostasis. It is mainly produced in the liver but skeletal muscle, white adipose tissue, brown adipose tissue, and the pancreas also contribute to the systemic FGF21 levels. Various studies suggest that the heart can also serve as a source of FGF21. FGF21 signals through the β-Klotho/FGF receptor (FGFR)1c complex. Pharmacological administration of FGF21 in animal models of diabetes and obesity, as well as treatment of obese humans with FGF21 analogs normalized circulating glucose and lipids and lowered body weight and hepatic fat.

While the metabolic benefits of FGF21 have been translated to clinical applications, the effects of prolonged FGF21 signaling to the heart remain controversial. Stimulation of cardiac FGF21 production has been described as an adaptive response to physiological stress. On the other hand, increased FGF21 signaling has been associated with cardiac dysfunction in end-stage HF patients, as well as with diastolic dysfunction and higher mortality in patients with HF with preserved ejection fraction (HFpEF), hypertension, dilated cardiomyopathy, coronary artery disease, acute myocardial infarction, atrial fibrillation, and Type 2 diabetes (T2D). The discrepancy between beneficial metabolic effects and potential detrimental consequences of FGF21 signaling in cardiac function indicates a need to better understand how FGF21 is involved in HF that is induced with chronic stress.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, a method of treating a subject with a disease where the disease is selected from the group consisting of hypertension, cardiac hypertrophy, cardiomyocyte enlargement, myocardial fibrosis, liver fibrosis, and cardiac fibrosis is provided. The method involves administering to the subject an effective amount of a fibroblast growth factor (FGF) 21 inhibitor.

In one embodiment, the cardiac hypertrophy is left ventricular hypertrophy. In another embodiment, the FGF21 inhibitor is a cardiomyocyte FGF21 inhibitor. In one embodiment, the FGF21 inhibitor is a hepatocyte FGF21 inhibitor.

In another embodiment, the administration of inhibition of FGF21 inhibitor is cardioprotective against transverse aortic constriction induced heart failure. In one embodiment, the FGF21 inhibitor is a hepatocyte FGF21 inhibitor. In another embodiment, the FGF21 inhibitor further comprises a pharmaceutically acceptable excipient.

### BRIEF DESCRIPTION OF THE DRAWINGS

The objects and advantages of the disclosed invention will be further appreciated in light of the following detailed descriptions and drawings in which:

FIGS. 1A-1K are a series of images showing that hepatic and cardiac FGF21 expression is increased in humans and mice with increased afterload. Cardiac hypertrophy, cardiac function, and fibrosis-related gene expression changes during progression of cardiac hypertrophy.

FIGS. 2A and 2B are a pair of images showing that changes in cardiac transcriptome, metabolome and lipidome occur as soon as 3 days post-TAC and continue throughout the progression of cardiac hypertrophy.

FIGS. 3A-3P are a series of images showing that genetic ablation of hepatocyte FGF21 lowers cardiac FGF21 expression and attenuates cardiac hypertrophy and dysfunction.

FIGS. 4A-4L are a series of images showing that genetic deletion of cardiomyocyte FGF21 prevents cardiac hypertrophy independent of systemic FGF21 levels.

FIGS. 5A-5K are a series of images showing that the anti-hypertrophic effects of hepatocyte or cardiomyocyte FGF21 suppression share partial overlap in transcriptome, metabolome and lipidome profile changes; oxytocin signaling is a common activated pathway.

FIGS. 6A-6J are a series of images showing that AAV-mediated overexpression of cardiac FGF21 negates the cardioprotective effect of hepatocyte-specific FGF21 ablation in pressure overload.

FIGS. 7A-7K are a series of images showing that antisense oligonucleotide mediated inhibition of FGF21 holds therapeutic potential against LVH from increased afterload.

FIG. 8 is a schematic showing a graphical abstract of the invention.

FIGS. 9A-9G are a series of images showing a pressure gradient and comparison of FGF21 abundance in male and female mice.

FIGS. 10A-10F are a series of images showing a KEGG pathway analysis of differentially regulated genes at 3 days, 2 weeks, and 8 weeks post-TAC.

FIGS. 11A-11G are a series of images showing that hemodynamic congestion in the liver caused by pIVCL or TAC induces the expression of hepatic FGF21 and mechano-sensor-related genes.

FIGS. 12A-12G are a series of images showing that FGF21^{f1/f1}, Alb-Cre^{+/-} and αMHC-Cre^{+/-} mice respond similarly to pressure overload.

FIGS. 13A-13G are a series of images showing that both male and female mice with hepatocyte-specific FGF21 deletion exhibit similar responses to pressure overload-induced FGF21 expression, cardiac hypertrophy and fibrosis.

FIGS. 14A-14Q are a series of images showing that hepatocyte-specific Fgf21 ablation attenuates cardiac fibrosis- and inflammation-related gene expression, and pro-hypertrophic cellular signaling pathways.

FIGS. 15A-15L are a series of images showing that treatment of AC16 cells (human cardiomyocyte cell line) with recombinant FGF21 activates endogenous FGF21 expression and ERK signaling. Cardiac expression of FGF receptors and co-receptor β-klotho in mice with TAC and AC16 cells treated with recombinant FGF21.

FIGS. 16A-16C are a series of images showing that hepatocyte-specific FGF21 ablation reduced pressure overload-induced cardiac PPARα, Sirtuin1 and pAKT/tAKT protein abundance.

FIGS. 17A-17L are a series of images showing that FGF21 expression in cardiomyocytes, liver and white adipose tissue of mice with cardiomyocyte-specific FGF21 ablation, both male and female mice, exhibit similar FGF21 expression, as well as cardiac hypertrophy- and fibrosis-related gene expression in response to pressure overload.

FIGS. 18A-18O are a series of images showing that cardiomyocyte-specific FGF21 ablation attenuates pressure overload-induced cardiac and systemic inflammation as well as pro-hypertrophic cellular signaling pathways.

FIGS. 19A-19D are a series of images showing that KEGG analysis of transcriptome for downregulated pathways in HEP-FGF21−/− and CM-FGF21−/− mice 8 weeks post-TAC.

FIGS. 20A-20D are a series of images showing that KEGG analysis of transcriptome for upregulated pathways in HEP-FGF21−/− and CM-FGF21−/− mice 8 weeks post-TAC.

FIGS. 21A and 21B are a pair of images showing that expression of cardiac oxytocin during cardiac hypertrophic growth in mouse and human hearts.

FIGS. 22A-22D are a series of images showing that AAV-mediated cardiomyocyte-specific overexpression of FGF21 negates the protective effect of hepatocyte FGF21 ablation against cardiac hypertrophy.

FIGS. 23A-23E are a series of images showing selection and validation of ASO-FGF21.

FIGS. 24A-24H are a series of images showing ASO-FGF21 reduced gene expression related to cardiac fibrosis, inflammation and heart failure in mice with TAC.

### DEFINITIONS

As used herein, the term “pharmaceutically acceptable” or “pharmacologically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Moreover, for animal (e.g., human) administration, it will be understood that compositions should meet sterility, pyrogenicity, general safety and purity standards as required by the FDA Office of Biological Standards.

As used herein, the term “pharmaceutically acceptable excipient” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, disintegrating agents, binders, sweetening agents, flavoring agents, perfuming agents, protease inhibitors, plasticizers, emulsifiers, stabilizing agents, viscosity increasing agents, film forming agents, solubilizing agents, surfactants, preservative and antioxidants can also be present in the formulation. The terms such as “excipient”, “carrier”, “pharmaceutically acceptable excipient” or the like are used interchangeably herein.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The primary role of the heart is to pump blood efficiently and ensure adequate oxygen and nutrients supply to peripheral organs. When preload or afterload is increased, the heart and individual cardiomyocytes undergo hypertrophy. The enlargement of cardiomyocytes and the left ventricle initially enhances contractility, thus increasing cardiac efficiency, a process known as physiological hypertrophy that is induced by normal stress, such as exercise. However, prolonged thickening of the left ventricular wall constitutes pathological hypertrophy, which is mainly associated with aortic stenosis or uncontrolled hypertension. Some of the main hallmarks that distinguish pathological from physiological hypertrophy are fibrosis and inflammation, which predispose to ischemia, arrhythmias, and eventually HF and increased mortality. Various studies have suggested multiple cellular signaling pathways that are involved in cardiomyocyte enlargement and cardiac hypertrophy. However, the systemic mechanisms that cause or contribute to cardiac wall hypertrophy remain partially characterized.

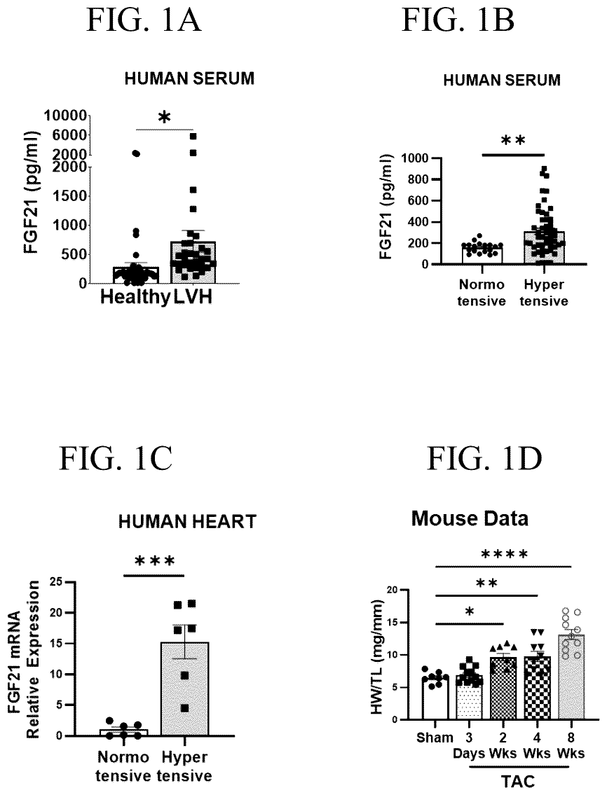

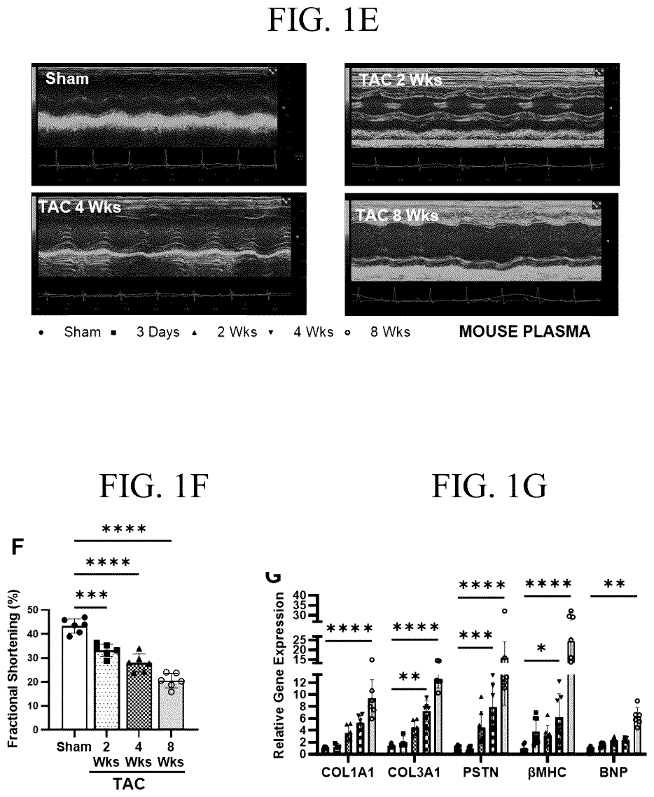

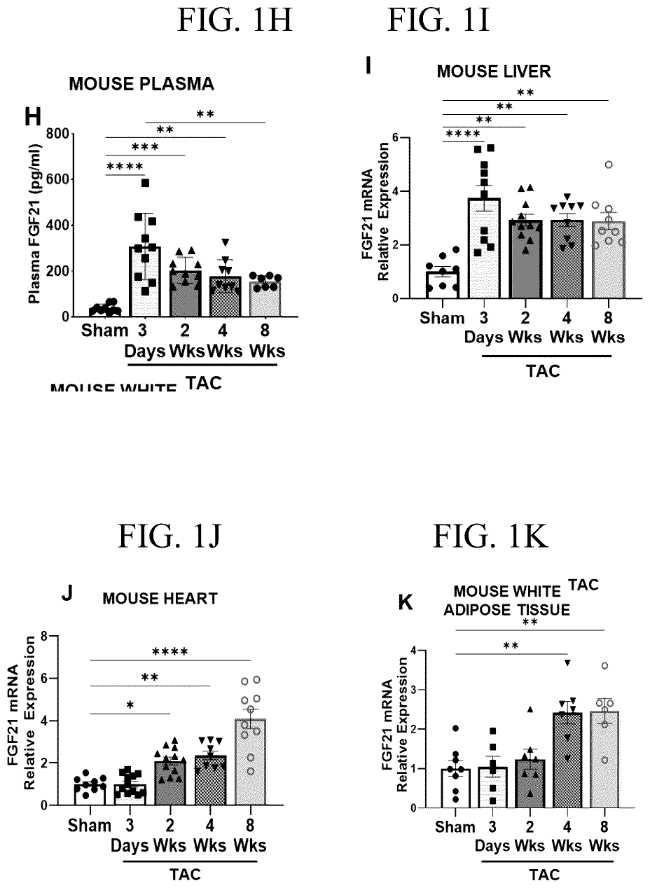

Based on findings in myocardial samples of patients with advanced HFrEF25, who show increased FGF21 staining, and observations presented herein of higher serum FGF21 levels in patients with LVH or hypertension (FIGS. 1A, 1B), the potential involvement of FGF21 in LVH pathophysiology and FGF21-based therapeutic applications was explored. The results presented herein in a mouse model of pressure overload indicate a novel pro-hypertrophic signaling pathway which entails liver-heart crosstalk that is spearheaded by FGF21. Interestingly, hepatic FGF21 induces FGF21 expression in cardiomyocytes that serves as the main cause of LVH in an autocrine fashion.

FGF21 is an endocrine metabolic hormone that is induced by nutritional and physiological stress. It is mainly produced in the liver but also in other tissues including the heart. Increased plasma FGF21 levels have been reported in obese humans and animal models. Although this could be perceived as a component of a pathological mechanism, a study that suggested FGF21 resistance in obese mice concluded that FGF21 production in obesity is a protective mechanism. However, while the transient beneficial effects of FGF21 are obvious, it remains unclear whether chronic FGF21 signaling is beneficial or aggravating for obesity. Thus, the effects of FGF21 in body weight have been controversial.

A similar controversy exists regarding the effects of FGF21 in cardiac physiology. However, as shown herein, our results show that inhibition of hepatocyte FGF21 is cardioprotective against transverse aortic constriction (“TAC”) induced HF. Moreover, inhibition of cardiomyocyte FGF21 is also protective despite high levels of hepatic FGF21 expression and serum FGF21 levels. In accordance with our findings associating FGF21 with cardiac toxicity, an FGF21 analog that has been used in humans increased systolic and diastolic blood pressure.

A recent human study also revealed strong association of FGF21 with hypertrophic cardiomyopathy. Hypertension studies showed higher circulating FGF21 levels in mice, as well as in humans, which we also observed (FIG. 1). Plasma FGF21 is also increased in a genetic mouse model of hypertrophic cardiomyopathy (heterozygous αMHC403/+ for the human disease-causing mutation Arg403Gln). Accordingly, mice with global Fgf21 deletion are protected from pregnancy-induced cardiomyopathy. Altogether, these findings suggest that prolonged FGF21 signaling may elicit aggravating consequences in heart function, at least in certain types of cardiomyopathy.

In our attempt to address these controversies mostly between clinical observations and preclinical studies with global FGF21−/− mice, we performed experiments with tissue-specific FGF21 knockout mouse models. Our studies revealed that induction of FGF21 expression in cardiomyocytes is critical for the onset of pathological hypertrophy. To explore if the lack of cardiac phenotype in mice that overexpress cardiomyocyte FGF21 may be because cardiac stress was not applied, we performed combined AAV-mediated FGF21 cDNA transfer in cardiomyocytes and TAC in HEP-FGF21−/− mice that were protected from LVH. Our findings confirmed that cardiomyocyte FGF21 induction itself negates the protective effect of hepatocyte FGF21 ablation against pressure overload-driven LVH.

In addition to our finding that chronic FGF21 upregulation aggravates cardiac function, we also identified a time lag of approximately 2 weeks between the increase in hepatic and plasma FGF21 levels and the stimulation of cardiomyocyte FGF21 expression that leads to pathological hypertrophy and HF, accompanied by fibrosis and inflammation. Interestingly, we also observed upregulation of FGF21 expression in the WAT 4 weeks post-TAC, which occurs 2 weeks after the onset of LVH and cardiac dysfunction. Thus, although WAT FGF21 upregulation is not among the early events that lead to LVH, it may constitute a response of the WAT to increased catecholamine secretion, which target 0-adrenergic receptors in WAT and other tissues, thus leading to higher FGF21 expression. We have shown that suppression of fibroblast activation prevents TAC-induced cardiac hypertrophy. Thus, cardiac hypertrophy with pressure overload seems to require both fibroblast activation and induction of cardiomyocyte FGF21 expression. It remains to be elucidated whether fibroblast activation precedes or follows cardiomyocyte FGF21 upregulation.

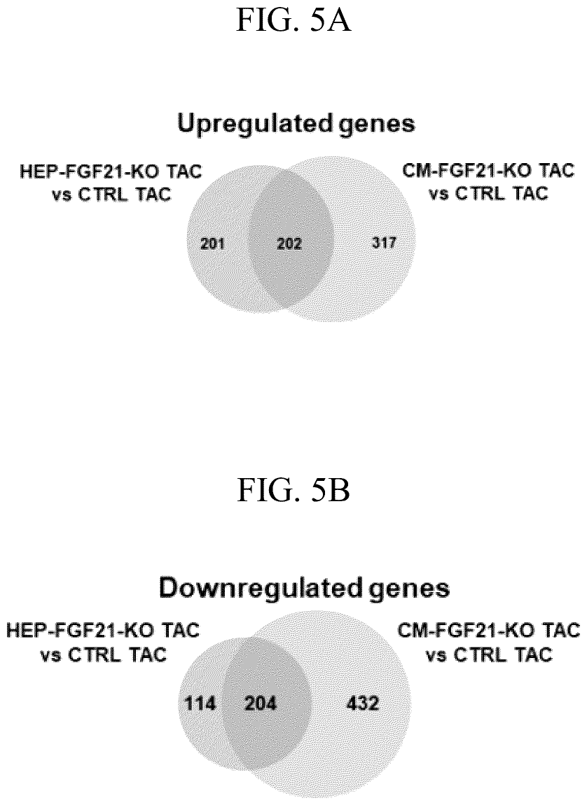

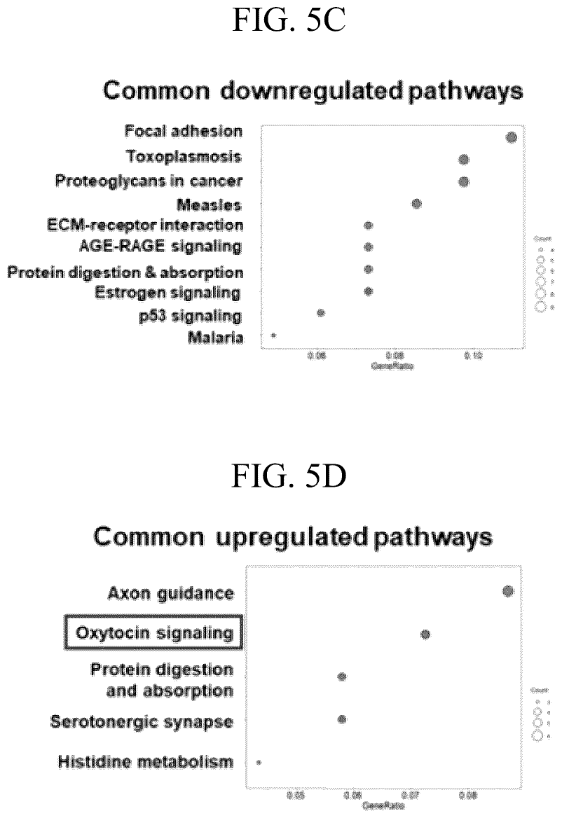

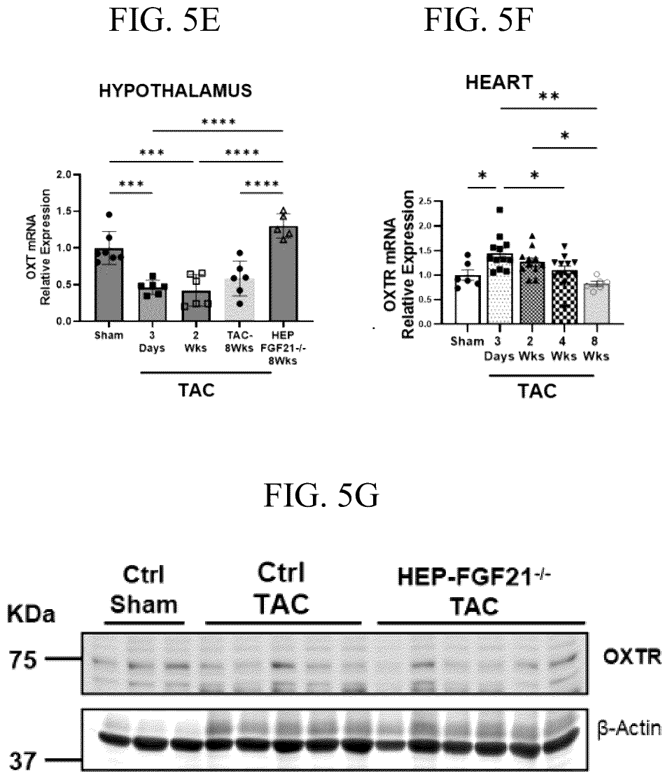

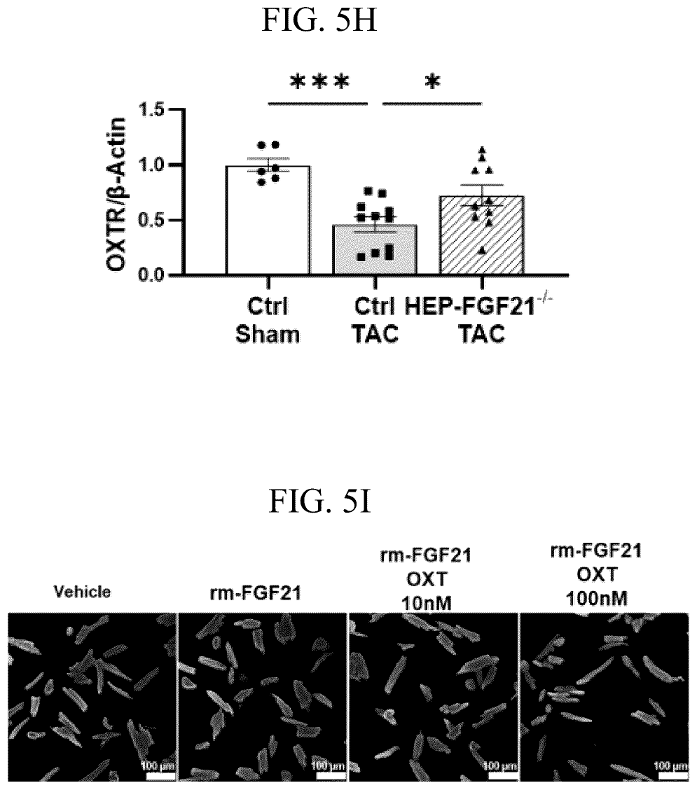

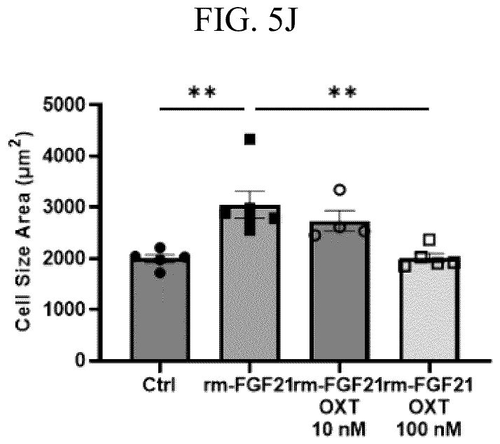

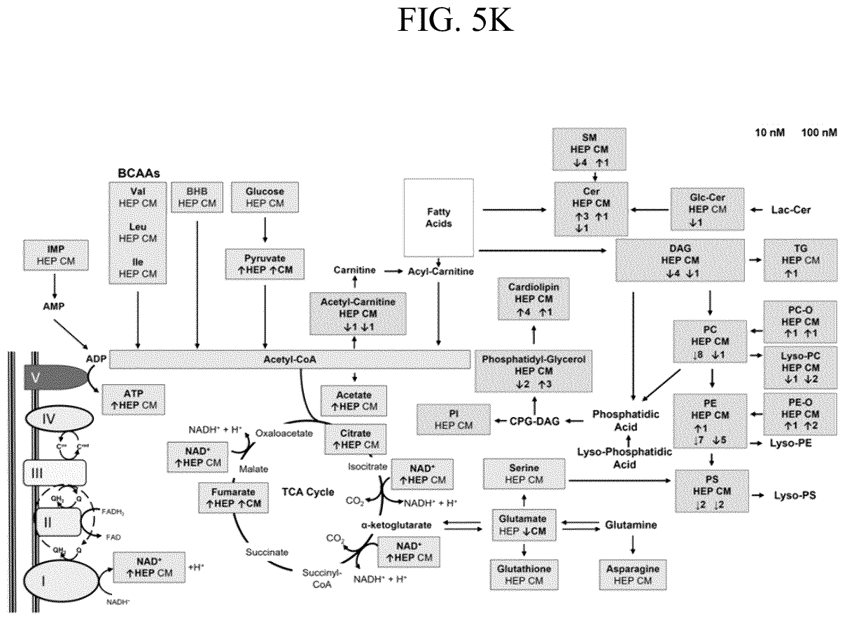

Moreover, as it has been shown that non-cardiac organs or cells may mediate either the effects of FGF21 or cardiac hypertrophy, such as the central nervous system or inflammatory cells, the involvement of other systems in modulating the pro-hypertrophic effect of FGF21 remains to be explored. One of the pathways that were induced with the protective inhibition of either hepatic or cardiomyocyte FGF21 was oxytocin signaling. Oxytocin is a neuropeptide that can be produced either by the hypothalamus or the heart and has been associated with cardiac protection in various types of cardiomyopathy. Specifically, oxytocin prevents CM hypertrophy as shown by others and our new data, LVH and fibrosis in ovariectomized rats. It also attenuates parasympathetic activation that protects from myocardial ischemia-induced complications, oxidative stress, inflammation and atrial natriuretic peptide release that lowers cardiac afterload. Oxytocin exerts its signaling effects via binding on oxytocin receptor (OXT-R), which is a Gq-protein coupled receptor. OXT-R is present in many tissues including the heart of humans and rodents. In our study, oxytocin expression was not affected in the heart. Although FGF21 increases oxytocin expression in hypothalamus, we show that prolonged systemic FGF21 upregulation leads to its suppression, likely via a negative feedback loop mechanism. Our in vitro studies demonstrate that exposing cardiomyocytes to oxytocin prevents FGF21-induced enlargement of cardiac cell size (FIGS. 5A-5K). This finding is aligned with our in vivo RNA-seq analysis, which shows that oxytocin signaling components are restored following the genetic deletion of FGF21 in either the liver or heart during pressure overload. These results suggest that FGF21 signaling attenuates oxytocin signaling in the heart. Future studies remain to elucidate whether competition between FGF21 and oxytocin signaling may account for the gradual progression to pathological LVH.

The importance of identifying potential involvement of FGF21 in LVH is signified by the use of recombinant FGF21, stable FGF21 analogs and receptor agonists in pre-clinical and Phase I and II clinical studies for the treatment of obesity, insulin resistance, hyperglycemia, severe hypertriglyceridemia, atherosclerosis, and hepatic steatosis. We have found that such therapies need to be applied with extra caution to people of increased risk for HF.

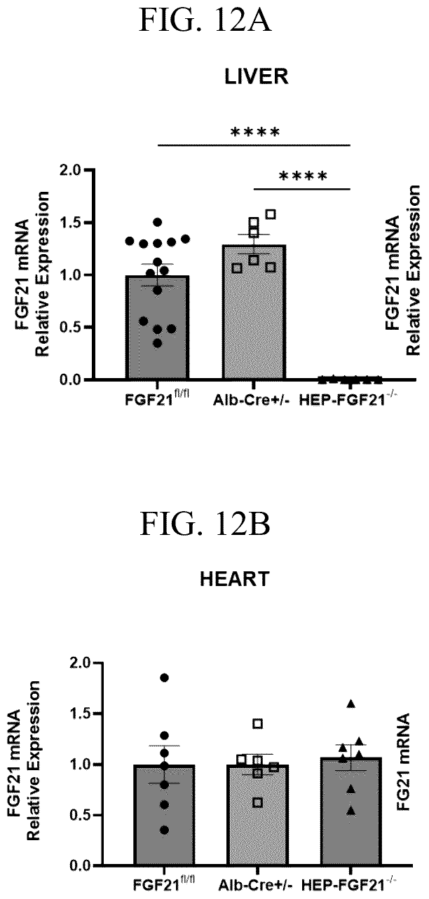

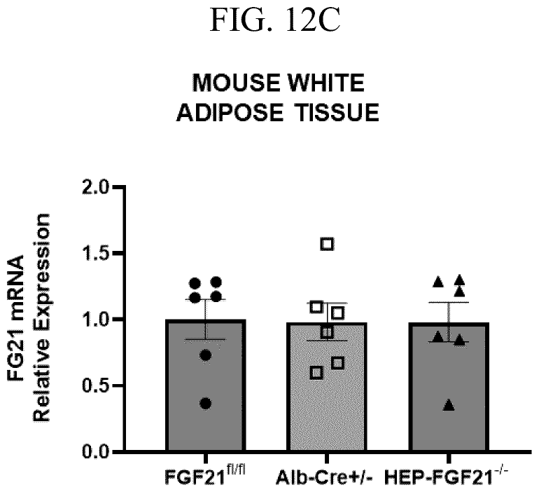

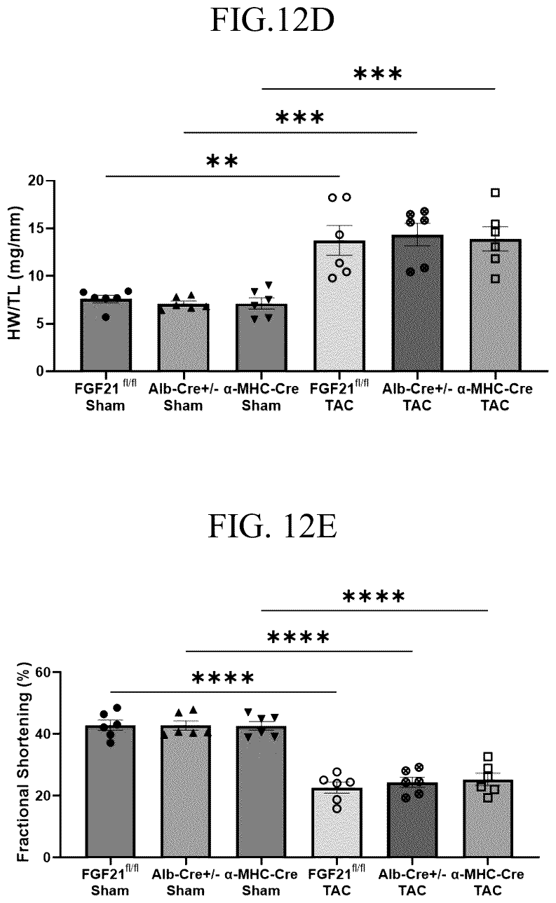

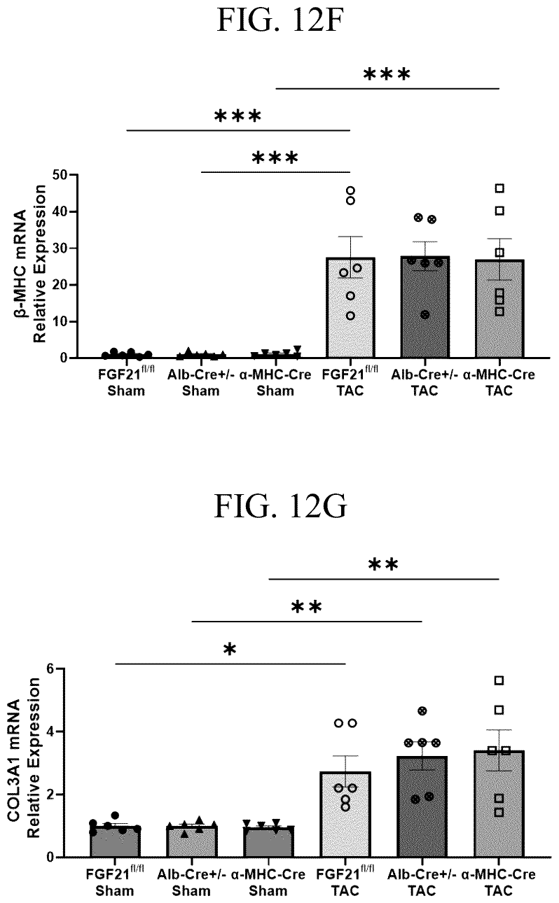

To secure sufficient biological replicates in our experimental groups (at least 6/group), some of our control mouse cohorts include a combined population of floxed mice and tissue-specific (either hepatic or cardiac depending on the experiment) Cre-expressing mice. However, our pilot studies (FIGS. 12D-12G) showed that Cre-expressing and non-expressing mice exhibit similar phenotypes.

The present invention involves a hepato-cardiac signaling pathway that accounts for left ventricular hypertrophy (LVH) and HF that are induced with pressure overload. This pathway involves FGF21 production in the liver, which targets the heart leading to cardiomyocyte FGF21 production that drives hypertrophy via a previously unknown mechanism that seems to interfere with the cardioprotective signaling of hypothalamus-derived oxytocin. The importance of elucidating potential involvement of FGF21 in causing HF is signified by the use of recombinant FGF21, stable FGF21 analogs and receptor agonists in pre-clinical and Phase I and II clinical studies for the treatment of obesity, insulin resistance, hyperglycemia, severe hypertriglyceridemia, atherosclerosis, and hepatic steatosis. We have found that FGF21 therapies need to be applied with extra caution particularly for individuals that are at higher risk for developing cardiac complications.

The results presented herein show that pressure overload leads to LVH via a combined endocrine-autocrine hepato-cardiac mechanism that implicates FGF21 as the main signaling molecule. This mechanism involves an early response of the liver to cardiac pressure overload that leads to higher expression and release of FGF21. Hepatic FGF21 eventually orchestrates the onset of LVH via stimulation of FGF21 expression in cardiomyocytes, which acts on an autocrine fashion. Simultaneously, FGF21 seems to suppress cardioprotective signaling to the heart by lowering oxytocin expression in the hypothalamus. Interventions that inhibit FGF21 signaling hold therapeutic capacity for the treatment of LVH that is caused by increased afterload.

### EXAMPLES

### Example 1

Blood and cardiac FGF21 levels are increased in human patients and mice with cardiac hypertrophy or increased afterload. Recently, it was shown that circulating levels of FGF21 are substantially elevated in end-stage heart failure patients, as well as that this change coincided with higher FGF21 deposition in myocardium samples. To explore further whether FGF21 is involved in HF, we assessed serum FGF21 levels in humans with LVH (Table 1) or hypertension (Table 2). Neither of the patient groups have developed systolic dysfunction, which occurs with cardiac hypertrophy. Our analyses identified higher serum FGF21 levels in both patient groups compared to control groups without LVH (FIG. 1A) or those without hypertension (FIG. 1), respectively. To investigate if observations that correlated high serum FGF21 levels with elevated myocardial FGF21 abundance in patients with end-stage heart failure apply in patients with increased afterload, we measured FGF21 expression in heart tissue samples from male and female individuals with systemic hypertension (Table 3) (samples were obtained from the National Disease Research Interchange). Our analysis showed significantly higher FGF21 mRNA levels (15-fold) in hearts of hypertensive individuals compared to normotensives (FIG. 1C). Thus, humans with increased afterload or LVH have higher serum and cardiac FGF21 content even prior to the development of systolic dysfunction.

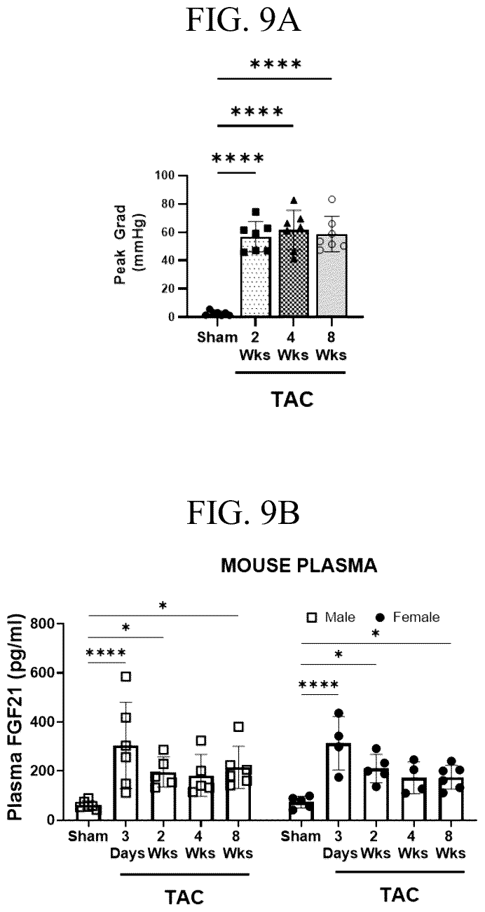

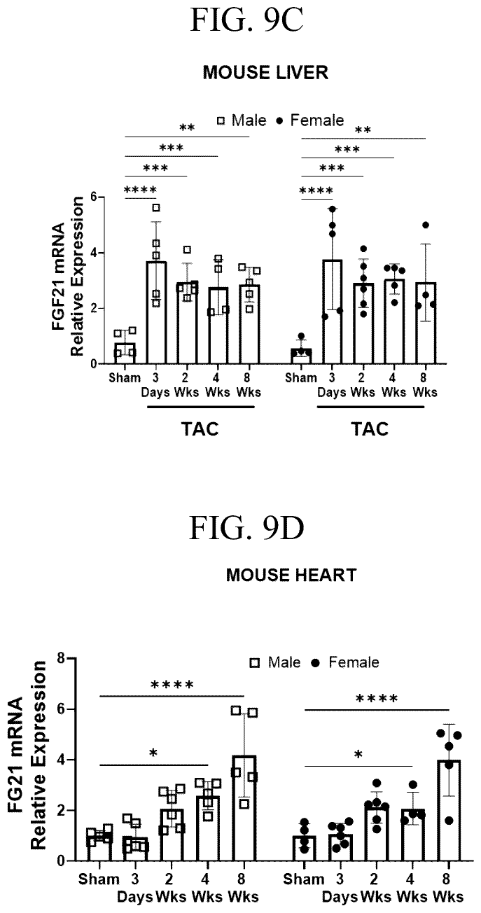

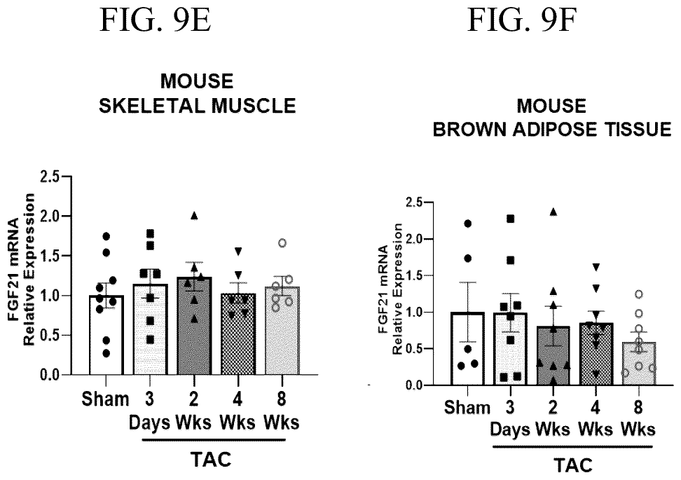

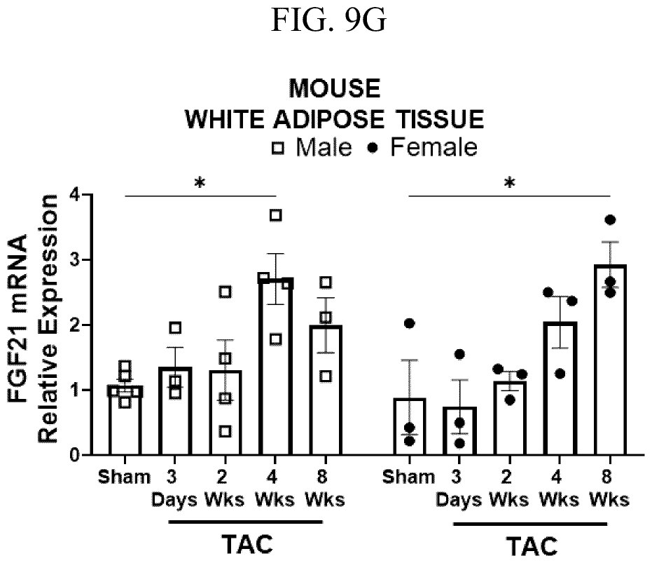

These observations motivated us to test whether elevation in serum FGF21 constitutes an adaptive or maladaptive response to increased cardiac afterload. To this end, we investigated how FGF21 production changes over the course of the development of LVH in a mouse model of increased LV afterload (transverse aortic constriction; TAC). We applied TAC for 3 days, 2 weeks, 4 weeks and 8 weeks (FIG. 9A for TAC-induced pressure gradient data for sham, 2 weeks, 4 weeks and 8 weeks) in wild-type mice, which induced cardiac hypertrophy (FIG. 1D) and cardiac dysfunction (FIGS. 1E-F, Table 4 and 5) and increased cardiac expression of the fibrosis markers, Collagen type I alpha 1 (COL1A1), COL3A1, Periostin (PSTN), and the heart failure markers β-myosin heavy chain (β-MHC) and B-type Natriuretic Peptide (BNP) (FIG. 1G). Pressure overload increased plasma FGF21 levels (FIG. 1H) in both male and female mice (FIG. 9B) that coincided with higher expression of FGF21 in the liver (FIG. 1I) in both sexes (FIG. 9C). In accordance with our findings in humans (FIG. 1C), both male and female mice with pressure overload had higher cardiac FGF21 expression (FIG. 1J, FIG. 9D). On the other hand, FGF21 expression did not increase either in the skeletal muscle (FIG. 9E) or in the brown adipose tissue (FIG. 9F), although it was increased in white adipose tissue (WAT) in both sexes (FIG. 1K and FIG. 9G). Thus, pressure overload is associated with higher systemic FGF21 levels and increased expression in the liver, WAT, and the heart.

### Example 2

To explore whether upregulation of hepatic and cardiac FGF21 expression constitutes a protective adaptive response to pressure overload or a cause for the development of cardiac hypertrophy and systolic dysfunction, we characterized the temporal profile of FGF21 expression in relation to the onset of cardiac dysfunction. Hepatic FGF21 expression (FIG. 1I) and plasma FGF21 levels (FIG. 1H) increased as early as 3 days post-TAC and remained elevated (˜6-fold) up to 8 weeks in both males and females (FIGS. 9B and 9C). Although hepatic and plasma FGF21 upregulation constitutes an early response to pressure overload, the increase in cardiac FGF21 expression occurred with a delay (2 weeks post-TAC) (FIG. 1J, FIG. 9D). Notably, elevation of cardiac FGF21 expression coincided with the onset of cardiac hypertrophy (FIG. 1D), cardiac dysfunction (FIGS. 1E and 1F, Table 4 and 5), and the upregulation of cardiac fibrosis-related and heart failure-related (BNP, β-MHC) gene expression (FIG. 1G), which has been associated with pathological cardiac hypertrophy49. Interestingly, the increase of FGF21 expression in WAT was observed even later (4 weeks post-TAC), after cardiac dysfunction and hypertrophy had occurred (FIG. 1K). Conclusively, pressure overload instantly stimulates FGF21 expression in the liver and release of FGF21 in the circulation, which are followed later by induction of cardiac FGF21 that coincides with the onset of LVH and left ventricular dysfunction and upregulation of WAT FGF21 that follows cardiac complications.

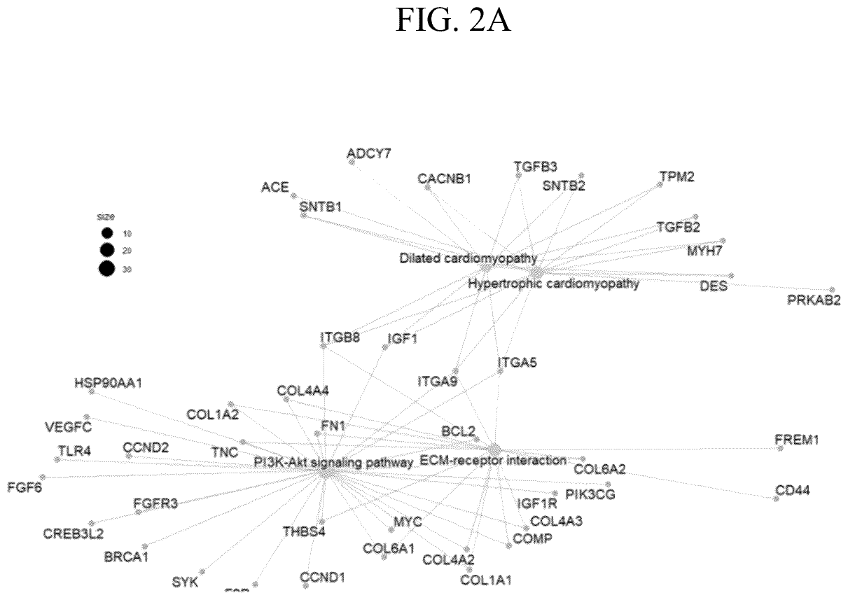

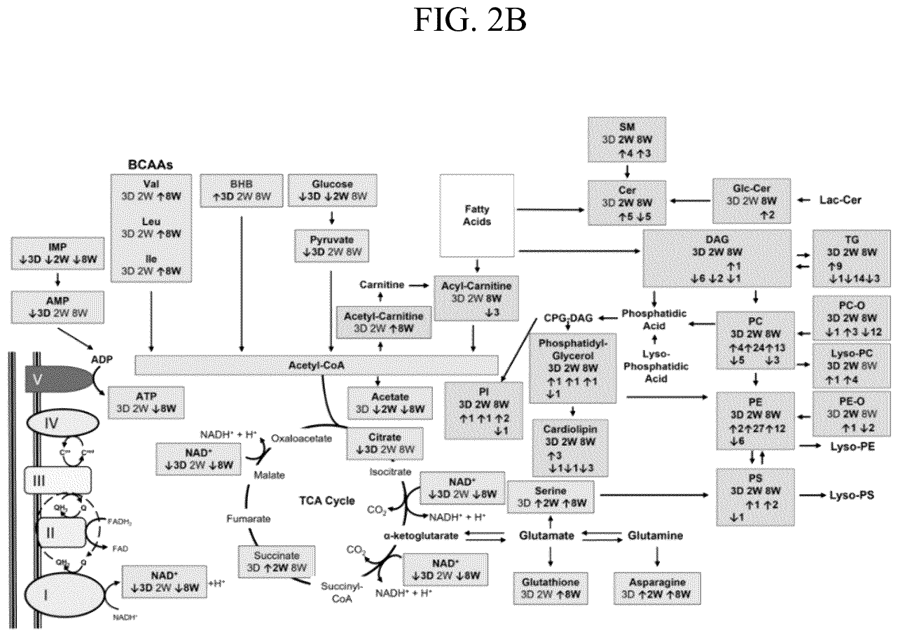

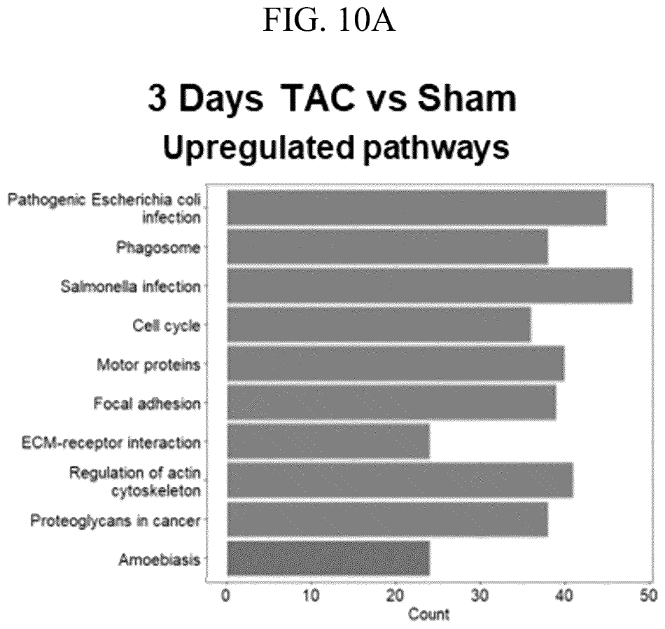

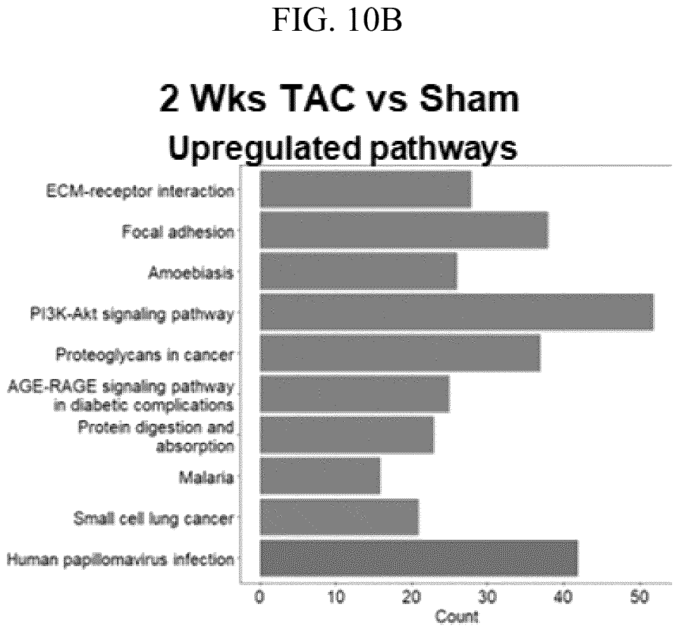

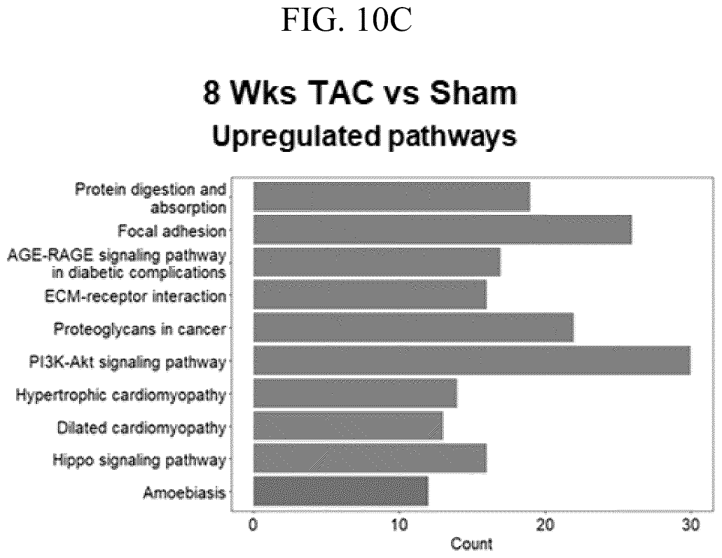

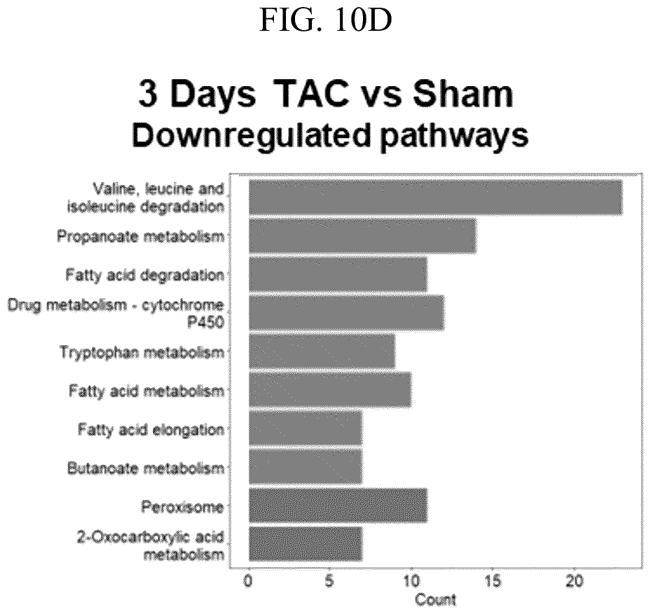

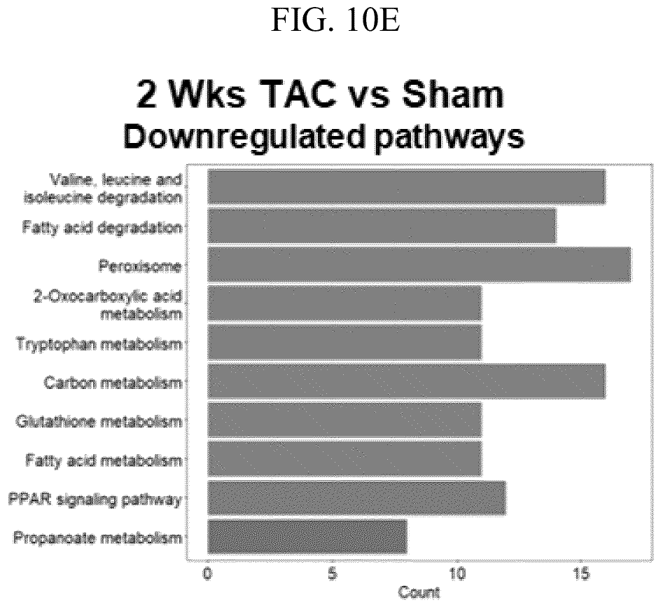

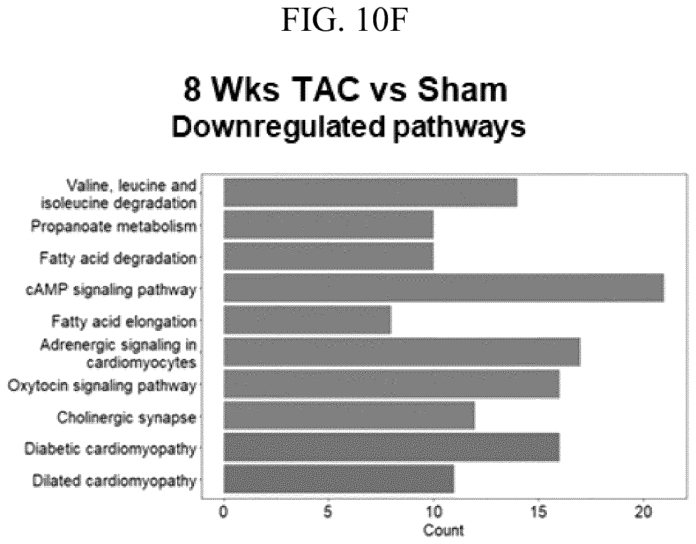

FGF21 elevation is accompanied by myocardial fibrosis, metabolic derangement, and accumulation of toxic lipid species—To assess the gene expression changes that FGF21 stimulates in the heart over the course of cardiac hypertrophy progression, transcriptomic analysis was conducted in mouse hearts at 3 days, 2 weeks, and 8 weeks post-TAC. A total of 1831 genes, 1717 genes and 1186 genes were differentially expressed at 3 days, 2 weeks, and 8 weeks post-TAC, respectively. Volcano plot and hierarchical clustering analyses identified distinct transcriptomic profiles among the 3 experimental groups. Furthermore, genes that are involved in fibrosis and hypertrophy were among the top-50 differentially expressed genes in the early and late phases of hypertrophy progression. Specifically, the expression of Col3a1, Col8a1 and Fbn1 was upregulated in 3 days, Col15a1, Nppa and Dpys13 expression was upregulated at 2 weeks post-TAC, and Acta1, Ankrd1, and Flnc were increased at 8 weeks post-TAC. KEGG pathway analysis revealed consistent upregulation of cardiac Extracellular Matrix (ECM)-interaction pathways across all time points (FIG. 2A, FIGS. 10A-10C). Notably, 2 weeks and 8 weeks post-TAC, there was a pronounced increase in gene expression of components of pathways associated with PI3-AKT, dilated cardiomyopathy, and hypertrophic cardiomyopathy signaling (FIGS. 10B and 10C). Contrary to the hypertrophic signaling enrichment, expression of genes that are related to catabolism of branched-chain amino acids (BCAA), such as valine, leucine, and isoleucine or catabolism of fatty acids was consistently downregulated across all time points (FIGS. 10D-10F).

### Example 3

To corroborate our transcriptional profiling with the metabolic profile of the heart, we performed metabolomic and lipidomic analyses in the same sets of samples at 3 days, 2 weeks, and 8 weeks post-TAC. Metabolomic analysis showed that 3 days post-TAC, myocardial glucose content is significantly lower compared to control mice with sham surgery and so is the glucose derivative, pyruvate, as well as citrate and NAD+ (FIG. 2B, Table 6). On the other hand, p-hydroxybutyrate (BHB, ketone body) seems to accumulate 3 days post-TAC (FIG. 2B, Table 6). At 2 weeks post-TAC, glucose levels remain lower compared to sham (FIG. 2B, Table 7). Conversely, succinate, as well as derivatives of glutamine and glutamate metabolism, such as asparagine and serine, are increased (FIG. 2B, Table 7). Notably, 8 weeks post-TAC when pathological hypertrophy has fully developed, cardiac BCAAs (valine, leucine, isoleucine) accumulate and so are serine, asparagine and glutathione (another derivative of glutamate) (FIG. 2B, Table 8). Accumulation of these nutrients is accompanied by lower levels of both NAD+ and ATP, implying lower electron transport towards oxidative phosphorylation and ATP synthesis.

Lipidomic analysis showed that although total lipid species do not change significantly, certain lipid species are altered at various time points (FIG. 2B, Table 9-11). Triglyceride (TG) species that change 3 days post-TAC are mostly increased, while diacylglycerol (DAG) species are decreased implying less TG hydrolysis (FIG. 2B, Table 9). At 2 weeks post-TAC, 14 TG and 2 DAG species show lower abundance, which coincides with increase of 24 phosphatidyl-choline (PC), 27 phosphatidyl-ethanolamine (PE), and 1 phosphatidyl-serine (PS) species (FIG. 2B, Table 10). These changes in phospholipid species at 2 weeks post-TAC may reflect the increased need for membrane synthesis in hearts that hypertrophy has started (FIG. 1D). At 8 weeks post-TAC, there is still higher abundance of phospholipid species, including 13 PC, 12 PE and 2 PS (FIG. 2B, Table 11). The cardiac lipidome changes at the final stage of cardiac hypertrophy (8 weeks post-TAC) also include accumulation of acetyl-carnitine (precursor of carnitine; carnitine facilitates transportation of fatty acyl-CoAs in mitochondria for β-oxidation), lower abundance of 3 fatty acyl-carnitine species and 3 species of cardiolipin, which is a phospholipid of the inner mitochondrial membrane (FIG. 2B, Table 11). Overall, combined transcriptomic, metabolomic and lipidomic analysis suggests that during progression of cardiac hypertrophy, the reprogramming of the cardiac metabolic pathways includes accumulation of β-hydroxybutyrate and lower glucose content at the early stage followed by accumulation of BCAAs, phospholipid species and byproducts of the glutamine—glutamate—α-ketoglutarate pathway, as well as by lower acyl-carnitines, NAD+ and ATP content at the late stage.

### Example 4

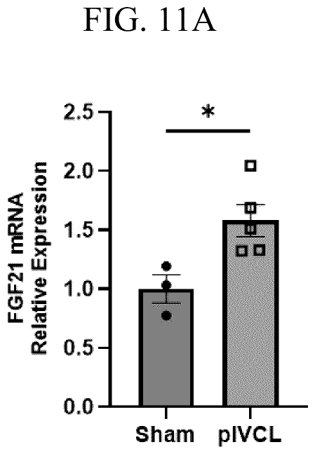

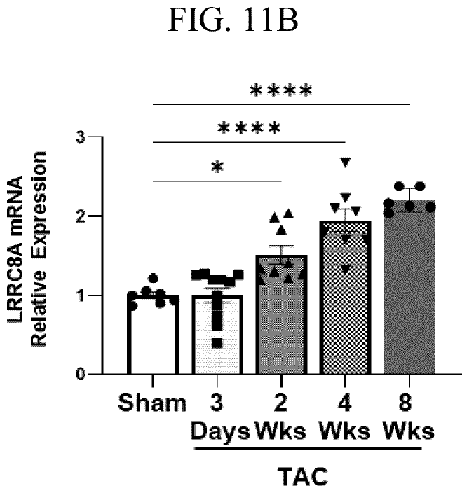

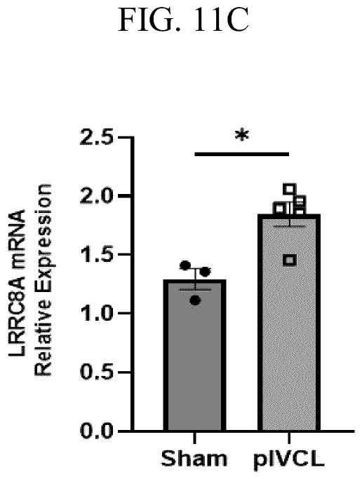

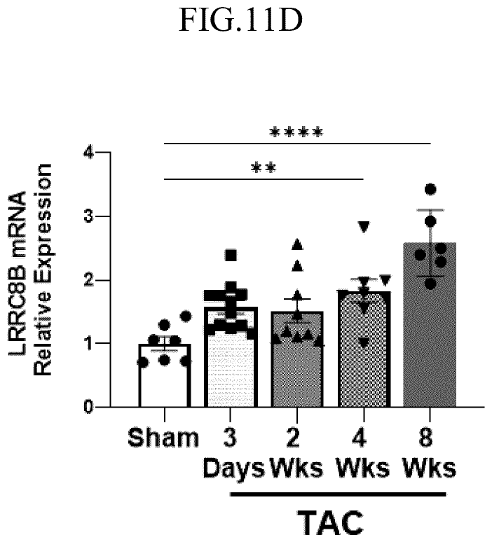

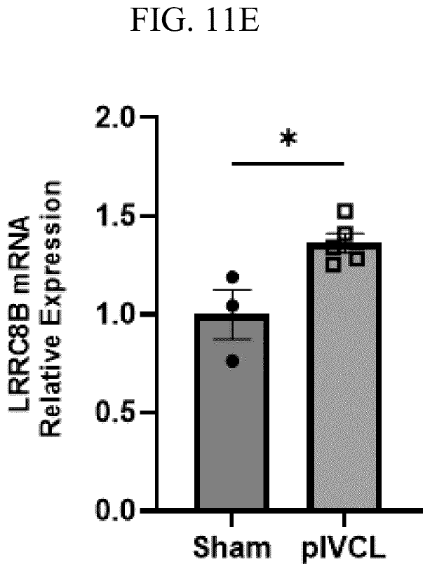

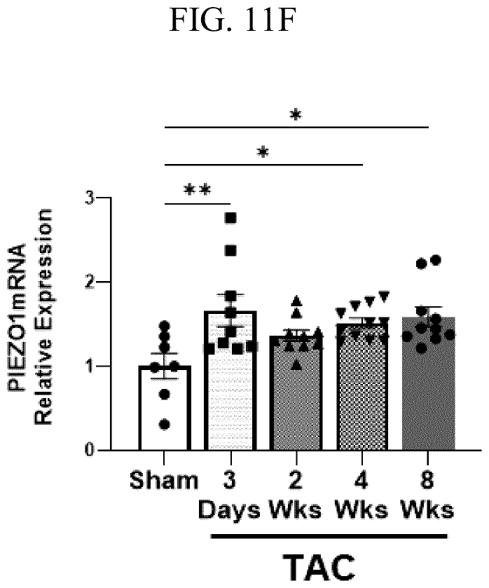

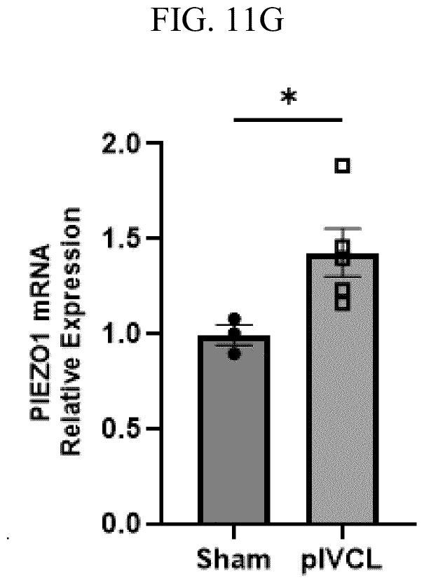

Hepatic FGF21 induction is associated with liver congestion. To investigate whether hemodynamic congestion in the liver, which occurs with TAC, may be the cause for the induction of hepatic FGF21 expression, we performed partial inferior vena cava ligation (pIVCL) as an alternative way to induce hepatic congestion. Mice with pIVCL showed significant upregulation of hepatic FGF21 mRNA levels compared to control sham mice (FIG. 11A). Further analyses in mice with either TAC or pIVCL showed significant increase of the hepatic gene expression of the mechano-sensors Leucine-rich repeat-containing protein (LRRC)8A, LRRC8B and Piezo-type mechanosensitive ion channel component (PIEZO)1 (FIGS. 11B-11G). Thus, hemodynamic congestion in the liver increases hepatic expression of FGF21, which is associated with elevated expression of mechano-sensor proteins.

### Example 5

Inhibition of hepatic FGF21 lowers circulating FGF21 levels and alleviates cardiac hypertrophy with pressure overload. Next, we investigated whether FGF21 upregulation is a cause or a consequence of heart failure in mice with TAC. Because hepatocyte FGF21 upregulation precedes the development of cardiac hypertrophy and LV dysfunction, we studied the effect of LV pressure overload in mice lacking hepatocyte FGF21 that we generated (HEP-FGF21−/−) by crossing Alb-Cre+/− mice with Fgf21f1/f1 mice. First, we confirmed that deletion of Fgf21 in the liver lowered hepatic FGF21 expression (FIG. 12A) without affecting FGF21 expression in the heart and WAT (FIGS. 12B and 12C). As the control group of mice included a combination of Fgf21f1/f1 and Alb-Cre+/− mice, we tested whether there are strain-specific differences that would preclude us from combining them in the control experimental groups. Our analyses showed that the Fgf21f1/f1, the Alb-Cre+/− as well as control αMHC-Cre+/− mice exhibit similar responses to TAC surgery as shown by HW/TL (FIG. 12D), systolic dysfunction (FIG. 12E), cardiac expression of 3-MHC (FIG. 12F) and COL3A1 (FIG. 12G). Thus, our control groups for this study include a combination of floxed and Cre-expressing mice.

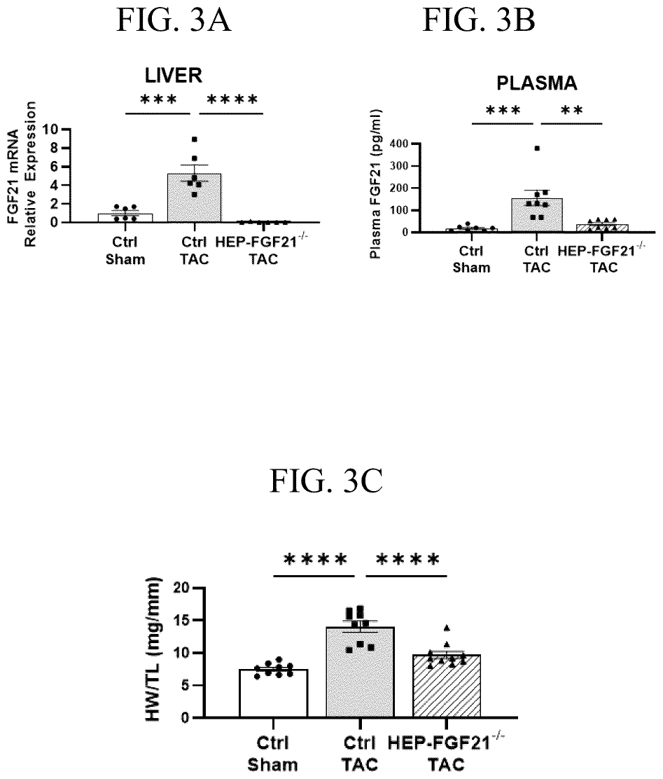

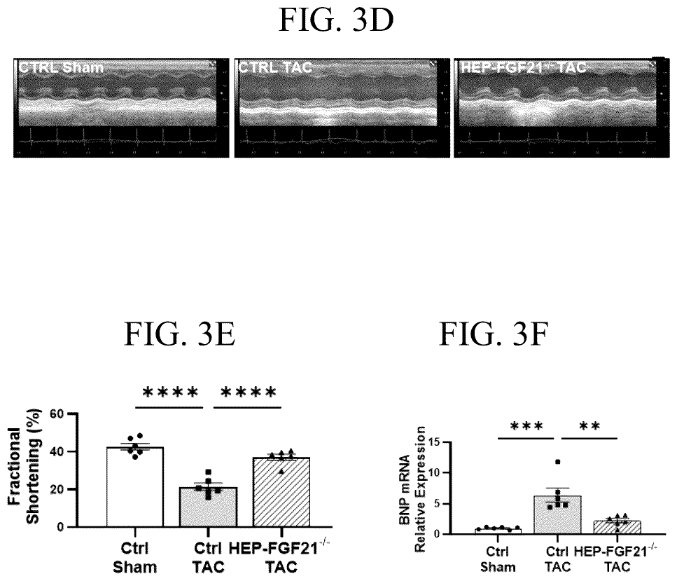

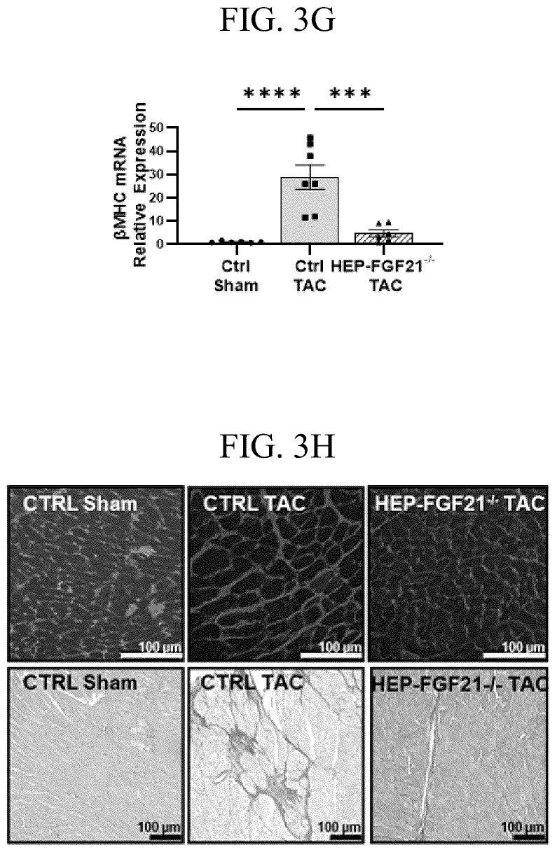

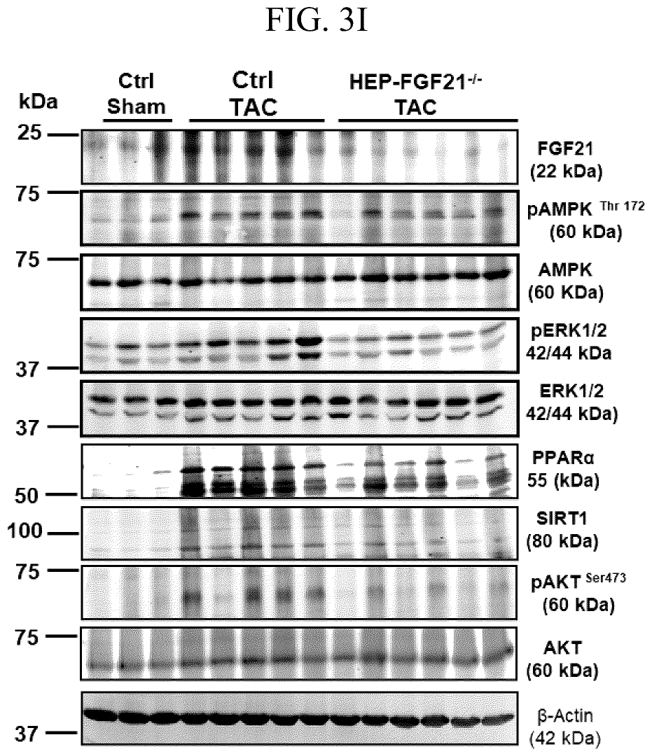

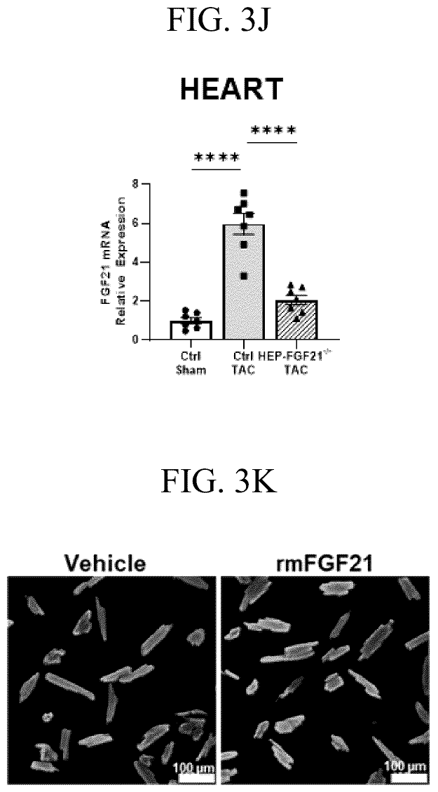

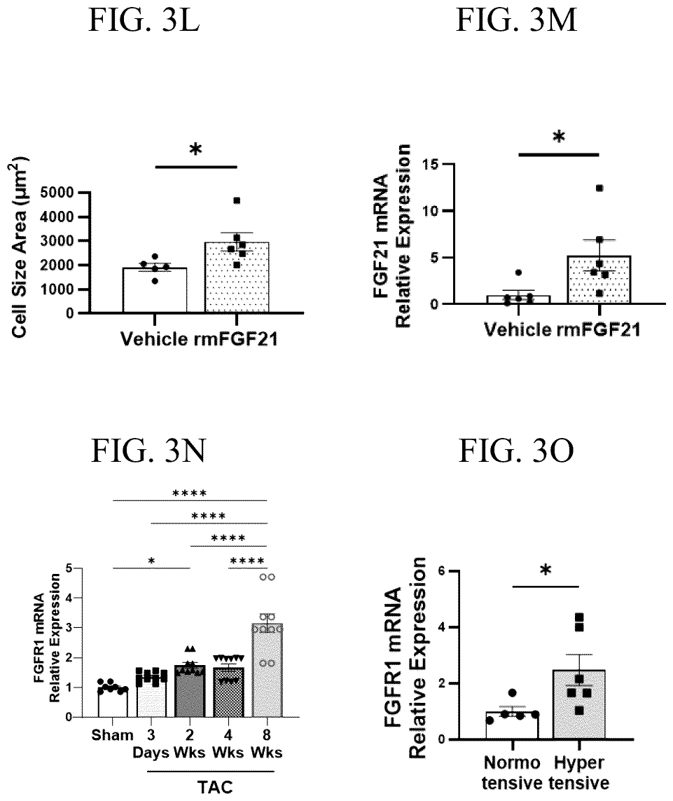

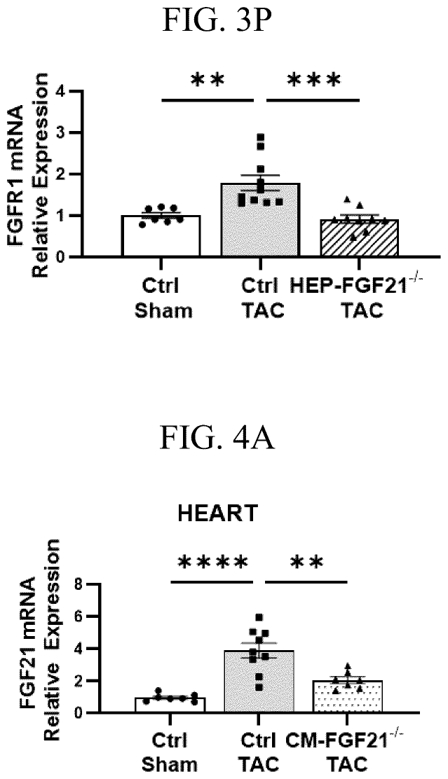

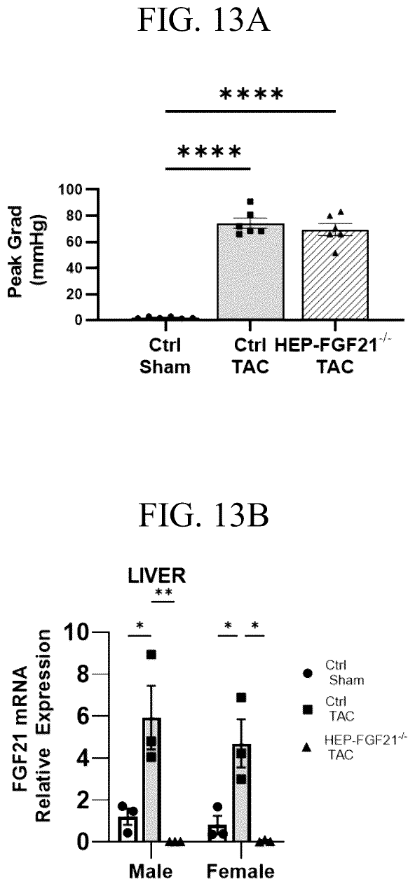

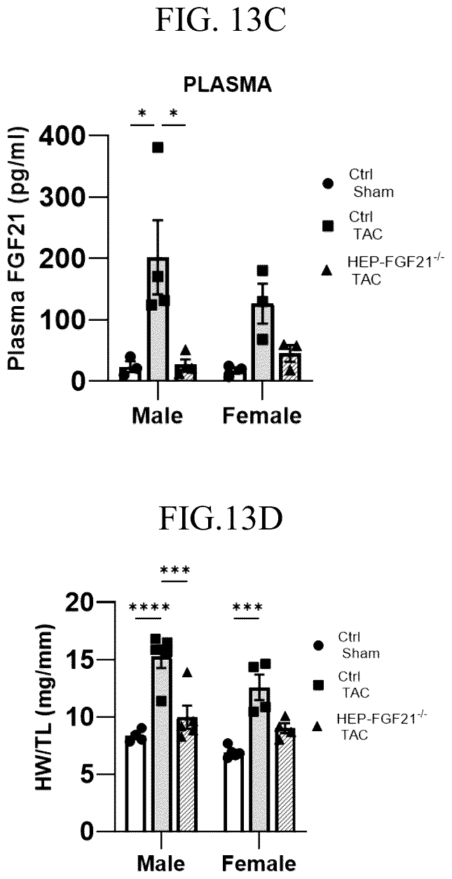

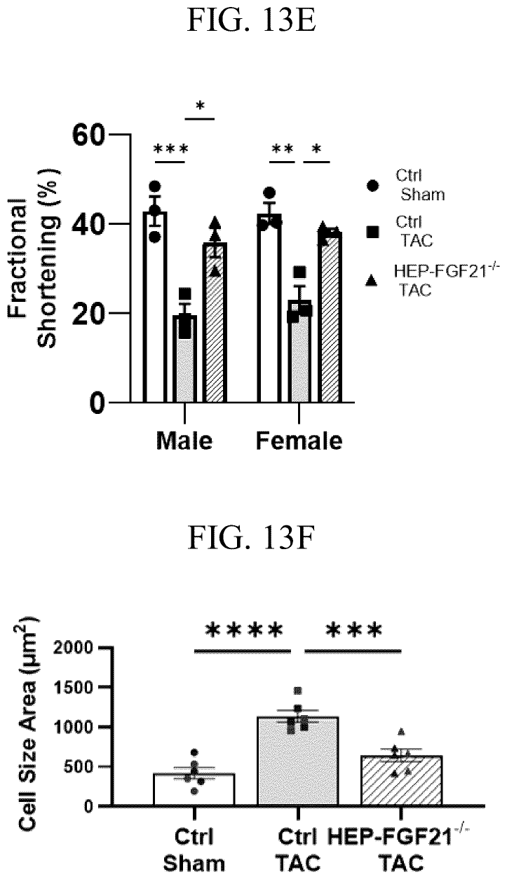

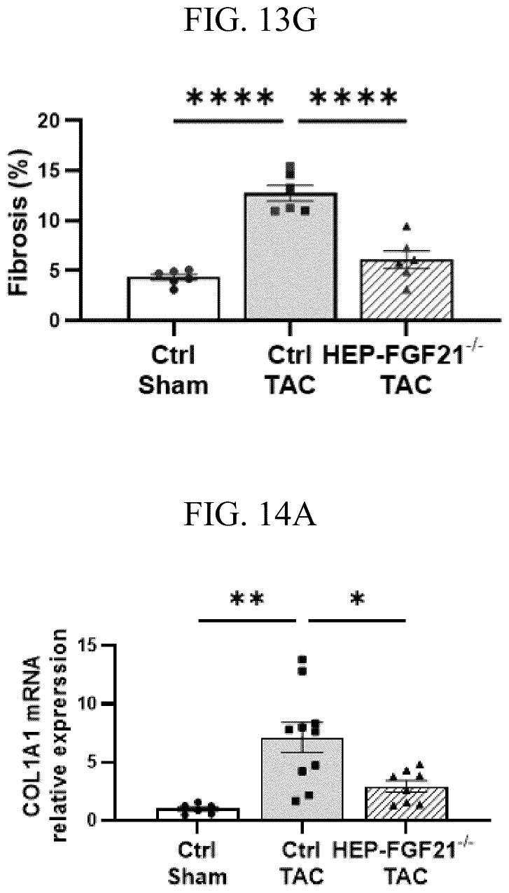

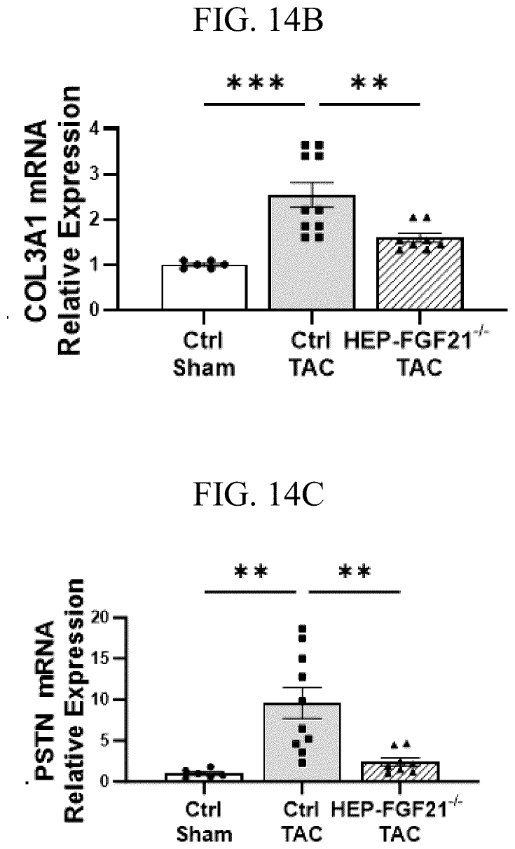

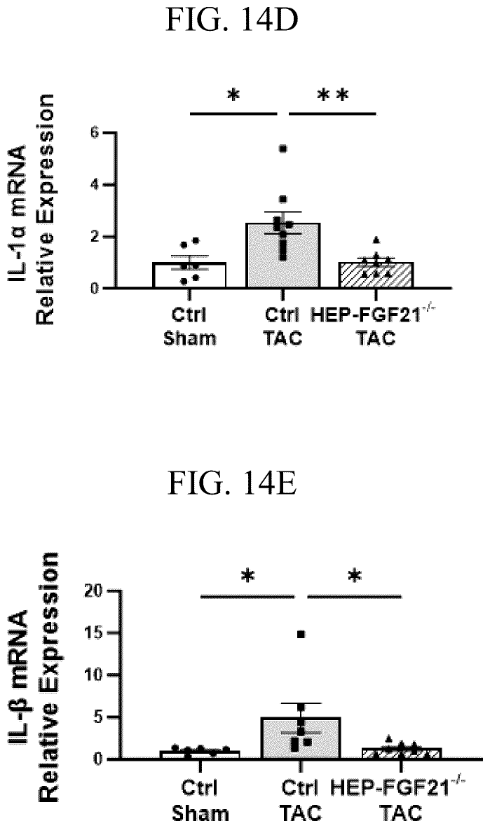

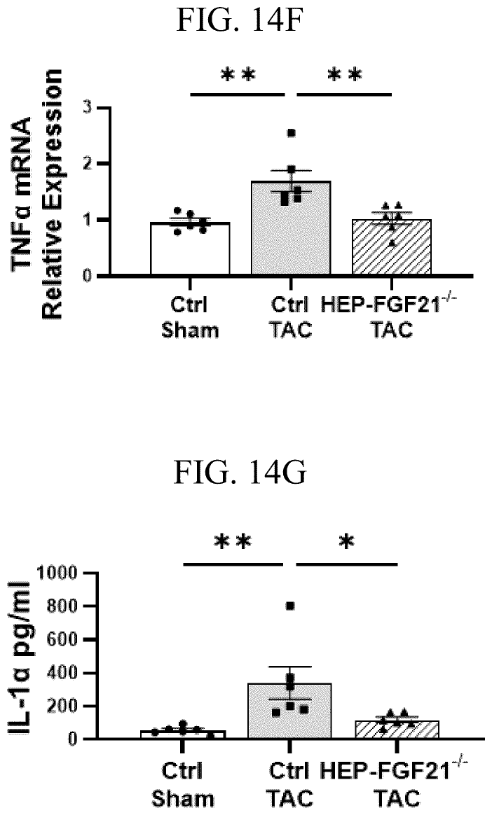

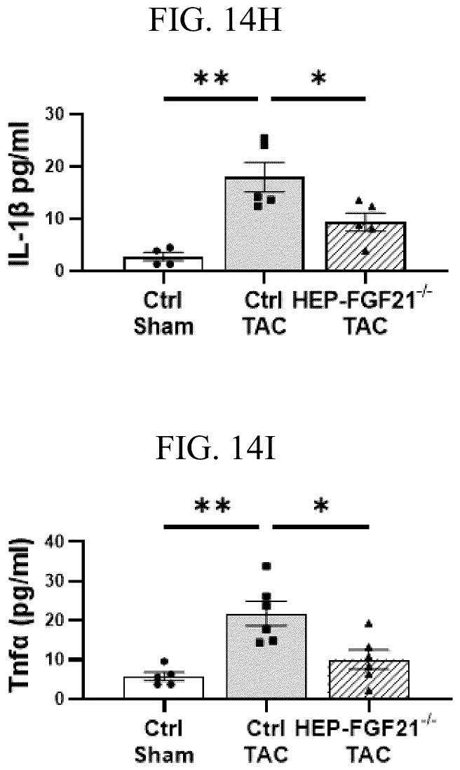

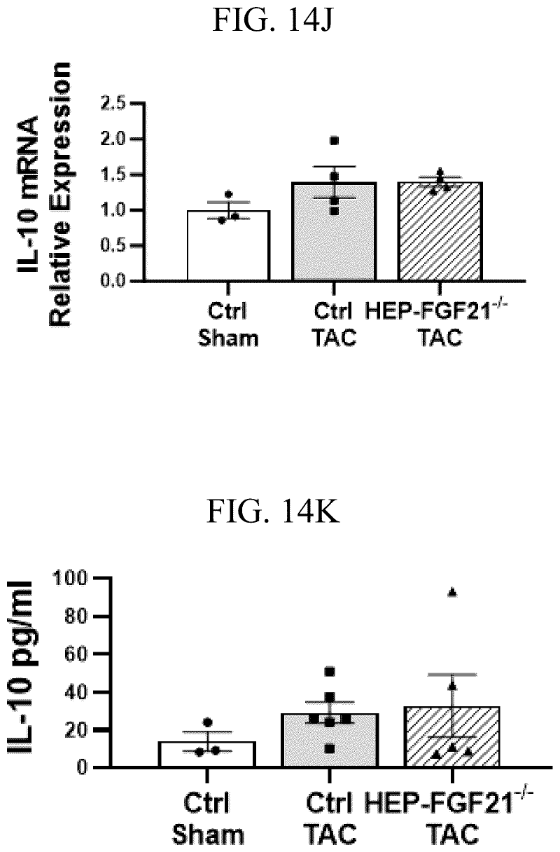

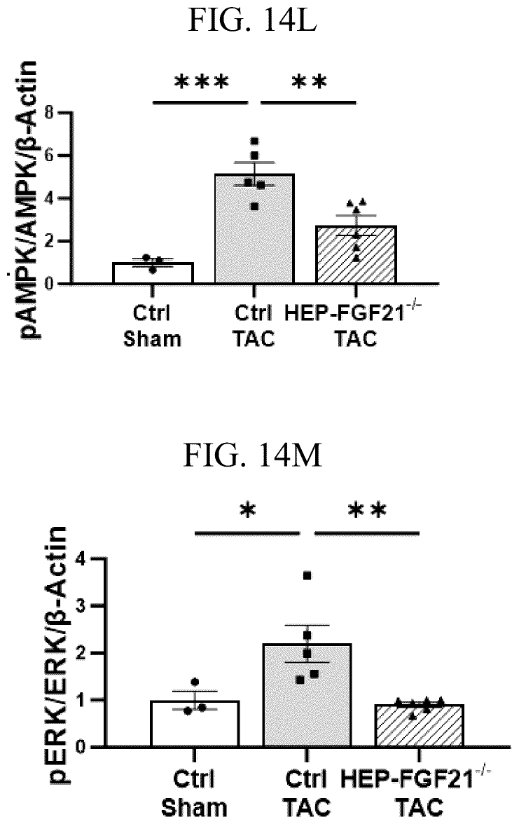

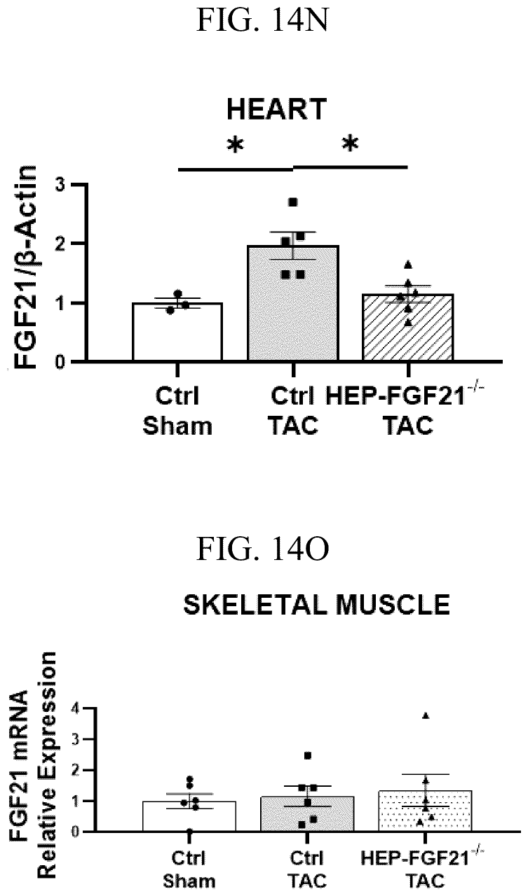

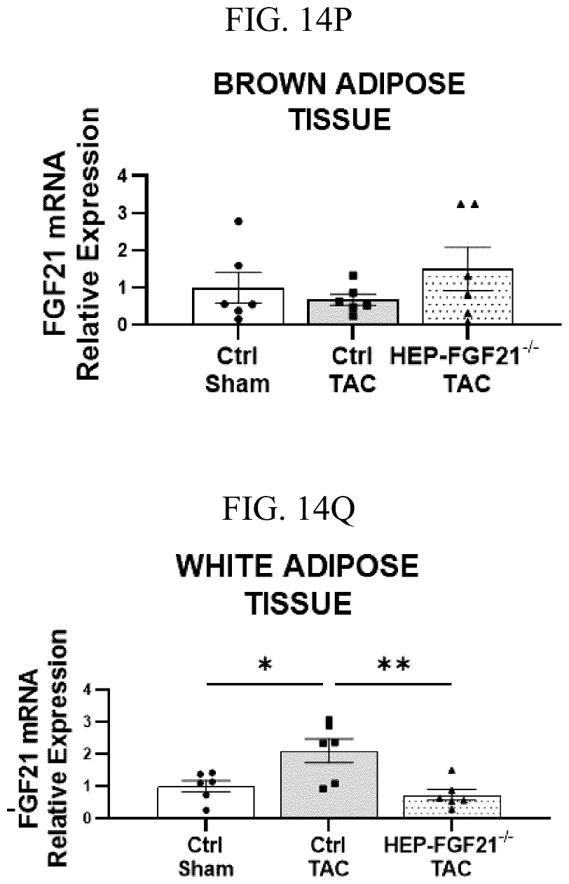

Then, we applied TAC in HEP-FGF21−/− and control mice for 8 weeks and confirmed equivalent increase in pressure gradient (FIG. 13A). Hepatocyte-specific deletion of FGF21 prevented elevation of FGF21 expression in the liver (FIG. 3A) and maintained plasma FGF21 levels within the normal range (FIG. 3B) when TAC was applied. Strikingly, HEP-FGF21−/− mice showed less profound cardiac hypertrophy (FIG. 3C) and cardiac function was protected (FIGS. 3D and 3E and Table 12, Table 13). Notably, no sex-specific differences were observed in the HEP-FGF21−/− mice following TAC, either in circulating FGF21, tissue expression of FGF21 or in cardiac function and hypertrophic response as shown by hepatic FGF21 expression (FIG. 13B), circulating FGF21 levels (FIG. 13C), HW/TL (FIG. 13D), and cardiac function (FIG. 13E). Alleviation of cardiac hypertrophy and improvement of heart function in HEP-FGF21−/− mice were accompanied by lower cardiac expression of heart failure markers, BNP (FIG. 3F) and 3-MHC (FIG. 3G), attenuation of cardiomyocyte enlargement (FIG. 3H, FIG. 13F), less myocardial fibrosis (FIG. 3H, FIG. 13G), reduced fibrosis-related gene expression (COL1A1, COL3A1, PSTN) (FIG. 14A-C), lower cardiac expression (FIGS. 14D-14F) and circulating levels (FIGS. 14G-14I) of Interleukin (IL)-1α, IL-1β, and TNFα inflammatory cytokines, while IL-10 was not altered (FIGS. 14J and 14K) compared to control mice with TAC. Accordingly, prohypertrophic signaling was attenuated in hearts of HEP-FGF21−/− mice with TAC, as shown by lower pAMPK/tAMPK (FIG. 3I, FIG. 14L) and pERK/tERK ratio (FIG. 3I, FIG. 14M) compared to control mice with TAC.

Surprisingly, the hearts of HEP-FGF21−/− mice showed almost complete prevention of FGF21 mRNA (FIG. 3J) and protein (FIG. 3I, FIG. 14N) induction with TAC. On the other hand, no significant changes were observed in FGF21 mRNA levels in the skeletal muscle (FIG. 14O) and BAT (FIG. 14P). However, induction of FGF21 mRNA in the WAT that is observed in control mice with TAC (FIG. 1K), was absent in HEP-FGF21−/− mice (FIG. 14Q).

### Example 6

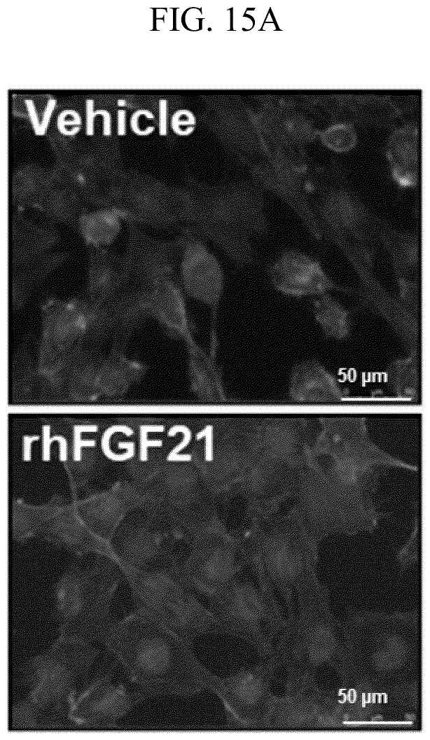

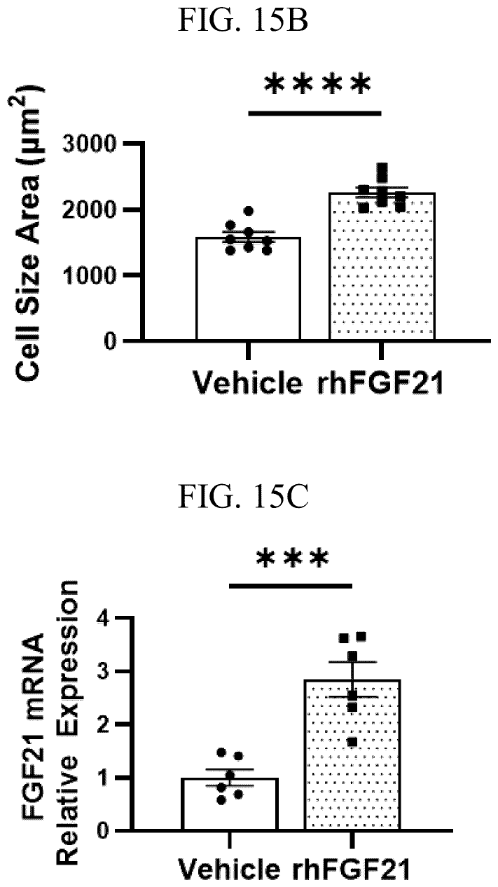

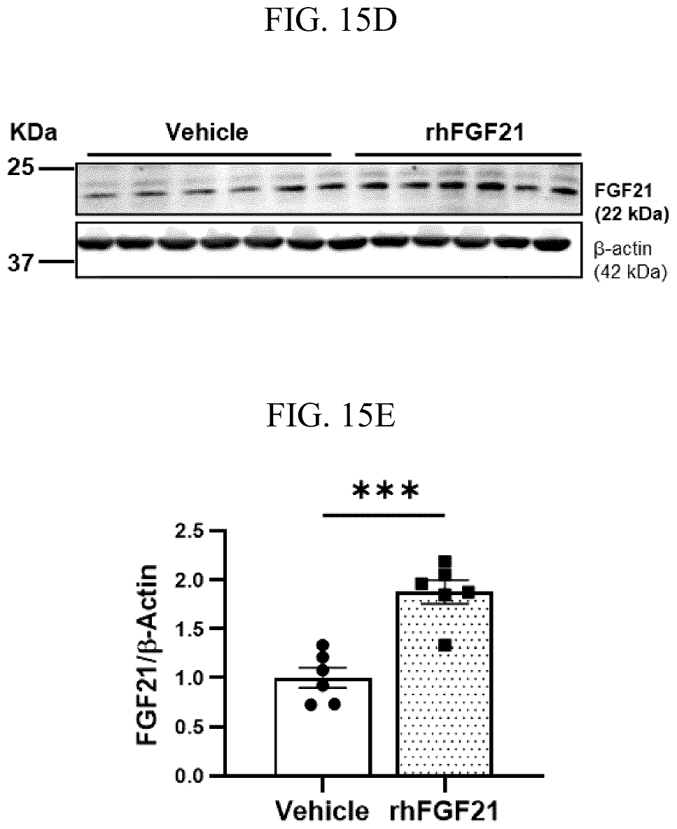

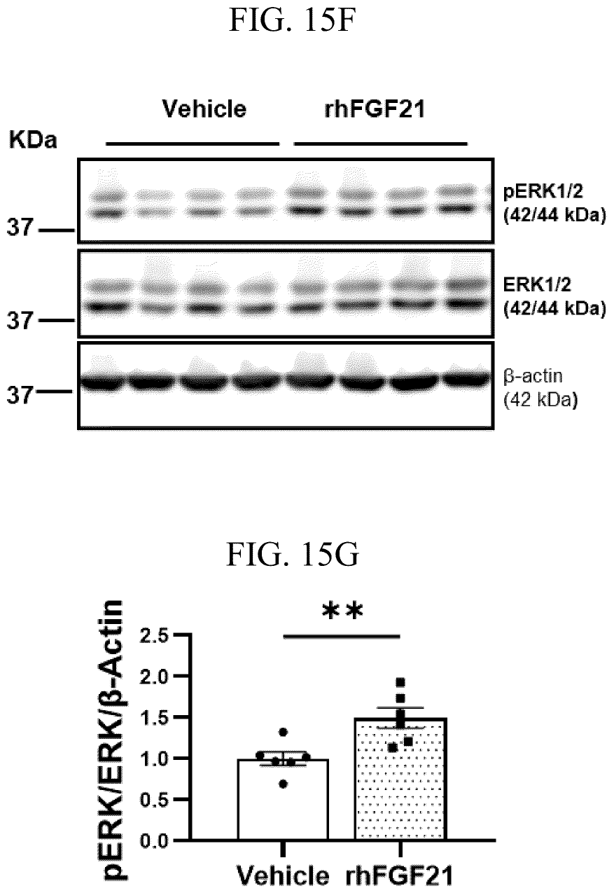

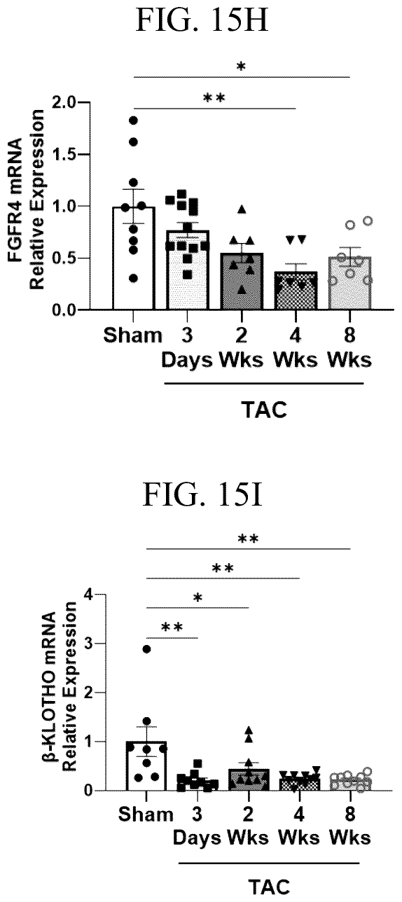

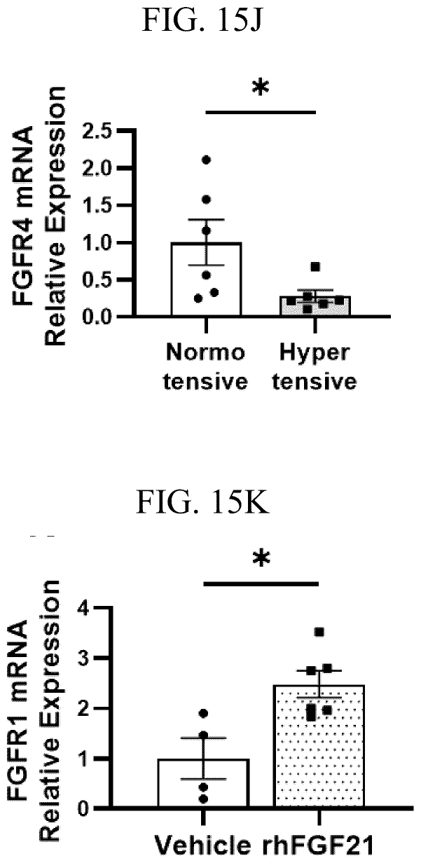

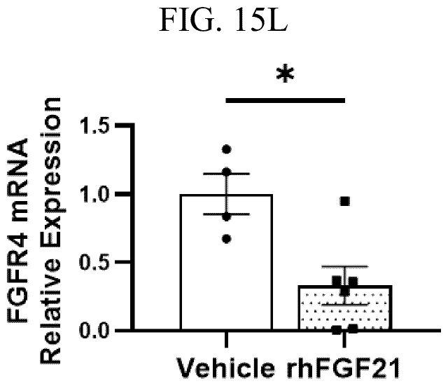

Liver-derived FGF21 controls cardiomyocyte FGF21 expression. The similar pattern of cardiac and hepatic FGF21 expression prompted us to determine whether this was due to the improvement of cardiac function or circulating (plasma) FGF21 can directly affect FGF21 expression (mRNA) in cardiomyocytes. To this end, we treated primary cardiomyocytes that we isolated from adult mice and a human cardiomyocyte cell line (AC16) with recombinant mouse (rm) and recombinant human (rh) FGF21, respectively, in vitro. In both cases FGF21 promoted cardiomyocyte enlargement (FIGS. 3K and 3L, FIGS. 15A and 15B). Moreover, FGF21 treatment stimulated endogenous FGF21 expression in both primary cardiomyocytes (FIG. 3M) and AC16 cells (FIGS. 15C-15E). The pro-hypertrophic effect of FGF21 was accompanied by increased pERK/tERK ratio, a known downstream signaling pathway of FGF21 (FIGS. 15F and 15G). Thus, inhibition of hepatic FGF21 alleviates pressure overload-induced hypertrophy, which is associated with suppression of cardiac FGF21 expression that seems to be driven by the lower circulating FGF21 levels.

### Example 7

To explore further how cardiomyocytes sense exogenous FGF21 signaling, we assessed the expression of the main cardiomyocyte FGF21 receptors (FGFR). This analysis showed that cardiac FGFR1 expression was significantly upregulated 2 weeks post-TAC when cardiac FGF21 is also increased (FIG. 3N) and it was further elevated 4 and 8 weeks post-TAC (FIG. 3N). Conversely, the expression of another important cardiac FGF21 receptor, FGFR4, was gradually suppressed as LVH progressed (FIG. 15H) and so was 0-klotho mRNA levels (FIG. 15I). Accordingly, FGFR1 expression was elevated, while FGFR4 expression was reduced in myocardial tissue from individuals with hypertension (FIG. 3O, FIG. 15J) and in AC16 cells treated with rhFGF21 (FIGS. 15K and 15L). Notably, cardiac FGFR1 expression was not upregulated in HEP-FGF21−/− mice with TAC (FIG. 3P). Thus, FGF21-mediated LVH is associated with increased FGFR1 expression.

### Example 8

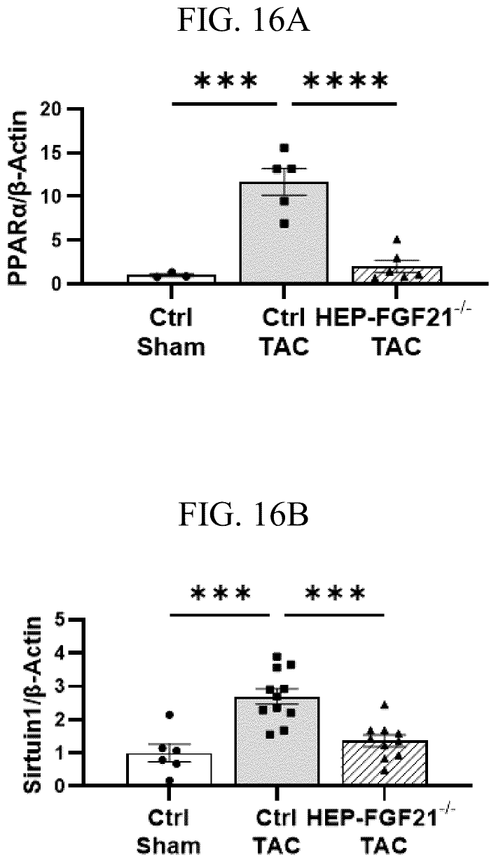

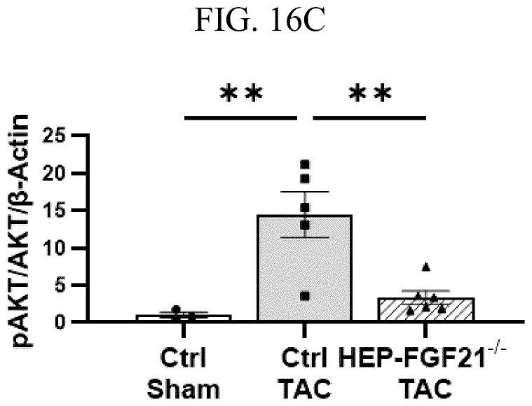

As FGF21 expression is induced by activation of PPARα, Sirtuin (SIRT), and Akt signaling, we measured the protein levels of these regulators in the hearts of control mice with TAC, as well as in HEP-FGF21−/− mice. These analyses showed that cardiac PPARα (FIG. 3I, FIG. 16A), SIRT1 (FIG. 3I, FIG. 16B) and pAkt/tAkt (FIG. 3I, FIG. 16C), expression was increased in control mice with TAC. The increase of these FGF21 regulators was prevented (FIG. 3I, FIGS. 16A-16C) in HEP-FGF21−/− mice that had lower plasma FGF21 levels and were protected from TAC-induced LVH. Thus, systemic FGF21 upregulation that follows pressure overload activated cardiac PPARα, SIRT1 and pAkt/tAkt, signaling, which was negated upon normalization of plasma FGF21 levels.

### Example 9

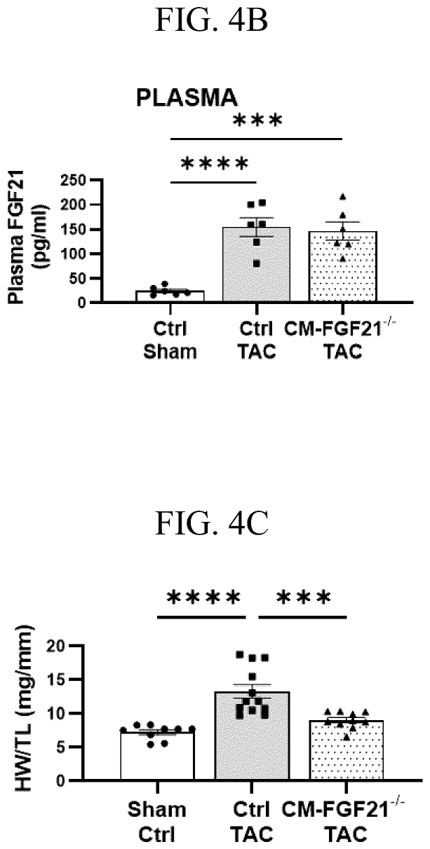

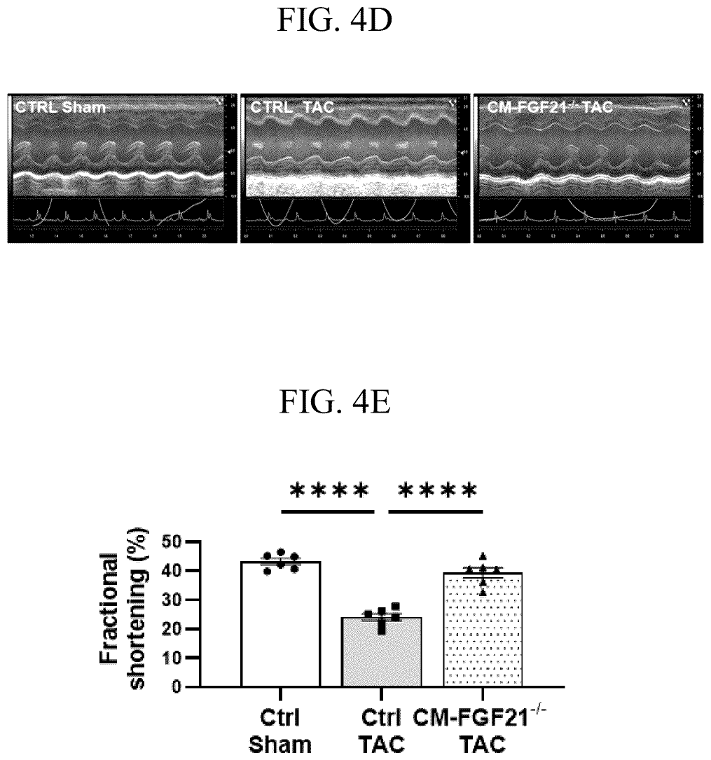

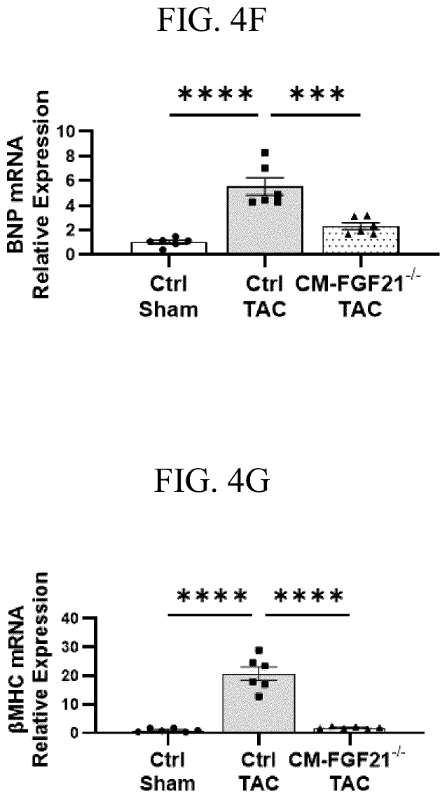

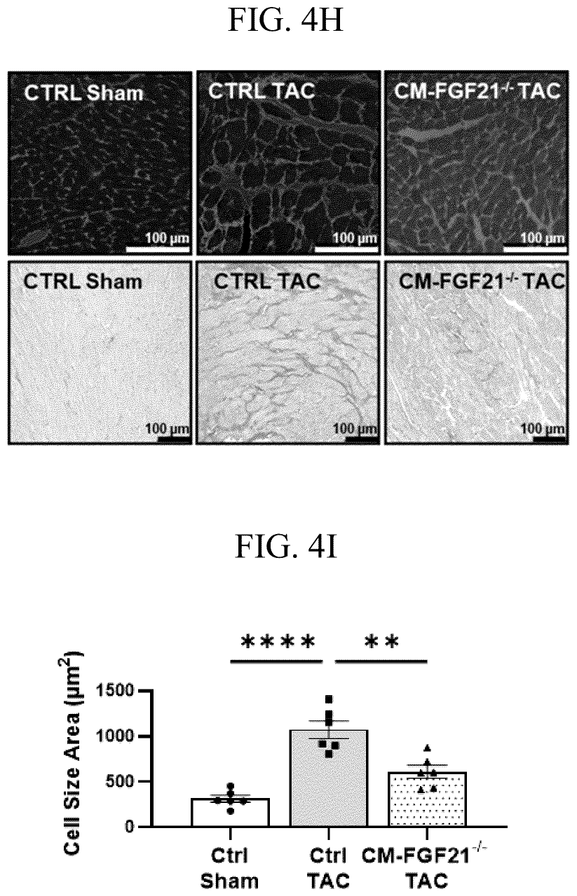

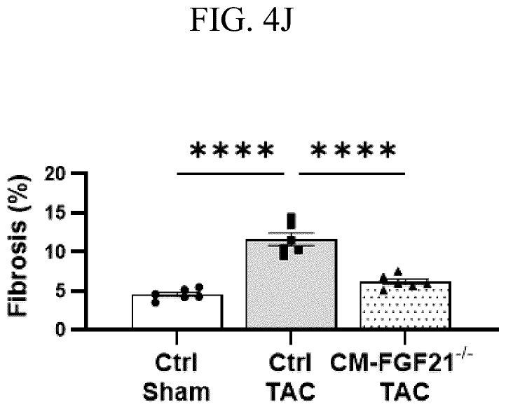

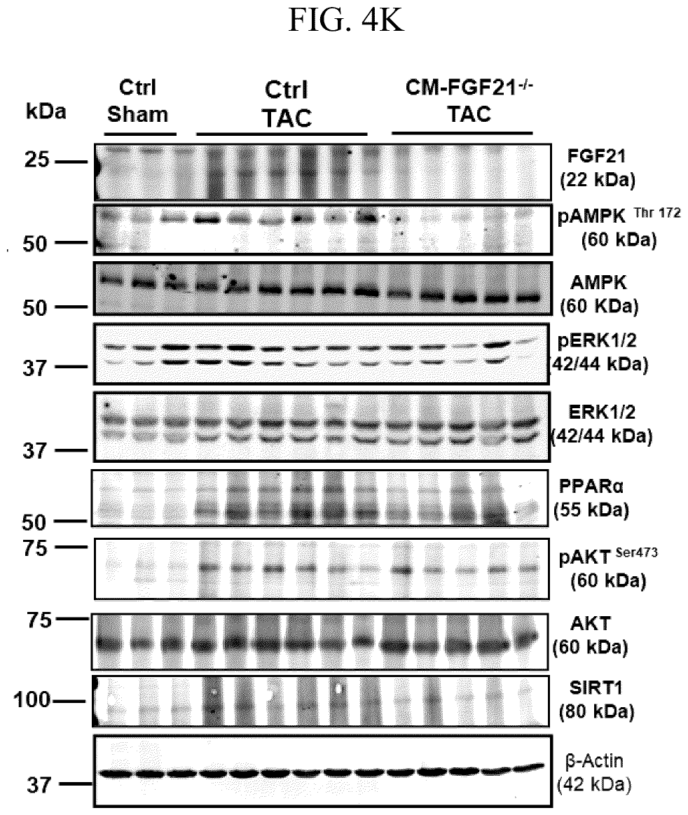

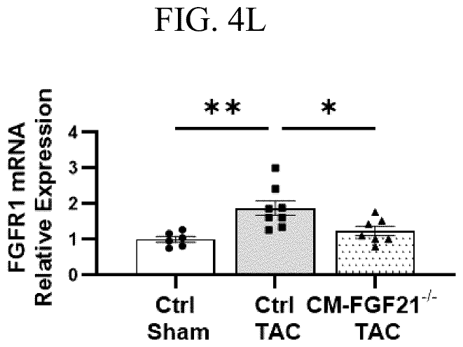

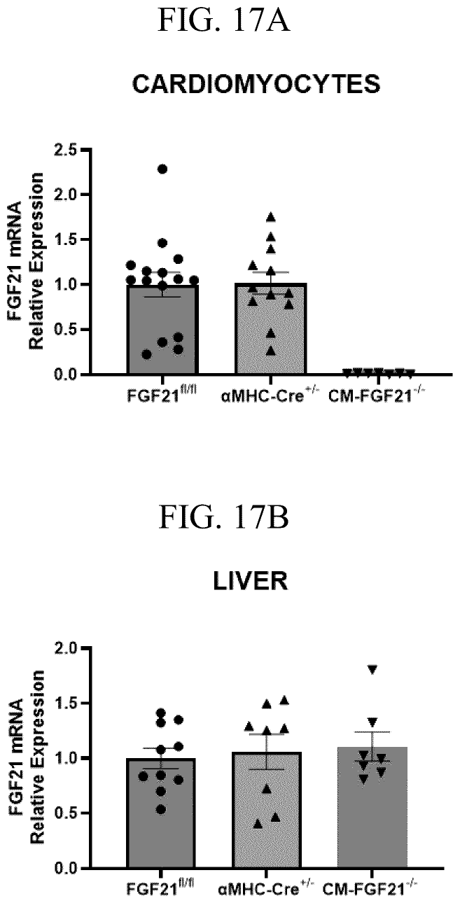

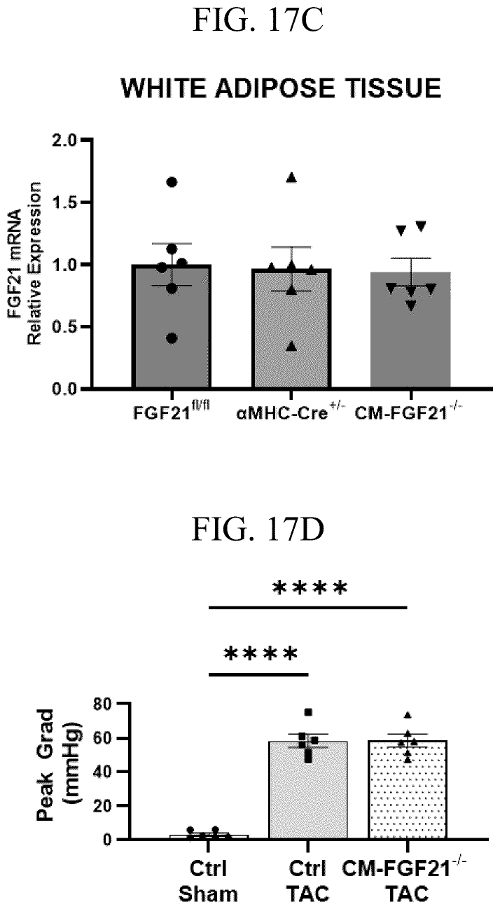

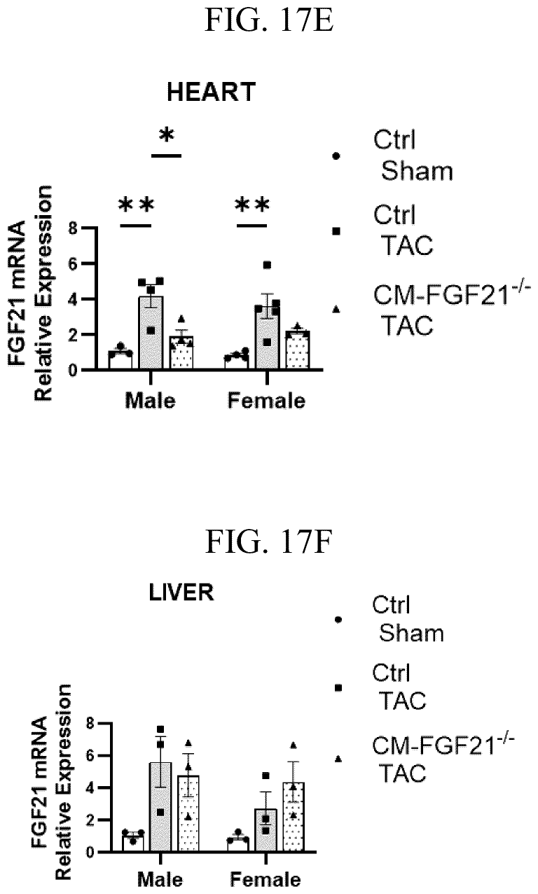

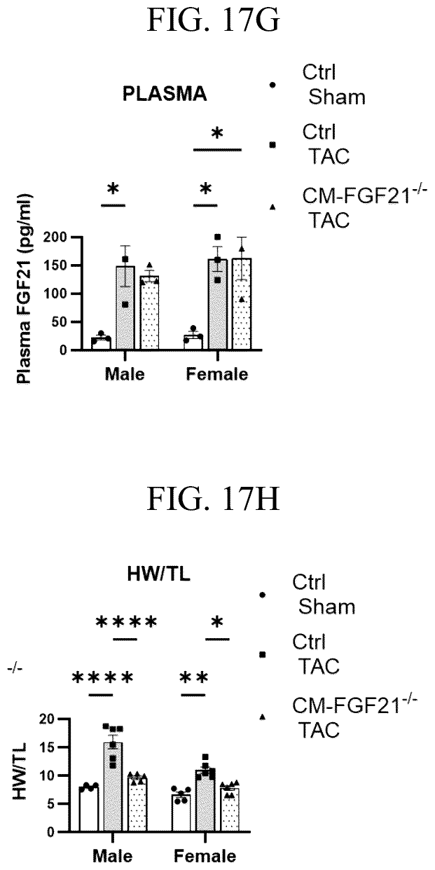

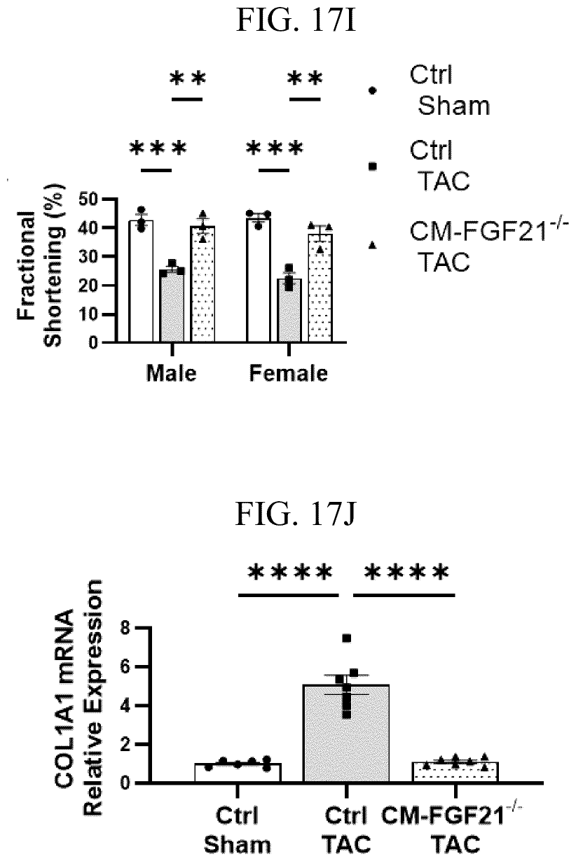

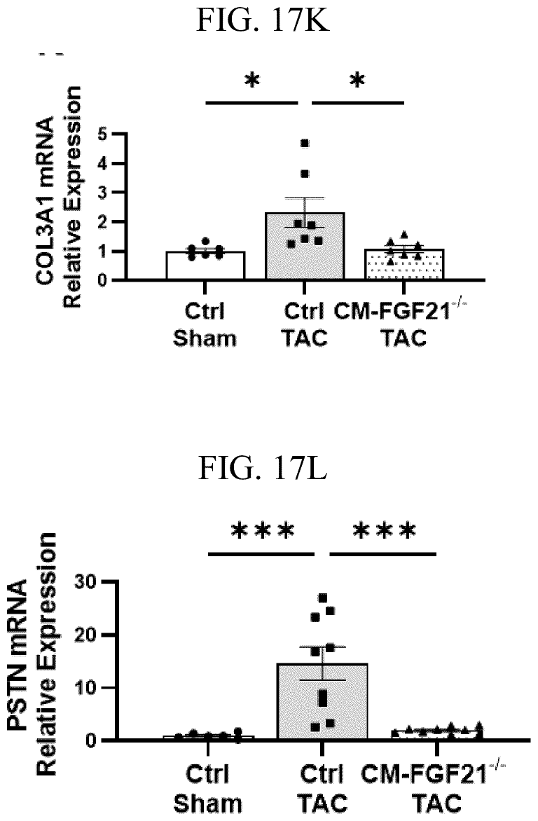

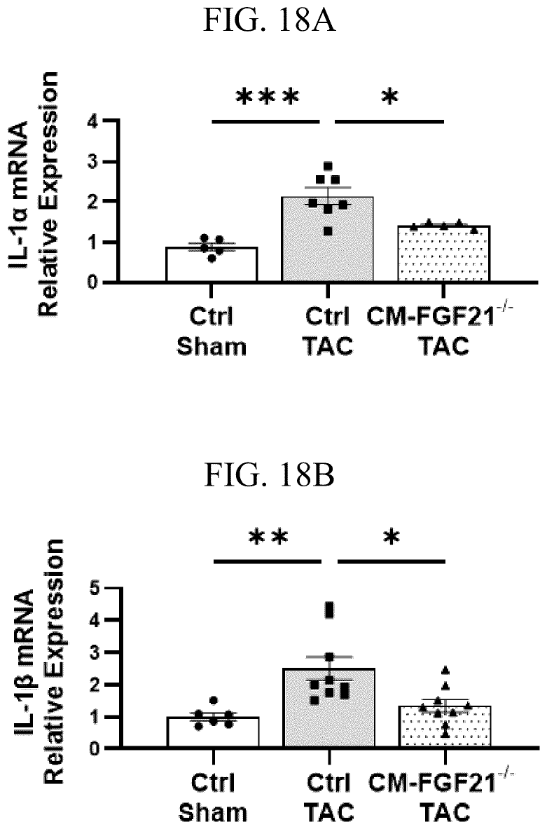

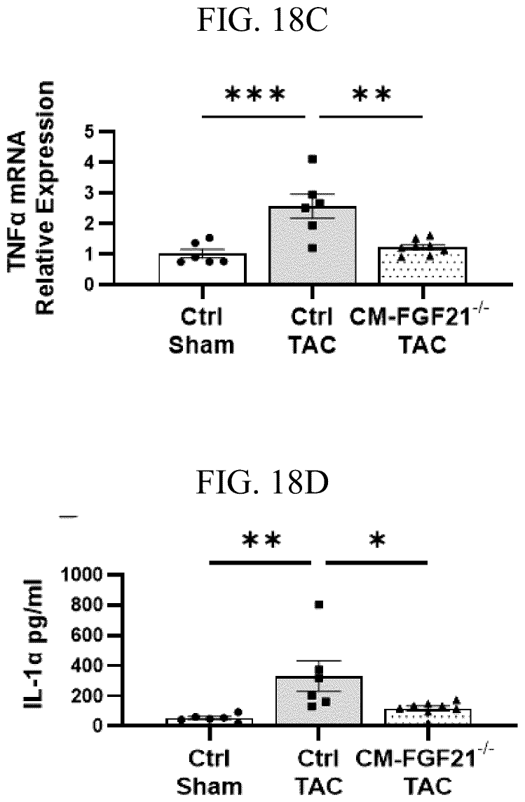

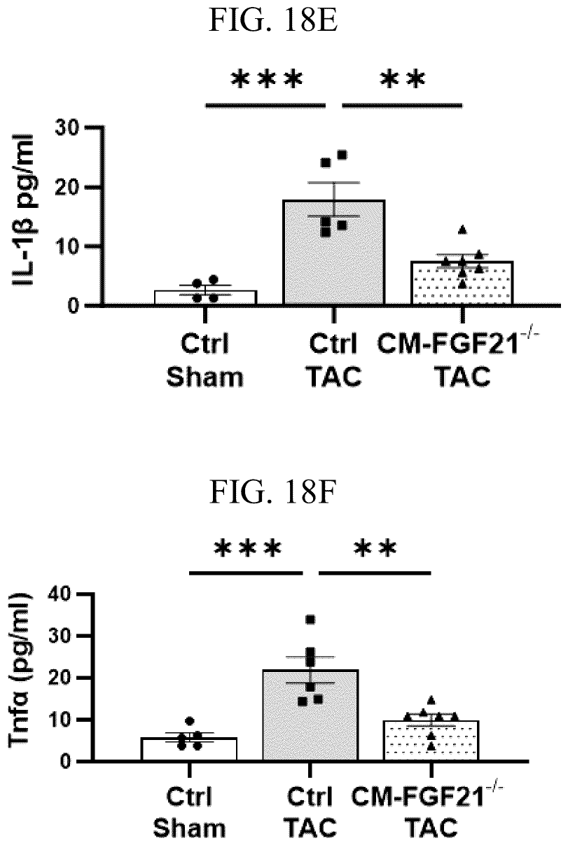

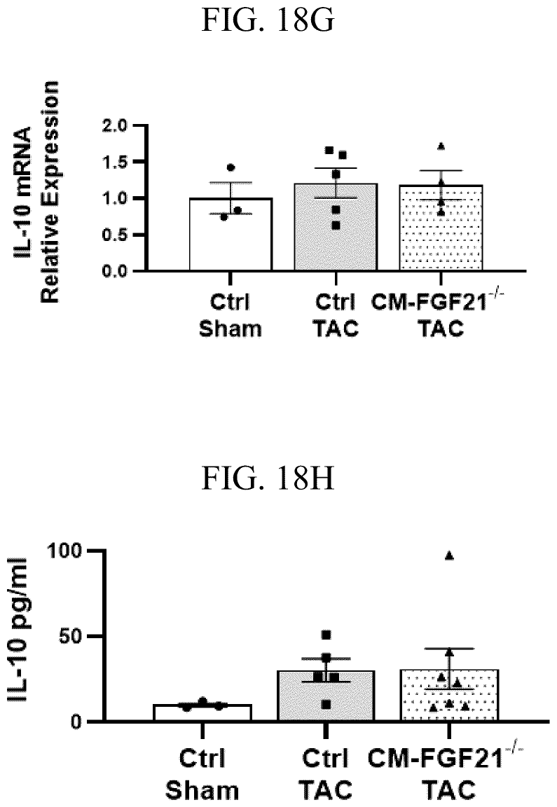

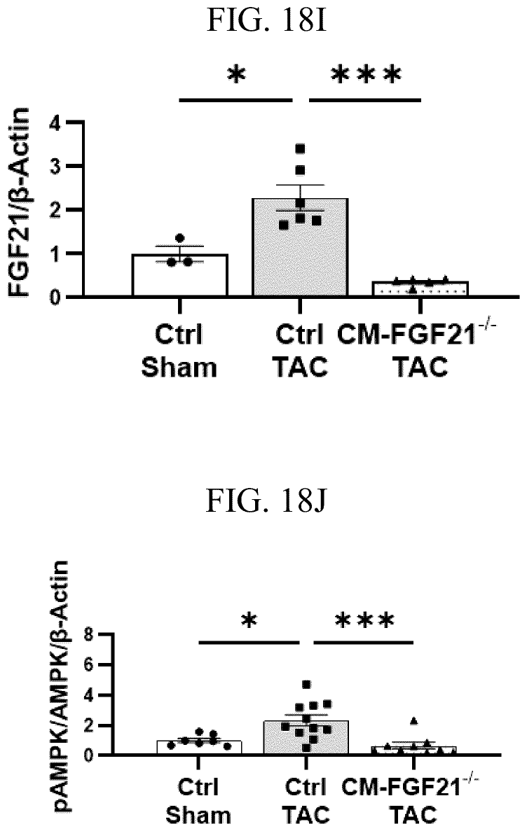

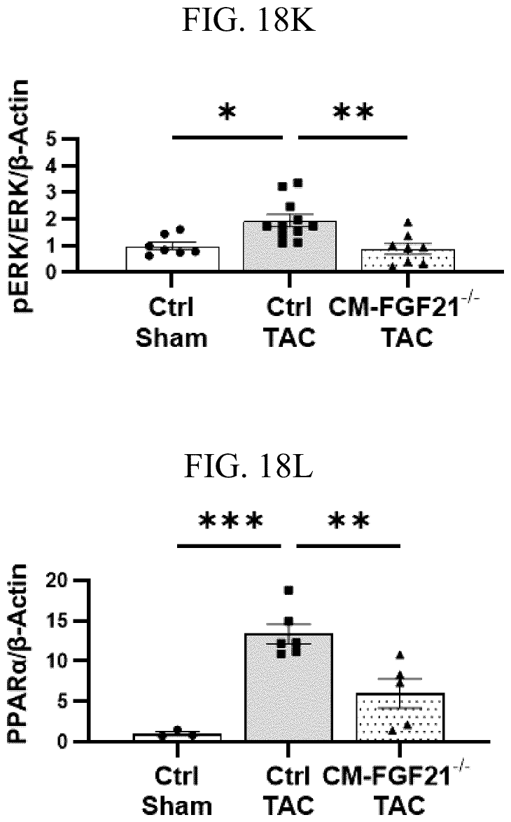

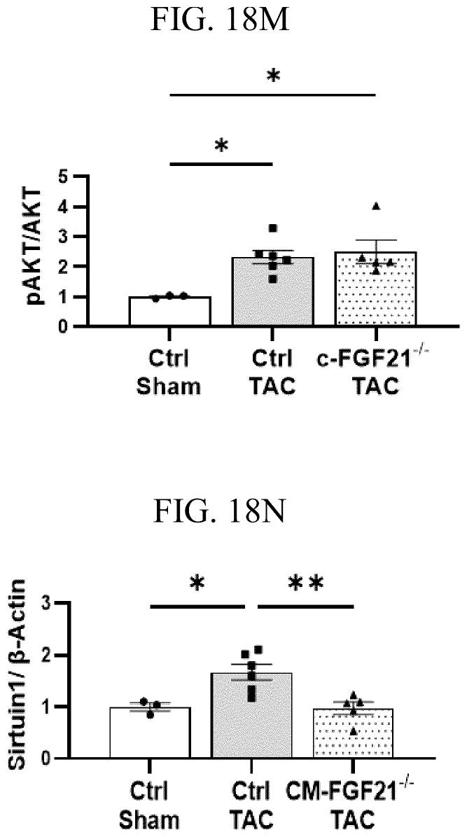

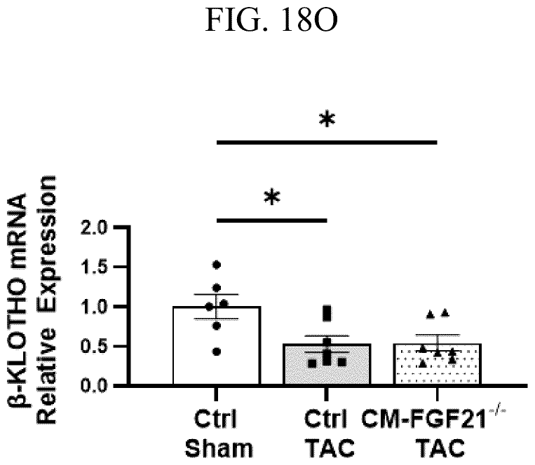

Cardiomyocyte FGF21 is the primary driving force of pressure overload-induced hypertrophy and heart failure. To investigate whether cardiomyocyte FGF21 upregulation per se is involved in the pathophysiology of cardiac hypertrophy with pressure overload, we generated cardiomyocyte-specific FGF21 knockout mice (CM-FGF21−/−) by crossing αMHC-Cre+/− mice with Fgf21f1/f1 mice. Successful FGF21 deletion was confirmed with lower expression of FGF21 in cardiomyocytes (FIG. 17A). We also showed that cardiomyocyte-specific deletion of FGF21 did not affect either hepatic FGF21 (FIG. 17B) or WAT FGF21 mRNA levels (FIG. 17C). Application of TAC in CM-FGF21−/− mice for 8 weeks (FIG. 17D) did not increase cardiac FGF21 expression although there was a statistically non-significant trend of increase in both male and female mice (FIG. 4A, FIG. 17E), despite the elevation of hepatic FGF21 expression (FIG. 17F) and plasma FGF21 levels (FIG. 4B). Importantly, cardiomyocyte FGF21 ablation prevented the onset of cardiac hypertrophy (FIG. 4C) and cardiac dysfunction (FIG. 4D, 4E and Tables 14 and 15). CM-FGF21−/− mice with TAC did not present any sex-specific differences either in hepatic (FIG. 17F) or circulating (FIG. 17G) levels of FGF21 or in hypertrophy (FIG. 17H) and cardiac function (FIG. 17I). Accordingly, CM-FGF21−/− mice with TAC had lower expression of BNP (FIG. 4F) and 3-MHC (FIG. 4G) and less profound cardiomyocyte enlargement (FIGS. 4H and 4I) and myocardial fibrosis (FIGS. 4H and 4J), and fibrosis-related gene expression as shown by lower cardiac mRNA levels of COL1A1, COL3A1 and PSTN (FIGS. 17J-17L). Cardiac expression (FIGS. 18A-18C) and plasma content (FIGS. 18D-18F) of inflammatory cytokines, IL-1α, IL-β, TNFα were markedly decreased in CM-FGF21−/− mice compared to Fgf21f1/f1 mice with TAC. The anti-inflammatory IL-10 cardiac mRNA and plasma levels remained unchanged (FIGS. 18G and 18H). Furthermore, besides lower cardiac FGF21 protein content (FIG. 4K, FIG. 18I), CM-FGF21−/− mice with TAC had lower pAMPK/tAMPK (FIG. 4K, FIG. 18J) and pERK/tERK (FIG. 4K, FIG. 18K), implying inactivation of pro-hypertrophic signaling pathways, compared to control mice with TAC. Notably, cardiac protein abundance of the FGF21 expression regulators, PPARα (FIG. 4K, FIG. 18L) and pAkt/tAkt (FIG. 4K, FIG. 18M), was either partially suppressed or remained elevated in CM-FGF21−/− mice. On the other hand, cardiac SIRT1 expression was restored to normal levels in CM-FGF21−/− mice (FIG. 4K, FIG. 18N). Moreover, cardiac FGFR1 expression was not upregulated in CM-FGF21−/− mice with TAC (FIG. 3L) as we observed in HEP-FGF21−/− mice (FIG. 3P). However, the expression of 0-KLOTHO mRNA was suppressed by ˜50% in both control and CM-FGF21−/− mice with TAC (FIG. 18O). Conclusively, despite the increase of hepatocyte-derived FGF21, cardiomyocyte-specific ablation of Fgf21 recapitulates the cardioprotective effects that were observed in the HEP-FGF21−/− mice although signaling that promotes FGF21 expression is not completely blocked. Thus, stimulation of cardiomyocyte FGF21 expression with pressure overload is a critical molecular event that accounts for cardiac hypertrophy and heart failure.

### Example 10

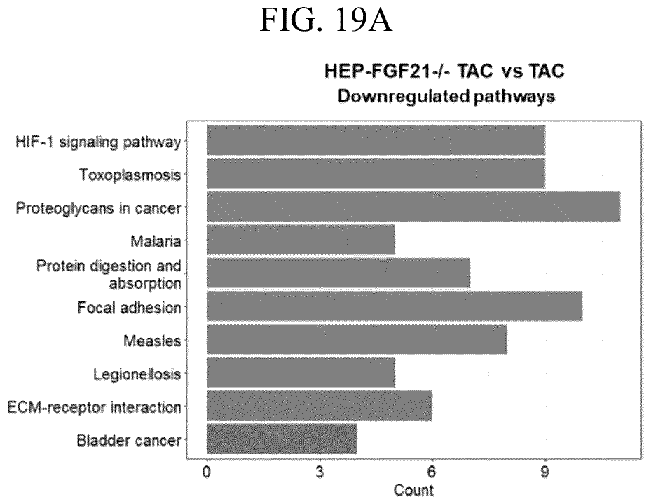

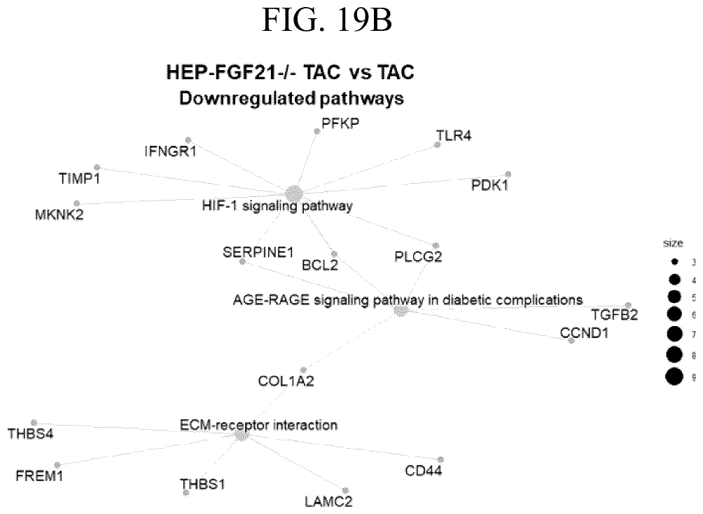

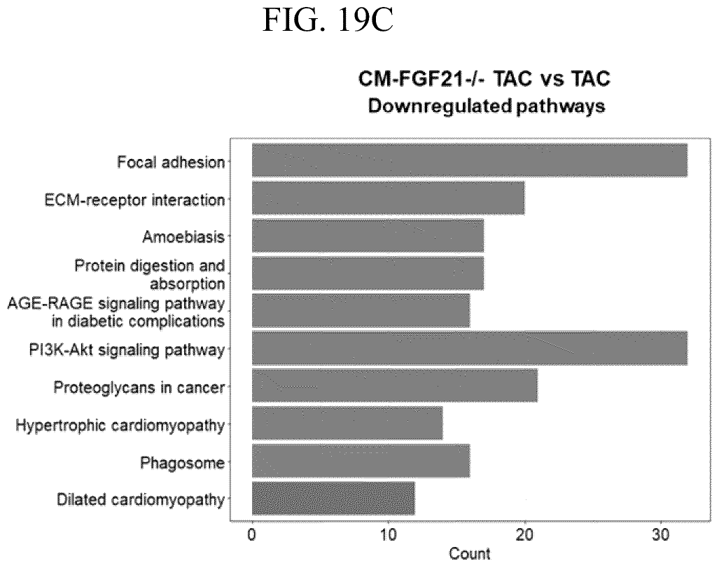

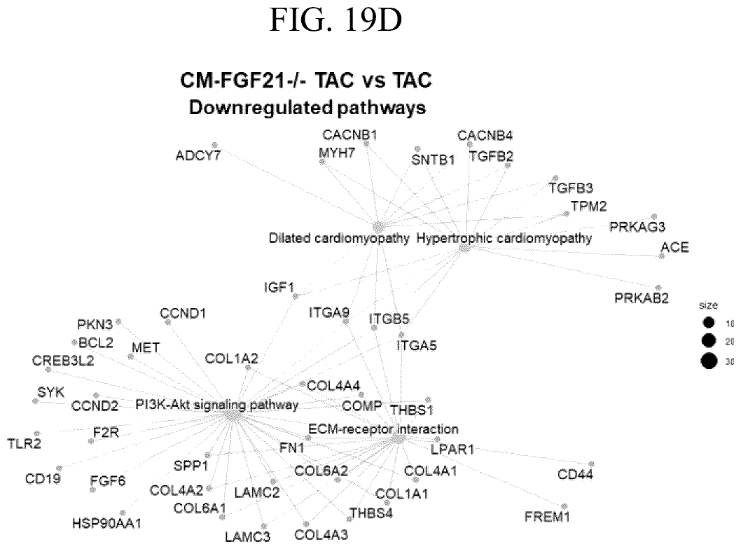

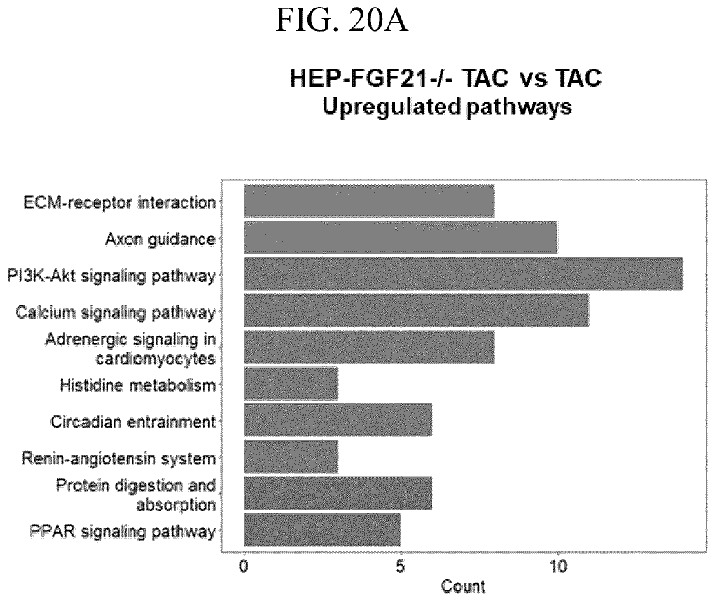

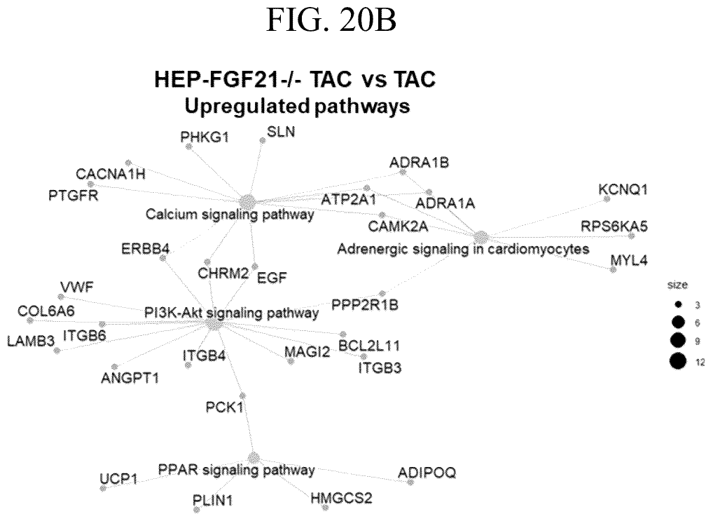

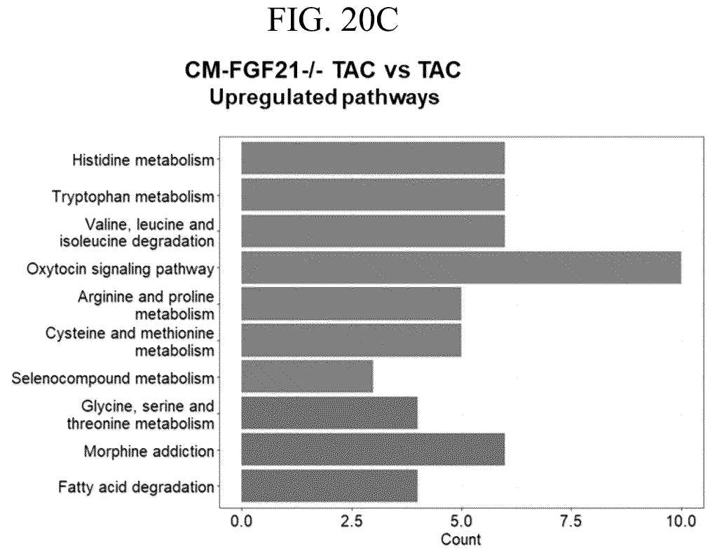

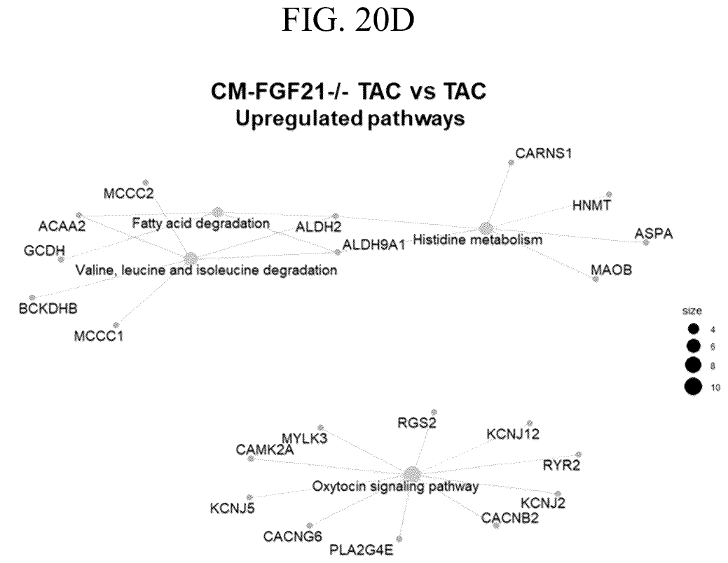

Inhibition of FGF21 signaling reversed pro-hypertrophic myocardial transcriptome, metabolome and lipidome profiles—To identify common pathways that are altered in both HEP-FGF21−/− and CM-FGF21−/− and may be involved in the cardioprotective effect of FGF21 suppression in either hepatocytes or cardiomyocytes, we compared cardiac transcriptome (RNAseq), lipidome and metabolome profiles between HEP-FGF21−/− and CM-FGF21−/− mice with TAC against control mice with TAC. These analyses identified distinct transcriptome profiles in HEP-FGF21−/− mice subjected to TAC with 721 differentially expressed genes, (403 upregulated, 318 downregulated) (FIGS. 5A-5B). Similarly, CM-FGF21−/− mice exhibited differential expression of 1155 genes, with 519 being upregulated and 636 downregulated compared to control mice with TAC (FIGS. 5A-5B). Among all genes that had differential expression, 406 had the same changes in both HEP-FGF21−/− and CM-FGF21−/− mice with 202 genes being upregulated and 204 genes downregulated (FIGS. 5A, 5B and Table 16, 17). KEGG pathway analysis indicated that the induction of the ECM receptor interaction-related gene expression program was suppressed both in HEP-FGF21−/− and CM-FGF21−/− mice (FIG. 5C, FIGS. 19A-19D). On the other hand, pathways such as PI3K-AKT, hypertrophic cardiomyopathy, and dilated cardiomyopathy pathways that are induced in control mice with TAC, were surprisingly not reversed in HEP-FGF21−/− (FIGS. 20A and 20B) although they were attenuated in CM-FGF21−/− mice (FIGS. 20C and 20D). Notably, expression of genes related to BCAA catabolism and FA degradation was increased in CM-FGF21−/− mice (FIGS. 20C and 20D), implying restoration of these metabolic pathways to normal levels.

Metabolomic analysis revealed higher levels of pyruvate and fumarate (Krebs cycle metabolite) in HEP-FGF21−/− (FIG. 5K, Table 18) and CM-FGF21−/− mice (FIG. 5K, Table 19) 8 weeks post-TAC. Moreover, HEP-FGF21−/− mice have higher citrate, NAD+, and ATP levels, suggesting improvement in cardiac energetics (FIG. 5K, Table 18).

Cardiac lipidome profiling showed that the broad increase of various species of phospholipids (PC, PE, PS) which was observed in control mice with TAC for 8 weeks, was mostly reversed in HEP-FGF21−/− (FIG. 5K, Table 20) and CM-FGF21−/− (FIG. 5K, Table 21) mice with TAC, particularly for PC 17:1/18:2, PE 16:1/18:1, PE 18:0/18:2, PE 22:0/20:4, PE 22:0/22:6, and PS 18:4/22:4. These analyses collectively suggest that deletion of FGF21 either in hepatocytes or cardiomyocytes attenuates the expression of a subset of genes that are associated with cardiomyopathy and lowers biosynthesis of phospholipids likely because there is less need for membrane biosynthesis due to the reversal of the hypertrophic program.

### Example 11

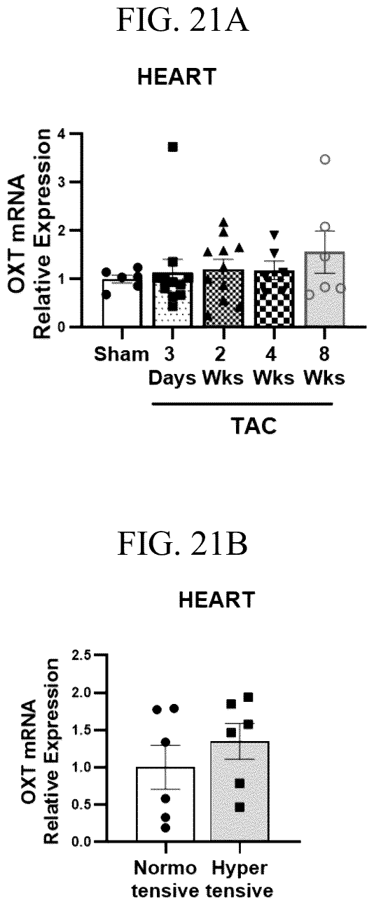

The cardioprotective effect of FGF21 inhibition is associated with increased oxytocin signaling in the heart. Strikingly, the expression of genes which encode for proteins that are involved in the cardioprotective oxytocin signaling was increased in both HEP-FGF21−/− and CM-FGF21−/− mice (FIG. 5D). Interestingly, components of this pathway had the highest representation among the upregulated pathways in CM-FGF21−/− mice with TAC compared to control mice with TAC (FIGS. 20C and 20D). As protection from oxytocin signaling has been attributed to either a cardiac autocrine pathway or endocrine effects of oxytocin that is produced and released by the hypothalamus, we measured oxytocin expression in both organs during progression of LVH development. Our analyses revealed that cardiac oxytocin expression does not change at any time point in mice with TAC (FIG. 21A) or in hearts of humans with hypertension (FIG. 21), thus excluding potential autocrine effects. Conversely, oxytocin expression in the hypothalamus is significantly suppressed upon maximal release of FGF21 in the circulation (3 days post-TAC) and remains at low levels until 8 weeks post-TAC (FIG. 5E). Notably, oxytocin expression in the hypothalamus is not suppressed in HEP-FGF21−/− mice that have low levels of systemic FGF21 and are protected from pressure overload-induced LVH (FIG. 5E). On the cardiac side, the levels of oxytocin receptor (OXT-R) mRNA in wild type hearts are increased at the early stage of TAC (3 days), prior to the development of pathological hypertrophy, and gradually return to normal levels by 8 weeks post-TAC (FIG. 5F). Measurement of OXT-R protein levels in the hearts of wild type and HEP-FGF21−/− mice 8 weeks post-TAC revealed a 55% suppression in wild type mice, which did not occur in the protected HEP-FGF21−/− mice (FIGS. 5G and 5H). Thus, inhibition of hepatic FGF21 expression in mice with TAC, maintains normal levels of oxytocin expression in the hypothalamus and OXT-R in the heart.

To investigate if oxytocin can reverse cardiomyocyte hypertrophy, we treated primary cardiomyocytes from adult mice with a combination of FGF21 and increasing levels of oxytocin. Analysis of the cardiomyocyte area showed that oxytocin attenuated the pro-hypertrophic effect of FGF21 (FIGS. 5I and 5J). Thus, suppression of oxytocin signaling can exacerbate the effect of FGF21 in promoting LVH.

### Example 12

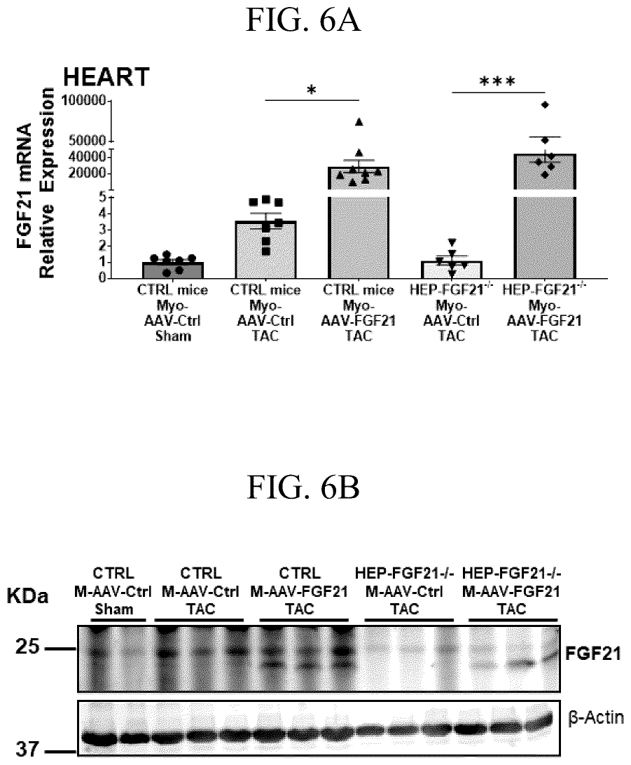

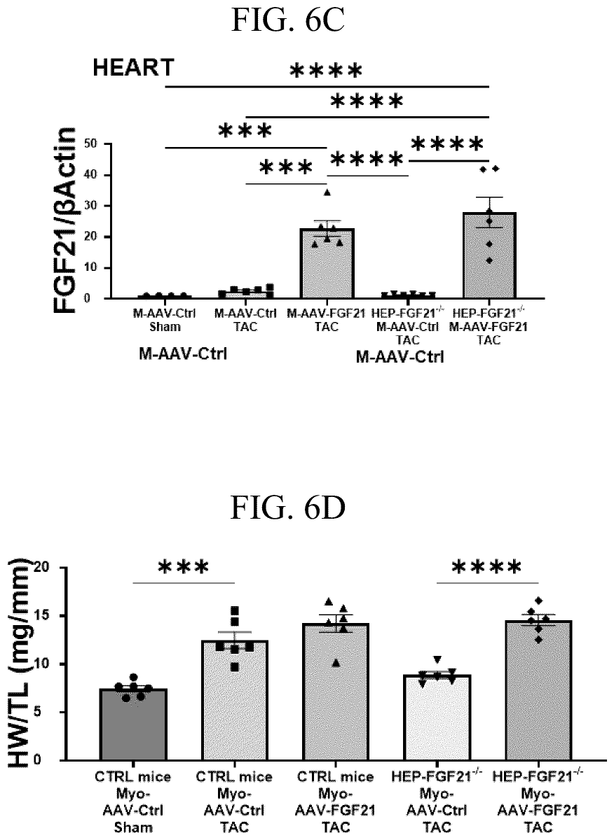

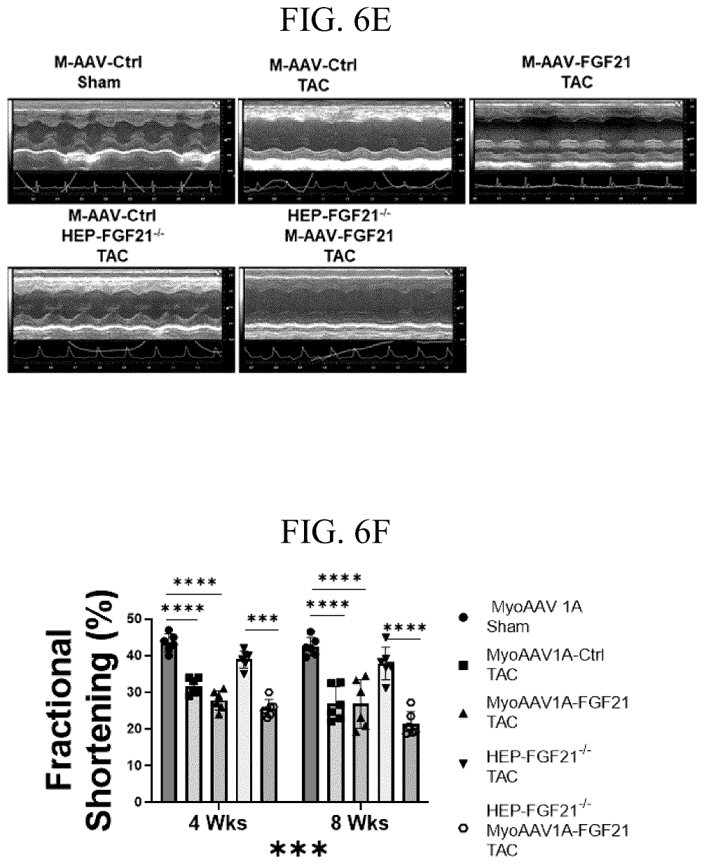

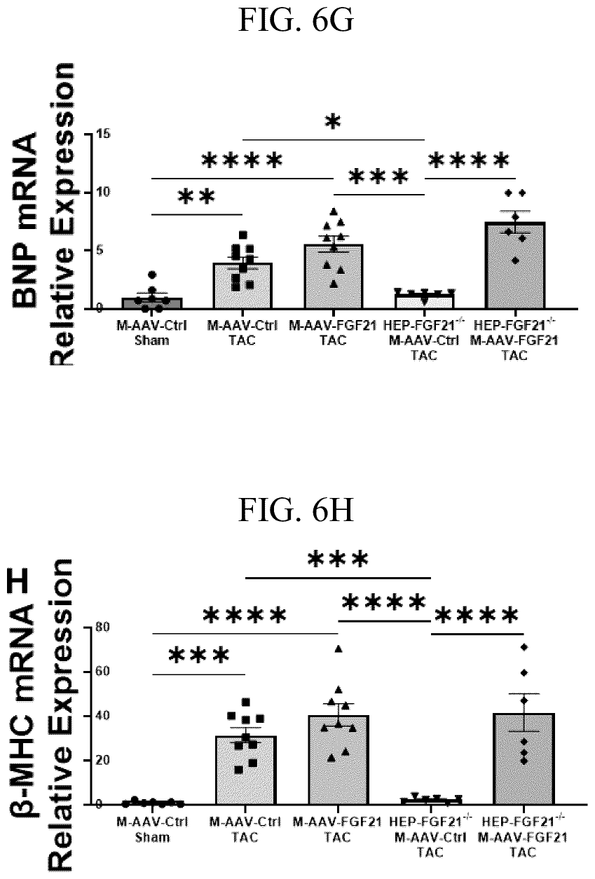

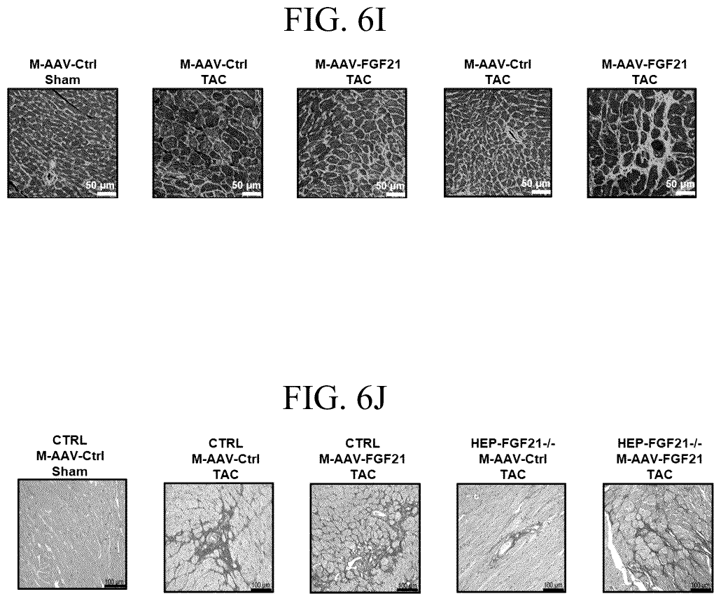

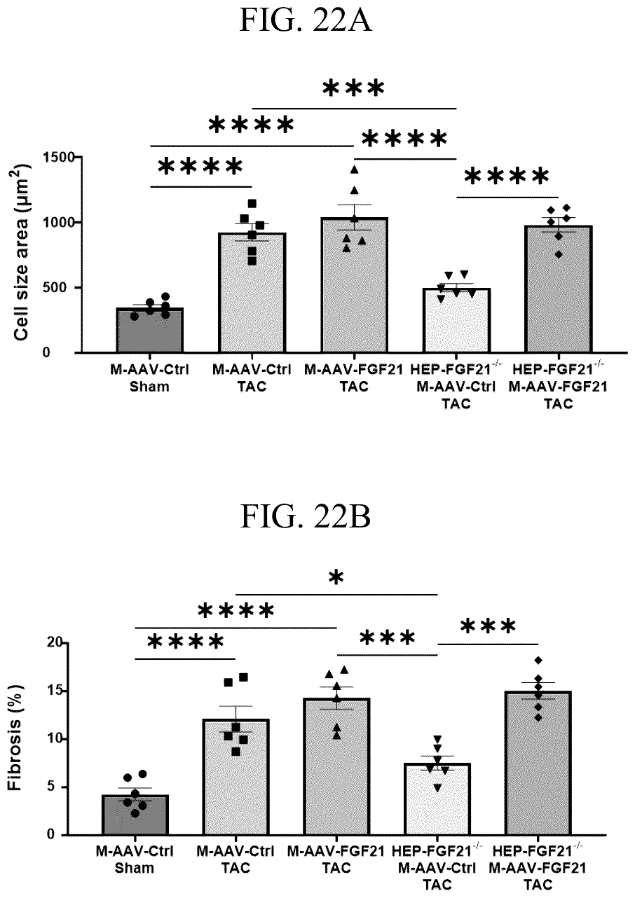

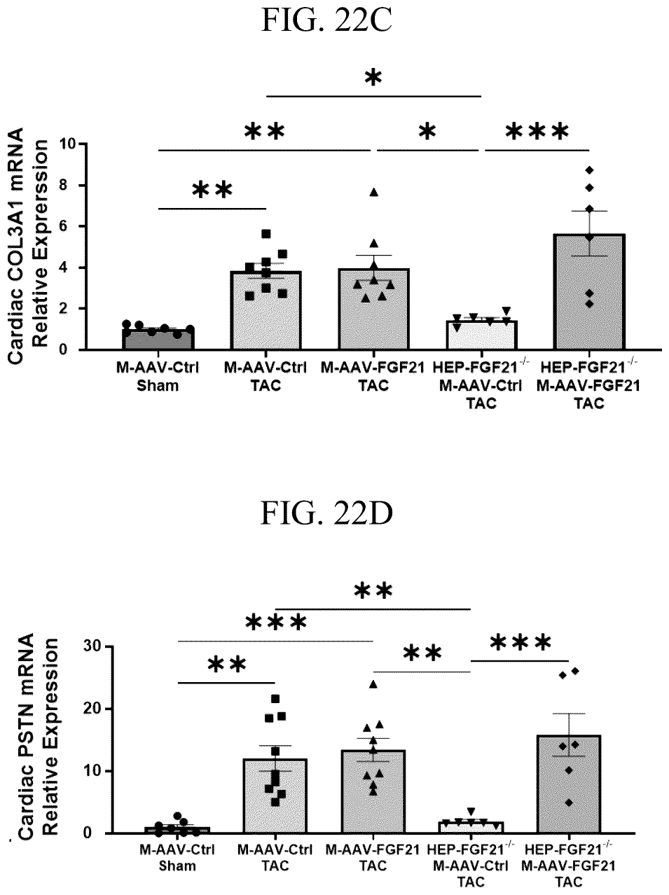

The cardioprotective effect of hepatic FGF21 deletion is abolished upon cardiomyocyte FGF21 activation. To further explore if cardiomyocyte FGF21 per se is a main cause for the development of pathological hypertrophy with pressure overload, we induced cardiomyocyte FGF21 overexpression in mice that do not express hepatic FGF21 (HEP-FGF21−/−) by injecting them and control FGF21f1/f1 mice with 1×1012 particles/mouse of a cardiotropic Myo-AAV91A 51 that encodes for mouse FGF21 under the control of the cardiomyocyte-specific cardiac troponin T (cTnT) promoter (Myo-AAV91A-cTnT-FGF21). Four weeks post-AAV injection, we subjected HEP-FGF21−/− and FGF21f1/f1 mice to TAC and monitored them for 8 additional weeks. Control HEP-FGF21−/− and FGF21f1/f1 mice were injected with empty Myo-AAV91A-cTnT and TAC was applied. FGF21f1/f1 that were injected with empty Myo-AAV91A-cTnT and subjected to sham surgery were also used as an additional control group. We first confirmed that injection of Myo-AAV91A-cTnT-FGF21 increased cardiac FGF21 mRNA (FIG. 6A) and protein levels (FIGS. 6B and 6C) compared to FGF21f1/f1 mice injected with empty Myo-AAV91A-cTnT. As expected, control FGF21f1/f1 mice that were injected with Myo-AAV91A-cTnT-FGF21 and subjected to TAC developed cardiac hypertrophy (FIG. 6D) and cardiac dysfunction (FIGS. 6E, 6F and Tables 22, 23) compared to HEP-FGF21−/− mice injected with control virus and subjected to TAC or the FGF21f1/f1 mice injected with control Myo-AAV91A-cTnT and subjected to sham surgery. Strikingly, however, the protective effect of hepatocyte Fgf21 deletion against TAC-induced hypertrophy (FIG. 6D) and cardiac dysfunction (FIGS. 6E, 6F, Tables 22, 23) was abolished in HEP-FGF21−/− mice that cardiac FGF21 expression was stimulated with Myo-AAV91A-cTnT-FGF21. Negation of the protective effect of hepatocyte FGF21 ablation when cardiac FGF21 expression was stimulated was also reflected on higher expression of heart failure markers (FIGS. 6G and 6H), cardiomyocyte enlargement (FIG. 61, FIG. 22A), increased myocardial fibrosis (FIG. 6J, FIG. 22B) and higher fibrosis-related gene expression (FIG. 22C, 22D). In summary, stimulation of cardiomyocyte FGF21 expression alone leads to pressure overload-induced hypertrophy even in the absence of hepatic FGF21 signaling.

### Example 13

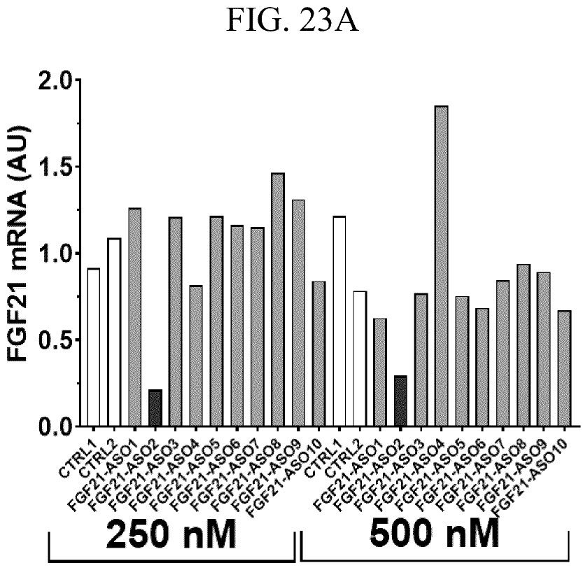

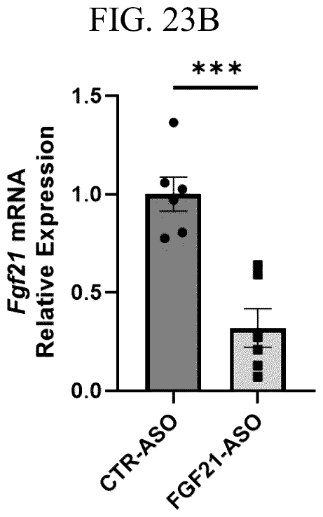

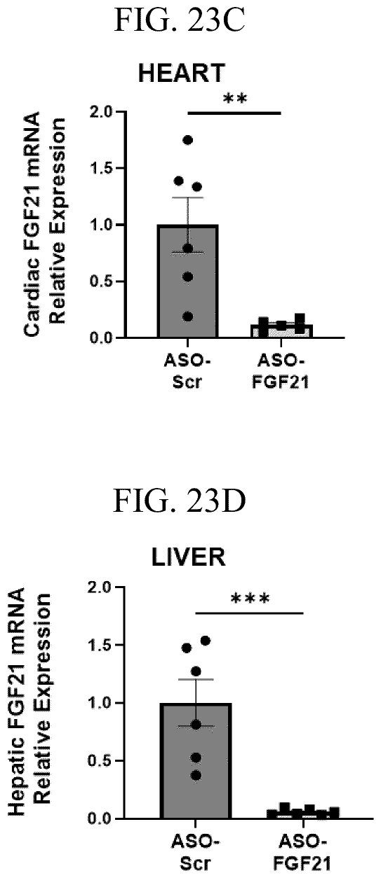

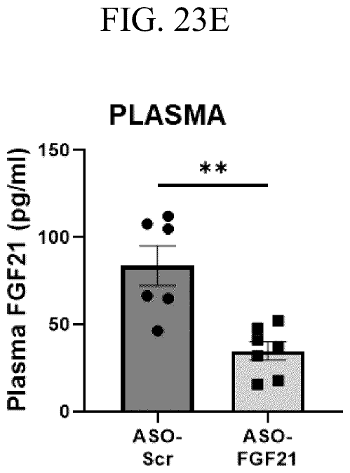

ASO-mediated inhibition of FGF21 is cardioprotective against TAC-induced heart failure—To further explore the translational perspective of the therapeutic effects of FGF21 inhibition, we developed anti-sense oligonucleotides that target FGF21 (ASO-FGF21). To this end, we performed in vitro and in vivo evaluation of 10 prospective ASOs (Table 24) that were designed against mouse FGF21 to identify the lead candidate for in vivo studies. First, we performed an initial screening that involved treatment of a mouse cardiac muscle cell line (HL-1) with either 250 nM or 500 nM of 10 different ASO-FGF21 or control scrambled ASO (ASO-scr) for 48 hours and checked FGF21 expression (FIG. 23A). Among the 10 tested ASOs, ASO-FGF21-[2](5′-GGUGACGGGGGAAAGUAGGU 3′) incurred suppression of FGF21 by 70% at the concentration of 250 nM. ASO-FGF21-[2] was further validated for successful suppression of FGF21 expression in HL-1 cells (FIG. 23B). The selected ASO-FGF21 and ASO-scr were chemically modified for increased stability when applied in vivo with central gap segment comprising ten 2′-deoxynucleosides and flanked on the 5′ and 3′ wings by five 2′-MOE modified nucleosides. All inter-nucleotide linkages are phosphorothioate linkages. The capacity of the selected ASO-FGF21 to suppress FGF21 expression in vivo was tested in healthy wild type mice via retroorbital injection (10 mg/kg BW) that was applied weekly for 4 weeks. After 4 weeks of treatment, we observed significant suppression of cardiac (88%, FIG. 23C) and hepatic (94%, FIG. 23D) FGF21 mRNA levels, as well as in plasma FGF21 levels (48%, FIG. 23E).

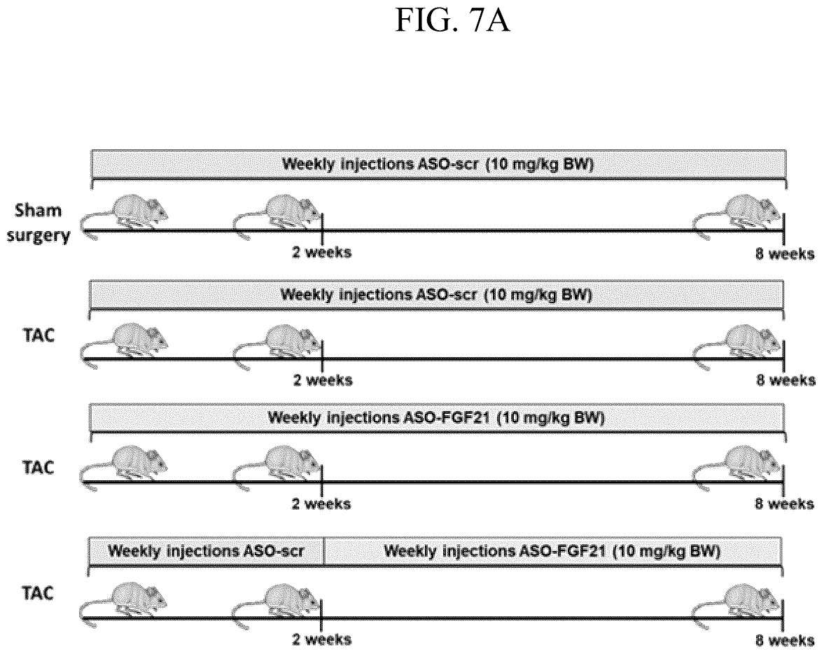

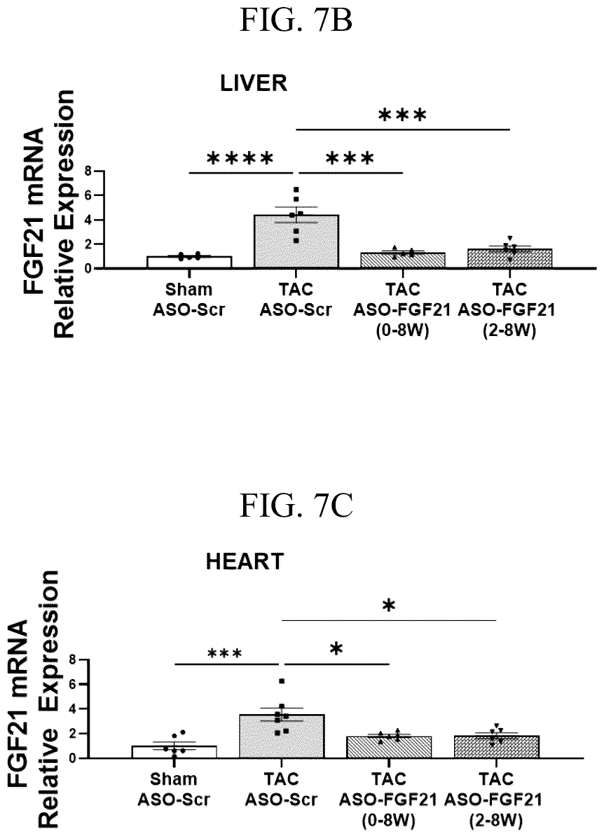

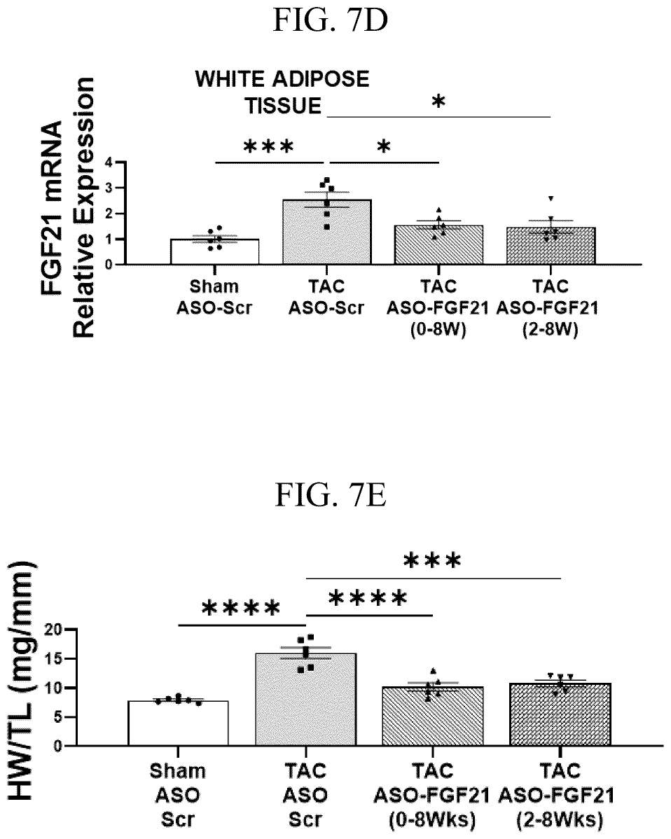

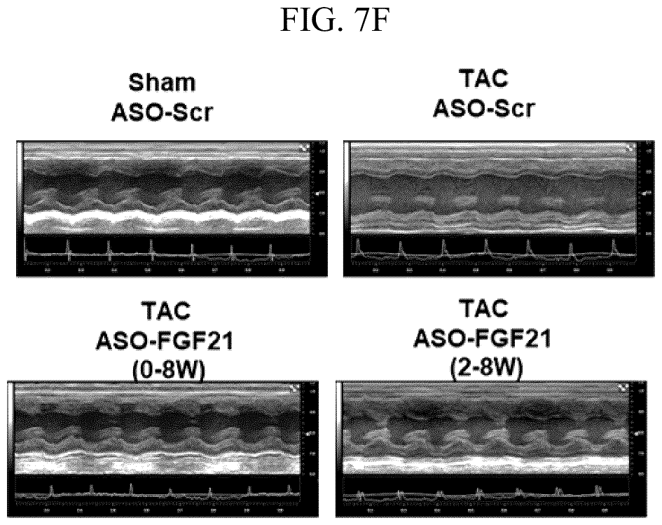

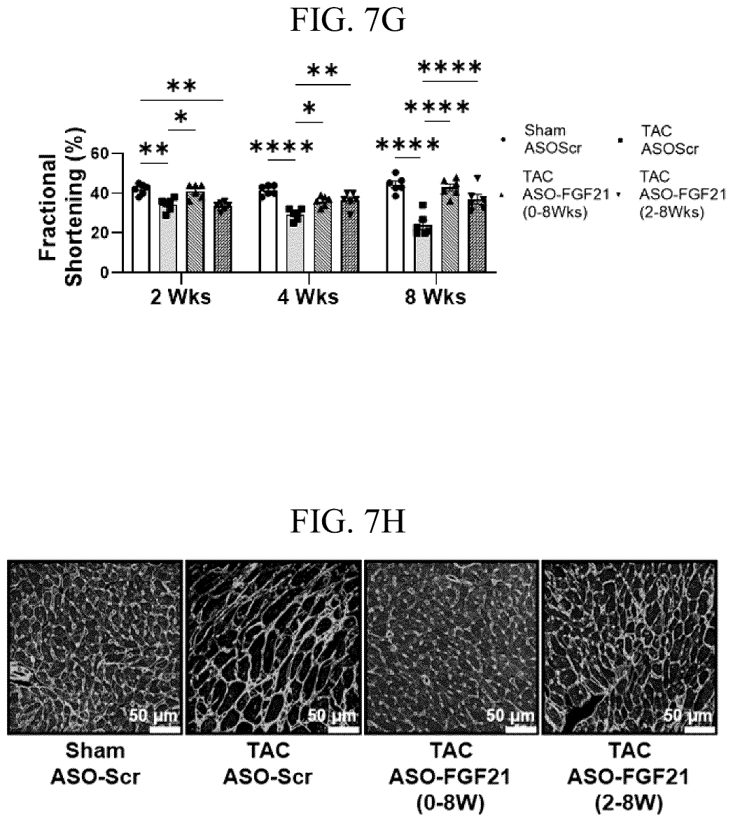

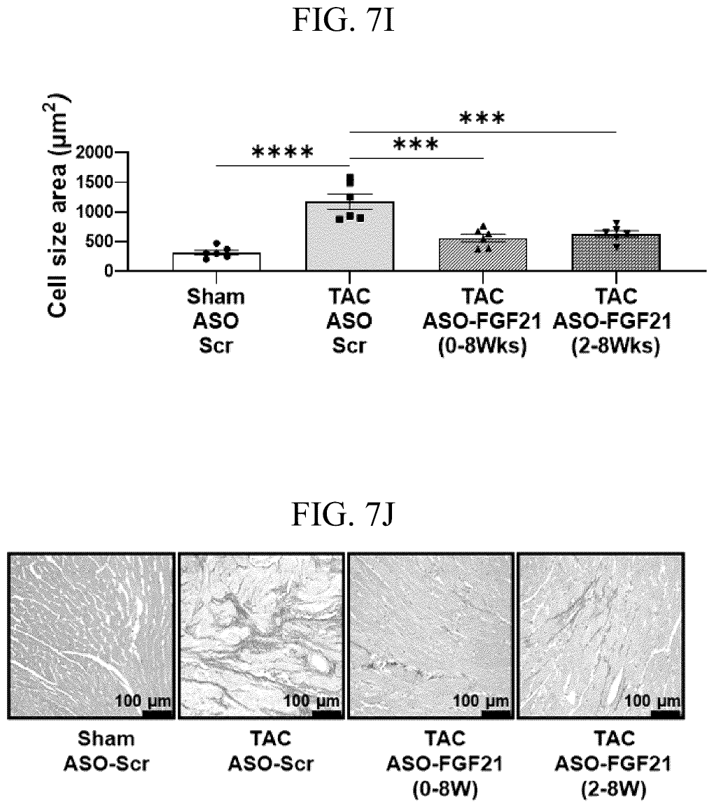

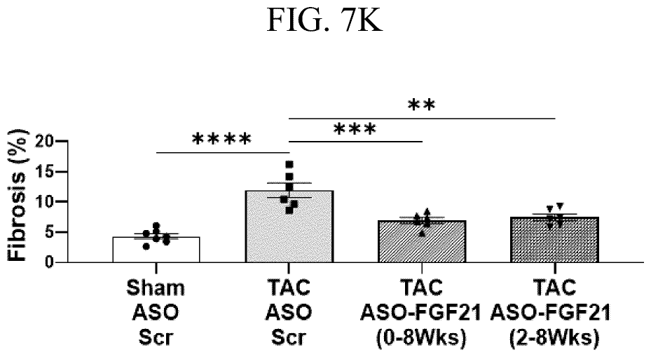

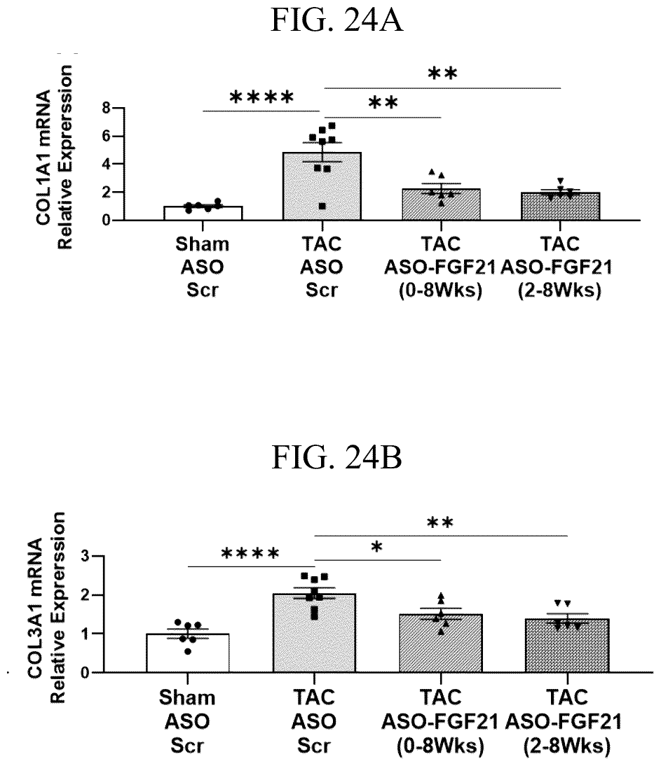

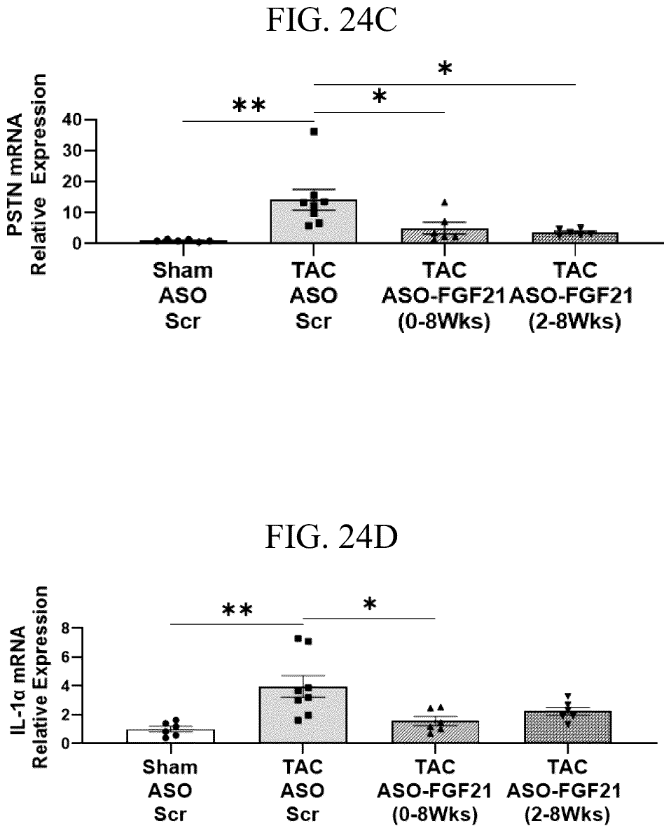

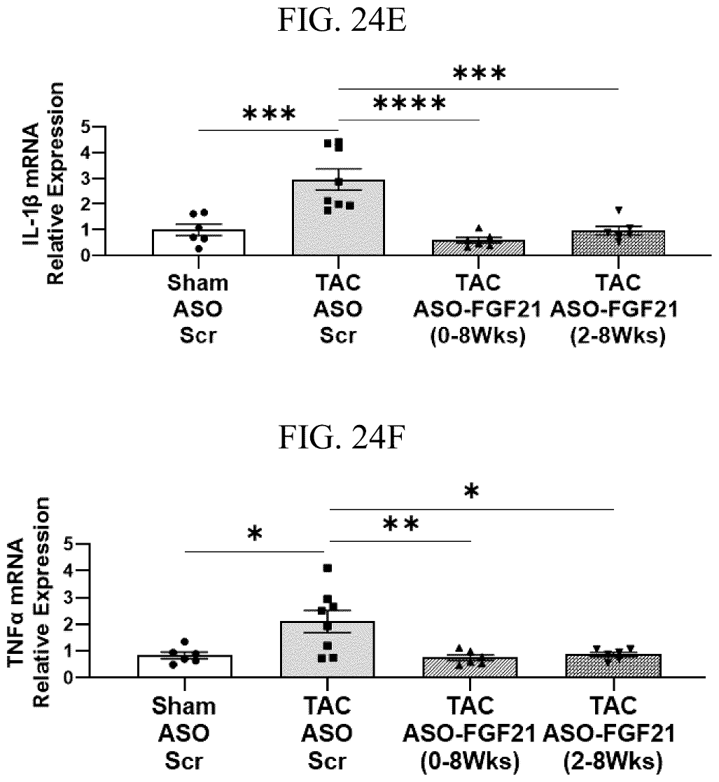

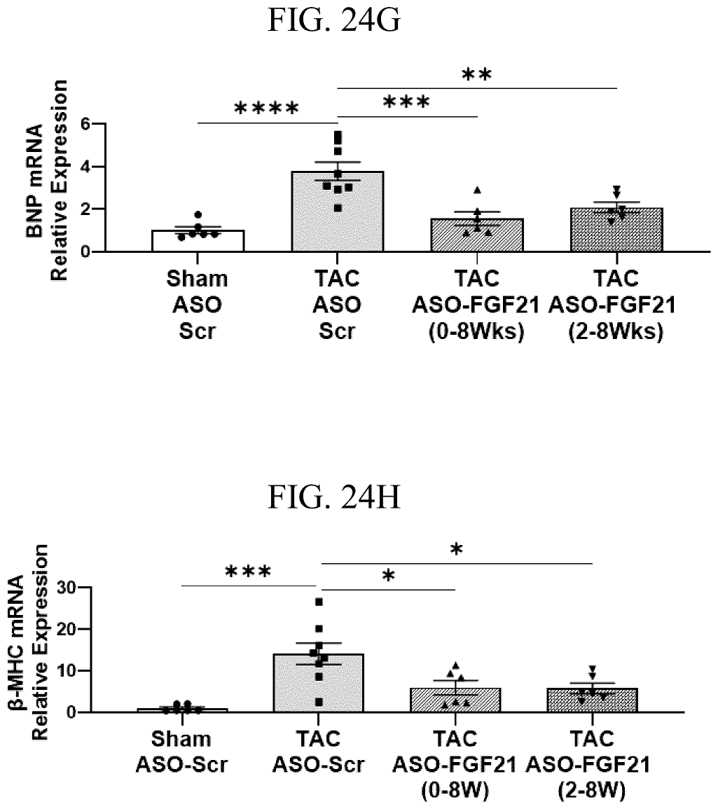

The most effective ASO-FGF21 was tested in vivo in wild type mice with TAC or control surgery. For a group of mice, the ASO-FGF21 treatment (10 mg/kg BW weekly injections) started the next day after TAC and lasted 8 weeks (FIG. 7A). Another group of wild type mice with TAC was injected with control ASO-scr for the first 2 weeks post-TAC. At the 2-week time point, when pathological hypertrophy is observed (FIG. 1D), we performed weekly injections with ASO-FGF21 for 6 more weeks (FIG. 7A). Control wild type mice with either sham surgery or TAC were treated with ASO-scr for 8 weeks (FIG. 7A). The ASO-FGF21 treatment lowered expression of FGF21 in the liver (69% for 0-8 weeks and 63% for 2-8 weeks, FIG. 7B), heart (49% for 0-8 weeks and 48% for 2-8 weeks, FIG. 7C), and WAT (38% for 0-8 weeks and 41% for 2-8 weeks, FIG. 7D). In accordance with the genetic FGF21 loss of function models, ASO-FGF21 alleviated cardiac hypertrophy (FIG. 7E), and improved cardiac function (FIGS. 7F, 7G, Tables 25, 26) in both treatment protocols (0-8 weeks and 2-8 weeks). Accordingly, cardiomyocyte enlargement (FIGS. 7H and 7I), myocardial fibrosis (FIGS. 7J and 7K), cardiac fibrosis-related (FIGS. 24A-24C) and inflammation-related gene expression (FIGS. 24D-24F) were attenuated. Furthermore, the ASO-FGF21 treatment reduced the expression levels of heart failure markers BNP (FIG. 24G) and β-MHC (FIG. 24H). In conclusion, systemic or cardiomyocyte-specific inhibition of FGF21 holds therapeutic value for LVH and cardiac dysfunction that are induced by increased afterload.

### Methods

### Human Population Study

We enrolled consecutive subjects, aged >50 years with and without essential hypertension and no indications of cardiovascular disease. The diagnosis of hypertension was based on the recommendations of the European Society of Hypertension/European Society of Cardiology. We excluded patients with history of coronary artery disease, moderate or severe valvular heart disease, cardiomyopathy, cerebrovascular, liver or renal disease, ejection fraction <55%; history of drug or alcohol abuse; any inflammatory or other infectious disease during the last 6 months; thyroid gland disease. Vascular or neoplastic conditions were also ruled out. A full echocardiographic study was performed in all subjects using a Vivid 7 (General Electric, Horten, Norway) ultrasound device according to the recommendations of the European Association of Cardiovascular Imaging and American Society of Echocardiography. Blood samples were collected from all participants and the serum was separated by centrifugation at 4,000 rpm for 10 minutes. The serum was then stored at −80° C. Serum FGF21 levels were measured in duplicates, using the BioVendor Human FGF-21 ELISA kit according to the manufacturer's protocol. The study was conducted in accordance with the Declaration of Helsinki, the protocol was approved by the Hospital Ethics Committee, and patients gave written informed consent to their participation in the study.

Human Myocardial Tissue Acquisition—Myocardial tissue from humans was obtained through the National Disease Research Interchange (NDRI). Tissue samples were collected from the LV or RV or apex and were snap frozen before storing at −80° C.

### Animal Care

All procedures involving animals were approved by the Institutional Animal Care and Use Committees at the University of Cincinnati and carried out in accordance with the NIH guidelines. All animals used were 8-12 weeks old at the beginning of the experiments. We used both male and female mice in our study. The mice were maintained under appropriate barrier conditions in a 12h light-dark cycle and received food and water ad libitum. No mice were excluded from the study unless their wellness was compromised and a recommendation for euthanasia was made by the animal facility supervisors.

### Genetic Mouse Models

We generated hepatocyte-specific FGF21 knockout mice (HEP-FGF21−/−) by crossing Alb-Cre+/− mice with FGF21f1/f1 mice and cardiomyocyte-specific FGF21 knockout mice (CM-FGF21−/−) by crossing αMHC-Cre+/− mice with FGF21f1/f1 mice. We also generated skeletal muscle specific FGF21−/− mice (MLC-FGF21−/−) by crossing MLC-Cre+/− mice with FGF21f1/f1 mice. All HEP-FGF21−/−, CM-FGF21−/− and MLC-FGF21−/− progeny were viable, fertile, reproduced at expected Mendelian ratios, and showed no pathological phenotypes at baseline.

### Transverse Aortic Constriction

Mice were anesthetized with 3% isoflurane and intubated with 18-gauge catheter. Anesthesia was maintained during surgery with a mouse ventilator providing 2% isoflurane. The buprenorphine-extended release and meloxicam-extended release were administered preemptively at 2 mg/kg BW and 1 mg/kg BW respectively to reduce pain. A thoracotomy was performed, and the transverse aorta was isolated and tied around 27-gauge needle, which was removed to generate the desired constriction. The incision to the rib cage was closed using a 6.0 silk suture (AD Surgical) and the skin wound was closed with a 5.0 silk suture. (AD Surgical). The sham procedure was identical except that aortic arch was not constricted.

### Partial Inferior Vena Cava Ligation

Wild-type C57BL/6 male mice were purchased from Jackson Labs. At 8 weeks of age mice underwent partial ligation of the inferior vena cava via the Mouse Cardiovascular Phenotyping Core at Washington University School of Medicine. The supr-hepatic IVC was exposed through a subxiphoid incision, then ligated to achieve a nearly 70% reduction of the IVC diameter as previously described101. After 12 weeks the mice were anesthetized with CO2, followed by cervical dislocation. Livers were perfused with PBS via the portal vein and snap-frozen in liquid nitrogen102. Food and water were provided ad libitum. All animal studies were performed according to the Washington University in St Louis Institutional Animal Care and Use Committee.

### Echocardiography

Ultrasound examination was performed using Vevo F2 (VisualSonics F2 Ultrasound, Visualsonics). Mice were lightly anesthetized with 1.5% isoflurane. Hair was removed from the anterior chest using chemical remover, and the animals were placed on a warming pad. Ultrasound gel was applied to the chest. Care was taken to maintain adequate contact while avoiding excessive pressure on the chest. Two-dimensional echocardiographic images were recorded in a digital format. Complete 2-D short or long axis and doppler ultrasound examination (where applicable) were performed using multiple views and analyzed short-axis for M-mode and long axis for B-mode images by LV trace. To measure myocardial strain, speckle-tracking probe was applied to the long axis B-mode images using Vevo strain software.

To measure transaortic pressure gradients to assess success of TAC surgery, echocardiogram was performed at 1 week after surgery for all sham and TAC group using an M250D probe to obtain an image of the complete descending aorta. Blood velocity across the descending aorta was measured and extrapolated to pressure using Visual Sonics analysis software. Animals with peak transaortic pressure gradients below 40 mmHg were excluded from analysis. Animals were euthanized at the end of each study, and gravimetric for heart and tibia length were assessed before organ preservation.

### Tissue Histological Sectioning and Staining

Cross sections from the middle region of the left ventricle were taken immediately upon removal from the heart and were fixed in formalin for 24 hours (followed by storage in 70% ethanol at 4° C. before embedding and sectioning). Heart sections at a thickness of 5 m were stained with Alexa Fluor 488-conjugated wheat germ agglutinin (WGA) overnight at 4° C. at 1:1000 dilution in a humidified chamber. Sections were washed three times for 5 min with PBS followed by mounting with coverslips using Fluoromount-G mounting medium containing 4′,6-diamidino-2-phenylindole (DAPI) nuclear stain (Southern Biotech). A Leica Stellaris 8 confocal (Leica Microsystems, Germany) was used to record images. The WGA was utilized for measurement of myocyte cross-sectional area at 25 myocytes were assessed per heart using Image J software (NIH). Picrosirius Red Staining (PSR) of collagen fibers was used to assess left ventricular interstitial fibrosis by using Leica DMi8 widefield fluorescence microscope (Leica Microsystems, Germany) followed by thresholding method using Image J software. The border areas of the slices, where cells are naturally hypertrophic and fibrotic, were excluded from imaging. Myocyte area quantification was conducted only on cells with a visible nucleus. Representative images were chosen based on the mean or average values (n=6 hearts per group).

### Adult Mouse Cardiomyocyte Isolation

Adult mouse cardiomyocytes (ACMs) were isolated from the heart ventricles of 8-12 weeks old mice. Briefly, mice were anesthetized using pentobarbital (19 mg/mL) and intraperitoneally injected with 0.5 mL of heparin (100 IU/mL in PBS). The rib cage was opened, and the heart was exposed, excised, and immediately cannulated via the aorta.

The heart was perfused with perfusion buffer (120.4 mM NaCl, 14.7 mM KCl, 0.6 mM NaH₂PO₄, 1.2 mM MgSO₄, 10 mM HEPES, 4.6 mM NaHCO₃, 30 mM Taurine, 10 mM BDM, 5.5 mM glucose; pH 7.0) at 4 mL/min for 4 minutes, followed by myocyte digestion buffer containing 19,250 units of collagenase type II (Worthington), 5-6 mg of trypsin, and 1 mM CaCl₂) for 7 minutes. Ventricles were gently teared in small pieces, perfusion buffer containing 5 mg/ml BSA and 0.125 mM CaCl₂) was added and filtered with 100 m nylon strainers. The filtrate was pelleted by gravity for 5 mins, centrifuged for 30 sec at 700 rpm and the pellet resuspended in perfusion buffer containing 5 mg/ml BSA and 0.225 mM CaCl₂). The cells were pelleted by gravity for 10 min, centrifuged for 30 sec at 700 rpm and the pellet was resuspended in perfusion buffer containing 5 mg/ml BSA and 0.525 mM CaCl₂). The cells were pelleted by gravity for 10 min, centrifuged for 30 sec at 700 rpm and the pellet was resuspended in perfusion buffer containing 5 mg/ml BSA and 1.025 mM CaCl₂). Only cell preparations with >90% rod-shaped, viable cardiomyocytes were used for experiments. Laminin-coated culture plates (10 μg/mL for 6 hours at room temperature) were used to plate the ACMs, which were subsequently treated with rm-FGF21 with 10 nM concentration (R&D, 8409-Catalog #FG-025/CF) and/or oxytocin with 10 nM and 100 nM concentration (peptide science, catalog #31-120-PS), as applicable, for 24 hours for microscopy or RNA isolation.

### Cell Culture

The human ventricular cardiomyocyte cell line (AC16) was maintained in complete DMEM/F-12 medium supplemented with 10% FBS and 1% penicillin/streptomycin mix at 37° C. and 5% CO2. AC16 cells were treated with rh-FGF21 (R&D Systems, catalog #2539-FG-025/CF) at 10 nM concentration for 24 hours before cells were harvested for mRNA and protein purification.

For ASO-FGF21 screening, HL-1 (mouse cardiomyocyte cell line) cells were grown in the fibronectin and gelatin coated culture plate with Claycomb basal medium supplemented with 10% FBS (EMD Milipore, Catalog #TMS-016-B), 10 mM norepinephrine (Sigma, catalog #A0937), 200 mM L-Glutamine (TMS-002-C), 1% penicillin/streptomycin (EMD Millipore, Catalog #TMS-AB2-C). The medium was changing approximately every 24 hours. The cells were grown at 37° C. and 5% CO2. HL-1 cells were treated with 250 nM concentration of ASOs (Creative Biolabs Gene Therapy) using lipofectamine 2000 transfection reagent (Thermofischer, Catalog #11668019) for 48 hrs before cells were harvested for mRNA purification.

### Generation of Myo-AAV91A-cTnT-FGF21 Virus

The cardiotropic Myo-AAV91A-cTnT-FGF21 virus was made as previously described103 with the pAV-cTNT plasmid (#PM10013, Vigene Biosciences). The Fgf21 cDNA sequence was obtained from white adipose tissue cDNA and was cloned into the EcoRI and NheI sites of the pAV-cTNT plasmid. The construct was confirmed by DNA sequencing. pAV-cTNT-tdTomato was used as the control. For Myo-AAV91A generation, AAVpro 293T cells (#632273, Takara Bio) were plated in 15 cm dishes. Within 24 hours, cells were transfected with 10.5 ug of pHelper plasmid, 5.25 ug of pRep/Cap plasmid (Myo-AAV91A), and 5.25 ug of gene of interest plasmid using PEI MAX MW 40K (#24765-1, Polysciences). Four days after transfection, Myo-AAV91A was collected from cells and media, and purified by ultracentrifuge with iodixanol gradient. After the purification, buffer was changed to PBS with Amicon Ultra filters (Millipore Sigma). Virus titer was determined by qPCR to calculate genome copies (GC)/ml. The Myo-AAV91A was saved at −80° C. until injection.

### RNA Purification and Gene Expression Analysis

Frozen heart tissue was homogenized on ice with 3-4 ceramic beads and 1 mL cold TRIzol lysis reagent (ThermoFisher 15596026) in a bead homogenizer (Omni International). For cells, 1 mL cold TRIzol lysis reagent (ThermoFisher 15596026) was added to cell pellets. DNase-treated RNA was used for cDNA synthesis using the High-Capacity cDNA Reverse Transcription Kit (Applied Biosystems 4368814) from 500 ng of DNase-treated RNA. Quantitative real-time PCR was performed with the SYBR Select Master Mix (Applied Biosystems 4472903). Incorporation of the SYBR green dye into the PCR products was monitored with the Applied Biosystems StepOnePlus Real-Time PCR System. Samples were normalized against murine 36b4 or human RPs13. The BioRad FGF21 primer assay (10025636) was used for FGF21 detection. Primer sequences are described in Table 27. The relative quantification of gene expression was determined using the 2-ΔΔCt method.

### Protein Purification and Gene Expression Analysis

Freshly isolated hearts and cells were homogenized in RIPA buffer containing protease inhibitors. 20-30 ug of total protein extracts were analyzed with SDS-PAGE and transferred (wet transfer) to PVDF membranes (Millipore) for Western Blotting. Membranes were blocked by 5% BSA for 1 hour and then incubated with respective antibodies (Table 28). Appropriate secondary antibodies were selected according to the host species of the primary antibodies (IRDye 680 or IRDye 800, LI-COR), and images were taken by using Li-Cor Odyssey CLx Infrared Imager (LI-COR, Lincoln, Nebraska, USA). The images were quantified with using image J.

### RNA Sequencing

Transcriptomic analysis was performed at the Single Cell Genomics Facility, Cincinnati Children's Hospital Medical Center. Briefly, the initial amplification step for all samples was done with the Ovation RNA-Seq System v2 kit (Tecan Genomics). The assay was used to amplify RNA samples to create double stranded cDNA. A total of 10 ng RNA was used per sample. The assay created between 3.2-4.3 micrograms of double stranded cDNA for each sample. The concentrations were measured using the Qubit dsDNA BR assay. The cDNA size of each sample was determined by using an Agilent HS DNA Chip.

Libraries were then created for all samples using 1 ng of cDNA. Specifically, the Illumina protocol, the Nextera XT DNA Sample Preparation Kit, was used to create DNA library templates from the double stranded cDNA. The concentrations were measured using the Qubit dsDNA HS assay. The size of the libraries for each sample was measured using the Agilent HS DNA chip. The samples were placed in a pool. The concentration of the pool was optimized to acquire at least 30 million reads per sample by using Paired-End 100 bp Reads, so for sequencing a NovaSeq SP (200 cycles) v1.5 flow cell was used. Libraries were sequenced on the Illumina NovaSeq 6000 system following the manufacturer's protocol. A quality control check on the fastq files was performed using FastQC. Upon passing basic quality metrics, the reads were trimmed to remove adapters and low-quality reads using default parameters in Trimmomatic [Version 0.33].

The trimmed reads were then mapped to a reference genome using default parameters with strandness (R for single-end and RF for paired-end) option in Hisat2 [Version 2.0.5]. In the next step, transcript/gene abundance was determined using kallisto [Version 0.43.1]. We first created a transcriptome index in kallisto using Ensembl cDNA sequences for the reference genome. This index was then used to quantify transcript abundance in raw counts and transcript per million (TPM).

DESeq2 version 1.40.2 was used to identify differentially expressed genes (DEGs) with at least 20 read counts across all samples at either 3 days (TAC vs Sham), 2 weeks (TAC vs Sham) or 8 weeks (Ctrl TAC vs Ctrl Sham, HEP-FGF21−/− TAC vs Ctrl TAC, CM-FGF21−/− TAC vs Ctrl TAC). DEGs were filtered based on FDR <0.05 and absolute log 2 fold change >0.5. Up- and downregulated gene sets from each time point were subjected to KEGG pathway enrichment analysis using “clusterProfiler” R package with a cutoff of β-values <0.5 and q-values <0.5. “Enrichplot” R package was used to visualize the upregulated and downregulated KEGG pathways. RNA-seq read counts were normalized using the “median of ratios” method for heatmap generation.

### NMR Metabolomics

The NMR based metabolomics experiments were performed at the Cincinnati Children's Hospital Medical Center NMR-Based Metabolomics Facility (RRID: SCR_022636). Each individual sample was weighed into bead tubes (2.8 mm, MO BIO, catalog #13114-50) for extraction. Modified Bligh and Dyer extraction was used to obtain polar metabolites based on the tissue weights and the average water content of mouse cardiac tissue (79%). NMR samples preparation, the data collection and data processing were performed with a Bruker Avance III HD 600 MHz spectrometer using Topspin 3.6 software (Bruker Analytik, Rheinstetten, Germany) as previously described. Total of 49 metabolites were assigned and quantified based on the chemical shifts on 1D 1H, 2D TOCSY and HSQC NMR experiments with reference spectra found in databases, Human Metabolome Database (HMDB), and Chenomx® NMR Suite profiling software (Chenomx Inc. version 8.1). The concentrations of the metabolites in polar extracts were calculated using Chenomx software based on the internal standard, TMSP (1 mM). The metabolites were normalized to the weights or the starting material. Prior to all statistical analyses, log transformation and mean centering data scaling were used for multivariate analysis. All the metabolomics data analysis were performed using MetaboAnalyst.

### Lipidome Analysis

For lipidomic analysis, the organic portion of extracted sample from two-phase extraction method was dried under nitrogen, resuspended in 200 μL solvent (acetonitrile/isopropanol/water, 35/45/20 v/v/v with 10 mM ammonium formate and 0.1% formic acid), vortexed, sonicated, centrifuged and subsequently transferred an HPLC vial. An aliquot of 20 μl of chilled methanol containing an internal standard mixture (PE(17:0/17:0), PG(18:0/16:0)-d5, PC(18:1/16:0)-d31, Spingosine(17:0), Ceramide(d18:1/17:0); SM (d18:1/17:0); Palmitic acid-d3; Cholesterol-d7; TG (17:0/17:1/17:0)-d5; DG (12:0/12:0/0:0); MG (17:0/0:0/0:0); LPS(17:0), LPE (17:1)) was added to each sample. The untargeted lipidomics analysis was conducted on a Q Exactive™ plus hybrid quadrupole-Orbitrap™ mass spectrometer interfaced with Vanquish ultra-high performance liquid chromatography (UHPLC) system (Thermo Scientific, Waltham, MA). A gradient mobile phase was used with a binary solvent system, which changed from 60% solvent A to 57% solvent A over 2 min, then to 50% solvent A at 2.1 min, then to 46% solvent A over 9.9 min, and then, after change to 30% at 12.1 min, to 1% solvent A over 5.9 min, then to 60% solvent A at 18.1 min and this was held for 2 min. The total run time was 20 min, and the flow rate was 0.4 mL/min. Solvent A consisted of acetonitrile/water (60/40) with 10 mM ammonium formate and 0.1% formic acid; solvent B consisted of isopropanol/acetonitrile (90/10) with 10 mM ammonium formate and 0.1% formic acid. The injection volume was 5 μL for both negative and positive ion mode. An Acquity CSH C18 UPLC column (2.1×100 mm, 1.7 μm, Waters, Milford, MA) was used for separation. Column temperature was set at 55° C. The ESI source was operated in the following parameters: spray voltage is 2.5 KV, capillary temperature, 350° C.; sheath gas flow rate, 35; auxiliary gas heater temperature, 325° C. Data were acquired using full MS scan (mass scan range 150-1500 m/z, AGC target 3e6, maximum IT 100 ms, resolution 140,000) and collision induced dissociation-based data dependent on MS/MS (resolution 17,500, AGC target 1e5, maximum IT 50 ms, loop count 15, top N=15, isolation window 1.0 m/z, stepped NCE 20, 40, 60). Data quality and instrument performance was monitored throughout the data acquisition using quality control (internal standards), method blanks and pooled samples. Data was processed using the Progenesis QI Analysis software (Waters, Milford, MA). Intensity values are representative of peak area. Complex lipids were identified by searching against a precursor accurate mass, retention time, in conjunction with matching tandem mass spectra library (in-house) as well as public spectral libraries including LIPID MAPS structure database (LMSD) and HMDB. Abbreviations are shown in the data supplement (Table III in the Supplement).

### Statistical Analysis

GraphPad Prism 10.2.1.395 software was employed for conducting statistical analyses. All groups with N≥6 were assessed for normal distribution with Shapiro-Wilk Test (P<0.05). Upon confirmation of normal distribution, nonpaired 2-tailed Student t test or 1- or 2-way ANOVA analysis with Tukey multiple comparisons were performed. Graphs represent means ±SEM. A P<0.05 was considered statistically significant.

### Data Availability

RNA-seq data are available in the National Center for Biotechnology Information Sequence Read Archive (SRA) Repository (BioProject PRJNA1106030). The metabolomics and lipidomics raw data are provided as supplemental materials. The corresponding author can also provide data upon request.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

### FIGURE LEGENDS

FIG. 1 is a series of images showing that hepatic and cardiac FGF21 expression is increased in humans and mice with increased afterload. Cardiac hypertrophy, cardiac function, and fibrosis-related gene expression changes during progression of cardiac hypertrophy.

A-C: Serum FGF21 (A, B) and cardiac FGF21 mRNA levels (C) in humans with left ventricular hypertrophy (A) or hypertension (B, C), compared to individuals without hypertrophy or normotensive individuals. D-F: Heart weight normalized to tibia length (D), representative short-axis M-Mode images of the left ventricle (E), and measurements of fractional shortening (F), in C57Bl/6 mice subjected control sham surgery or TAC for 3 days (no cardiac function data), 2 weeks, 4 weeks and 8 weeks post-surgery. G-K: Cardiac COL1A1, COL3A1, PSTN, β-MHC and BNP mRNA levels in the C57Bl/6 mice subjected to sham or TAC for 3 days, 2 weeks, 4 weeks, and 8 weeks (G), Plasma FGF21 (H), FGF21 mRNA in the liver (I), heart (J), and White adipose tissue (K) in C57Bl/6 mice subjected to control sham surgery or TAC for 3 days (no cardiac function data), 2 weeks, 4 weeks and 8 weeks post-surgery.

A: Healthy (no LVH), n=45; Hypertrophy, n=31. B: Normotensive, n=18; Hypertensive, n=50. C: Normotensive, n=6; Hypertensive, n=6. D: Sham, n=8; 3 days TAC, n=12; 2 weeks TAC, n=9; 4 weeks TAC, n=10; 8 weeks TAC, n=11. E: Sham, n=7; 2 weeks TAC, n=7; 4 weeks TAC, n=7; 8 weeks TAC, n=7. F: Sham, n=7; 2 weeks TAC, n=7; 4 weeks TAC, n=7; 8 weeks TAC, n=7. G: Sham, n=5-6; 3 days TAC, n=4-8; 2 weeks TAC, n=6-8; 4 weeks TAC, n=6-8; 8 weeks TAC, n=6-8. H: Sham, n=11; 3 days TAC, n=10; 2 weeks TAC, n=10; 4 weeks TAC, n=9; 8 weeks TAC, n=12. I: Sham, n=8; 3 days TAC, n=9; 2 weeks TAC, n=11; 4 weeks TAC, n=9; 8 weeks TAC, n=9. J: Sham, n=9; 3 days TAC, n=12; 2 weeks TAC, n=12; 4 weeks TAC, n=9; 8 weeks TAC, n=10. K: Sham, n=8; 3 days TAC, n=6; 2 weeks TAC, n=7; 4 weeks TAC, n=7; 8 weeks TAC, n=6. Data are presented as mean±SEM, *P<0.05, **P<0.01 ***P<0.001, ****P<0.0001; analyzed using one-way or two-way ANOVA with Tukey Multiple comparison test or Student's t-test when applicable.

FIG. 2 is a pair of images showing that changes in cardiac transcriptome, metabolome and lipidome occur as soon as 3 days post-TAC and continue throughout the progression of cardiac hypertrophy.

KEGG pathway analysis (A), and summary of metabolomic and lipidomic changes (B) depicting upregulation or downregulation in gene expression, lipids and metabolites (B) in hearts obtained from C57Bl/6 mice subjected to control sham surgery or TAC 3 days, 2 weeks, and 8 weeks post-surgery. Sham, n=5; TAC, n=5. Data are presented as mean±SEM, *P<0.05, **P<0.01, ***P<0.001, ****P<0.0001; analyzed using Student's t-test.

FIG. 3 is a series of images showing that genetic ablation of hepatocyte FGF21 lowers cardiac FGF21 expression and attenuates cardiac hypertrophy and dysfunction.

A-J: Hepatic FGF21 mRNA (A), plasma FGF21 (B), and heart weight normalized to tibia length (C), representative short-axis M-mode images of the left ventricle (D), measurements of fractional shortening (E), cardiac BNP (F) and β-MHC (G) mRNA levels, WGA staining (H upper panel, scale bar 100 m), PSR staining (H lower panel, scale bar 100 m), cardiac FGF21, pAMPKThr172, AMPK, pERK1/2, ERK, PPARα, SIRT1, pAKTSer473, and 3-actin protein levels (I) and cardiac FGF21 mRNA (J) in control (floxed or Alb-Cre+/−) and HEP-FGF21−/− mice subjected to control sham surgery or TAC 8 weeks post-surgery.

K-M: Phalloidin and DAPI staining (scale bar 100 m) (K), quantification of cardiomyocyte cell size (L), and FGF21 mRNA (M) of adult primary cardiomyocytes treated either with rm-FGF21 or control vehicle for 24 hours.

N-P: Cardiac FGFR1 mRNA levels in C57Bl/6 mice subjected to sham surgery or TAC for 3 days, 2 weeks, 4 weeks, and 8 weeks (N), in humans with hypertension or normotensive individuals (O), and in control or HEP-FGF21−/− mice subjected to sham or TAC surgery 8 weeks post-surgery (P).

A: Ctrl Sham, n=6; Ctrl TAC, n=6; HEP-FGF21−/−TAC, n=6. B: Ctrl Sham, n=6; Ctrl TAC, n=7; HEP-FGF21−/−TAC, n=7. C: Ctrl Sham, n=9; ctrl TAC, n=10, HEP-FGF21−/− TAC, n=9. D, E: Ctrl Sham, n=6; ctrl TAC, n=6, HEP-FGF21−/− TAC, n=6. F: Ctrl Sham, n=6; ctrl TAC, n=6, HEP-FGF21−/− TAC, n=6. G: Ctrl Sham, n=6; ctrl TAC, n=7, HEP-FGF21−/− TAC, n=6. H: Ctrl Sham, n=6; ctrl TAC, n=6, HEP-FGF21−/− TAC, n=6. I: Ctrl Sham, n=3-6; ctrl TAC, n=5-11, HEP-FGF21−/− TAC, n=6-10. J: Ctrl Sham, n=7; Ctrl TAC, n=7; HEP-FGF21−/−TAC, n=7. K, L: Vehicle, n=5; rmFGF21, n=6. M: Vehicle, n=6; rmFGF21, n=6. O: Normotensive, n=5; Hypertensive, n=6. P: Ctrl Sham, n=7; Ctrl TAC, n=9; HEP-FGF21−/−TAC, n=10.

Data are presented as mean±SEM, *P<0.05, **P<0.01, ***P<0.001, ****P<0.0001; analyzed using one-way ANOVA with Tukey Multiple comparison test or Student's t-test when applicable.

FIG. 4 is a series of images showing that genetic deletion of cardiomyocyte FGF21 prevents cardiac hypertrophy independent of systemic FGF21 levels.

A-L: Cardiac FGF21 mRNA (A), plasma FGF21 (B), heart weight normalized to tibia length (C) representative short-axis M-mode images of the left ventricle (D), measurements of fractional shortening (E), cardiac BNP (F) and β-MHC mRNA levels (G), WGA staining (H upper panel, scale bar 100 m), quantification of cell size area (I), PSR staining (H lower panel, scale bar 100 m), quantification of the fibrotic tissue area (J), cardiac FGF21, pAMPKThr172, AMPK, pERK1/2, ERK, PPARα, pAKTSer473, AKT, SIRT1 and β-actin protein (K), and cardiac FGFR1 mRNA in control (combined floxed and αMHC-Cre+/−) and CM-FGF21−/− mice subjected to control sham surgery or TAC 8 weeks post-surgery.

A: Ctrl Sham, n=7; Ctrl TAC, n=9; CM-FGF21−/− TAC, n=7: B: Ctrl Sham, n=6; Ctrl TAC, n=6; CM-FGF21−/−TAC, n=6; C: Ctrl Sham, n=9; Ctrl TAC, n=12; CM-FGF21−/−TAC, n=11. D-J: Ctrl Sham, n=6; Ctrl TAC, n=6; CM-FGF21−/− TAC, n=6: K: Ctrl Sham, n=3-7; Ctrl TAC, n=6-11; CM-FGF21−/− TAC, n=5-8. L: Ctrl Sham, n=6; Ctrl TAC, n=8; CM-FGF21−/−TAC, n=7.

Data are presented as mean±SEM, *P<0.05, **P<0.01, ***P<0.001, ****P<0.0001; analyzed using by one-way ANOVA with Tukey Multiple comparison test.

FIG. 5 is a series of images showing that the anti-hypertrophic effects of hepatocyte or cardiomyocyte FGF21 suppression share partial overlap in transcriptome, metabolome and lipidome profile changes; oxytocin signaling is a common activated pathway.

Hierarchical clustering (A, D), overlapping gene expression changes (B, C), and KEGG pathway analysis (E, F) depicting upregulation (A, B, F, H) or downregulation in hearts obtained from HEP-FGF21−/− or CM-FGF21−/− mice subjected to TAC and compared to control mice subjected to TAC.

G: Hypothalamic OXT mRNA levels in control mice and HEP-FGF21−/− mice subjected to sham surgery or TAC for 3 days, 2 weeks and 8 weeks post-surgery. H: Cardiac OXTR mRNA levels in C57Bl/6 mice subjected to sham or TAC for 3 days, 2 weeks, 4 weeks, and 8 weeks. I, J: Cardiac OXTR and β-actin immunoblotting (I) and quantification (J) in control mice subjected sham or TAC surgery and HEP-FGF21−/− mice after TAC 8 weeks post-surgery.

K-L: Phalloidin and DAPI staining (scale bar 100 m) (K) and quantification of cell size (L), of adult cardiomyocytes treated with rm-FGF21, combination of rm-FGF21 and increasing doses of oxytocin or control vehicle for 24 hours.

M: Summary of metabolomic and lipidomic changes depicting upregulation or downregulation in hearts obtained from HEP-FGF21−/− or CM-FGF21−/− mice subjected to TAC and compared to control mice subjected to TAC.

A-F: Ctrl TAC, n=4-5; HEP-FGF21−/− TAC, n=5; CM-FGF21−/−, n=5. G: Sham, n=7; 3 days TAC, n=6; 2 weeks TAC, n=6; 8 weeks TAC, n=6; HEP-FGF21^{-/-} 8 weeks TAC, n=5. H: Sham, n=6; 3 days TAC, n=12; 2 weeks TAC, n=11; 4 weeks TAC, n=12; 8 weeks TAC, n=6. I, J: Ctrl Sham, n=6; Ctrl TAC, n=11; HEP-FGF21−/−TAC, n=10. K, L: Vehicle, n=5; rmFGF21, n=6; rmFGF21 OXT 10 nM, n=4; rmFGF21 OXT 100 nM, n=5. M: Ctrl TAC, n=4-5; HEP-FGF21−/− TAC, n=5; CM-FGF21−/−, n=5.

Data are presented as mean±SEM, *P<0.05, **P<0.01, ***P<0.001, ****P<0.0001; analyzed using by one-way ANOVA with Tukey Multiple comparison test or Student's t-test when applicable.

FIG. 6 is a series of images showing that AAV-mediated overexpression of cardiac FGF21 negates the cardioprotective effect of hepatocyte-specific FGF21 ablation in pressure overload.

Cardiac FGF21 mRNA (A), cardiac FGF21 immunoblotting (B) and protein quantification (C), heart weight normalized to tibia length (D) representative short-axis M-mode images of the left ventricle (E) fractional shortening (F) cardiac BNP mRNA (G) cardiac β-MHC mRNA (H) WGA staining of the hearts (I, scale bar 50 m) and PSR staining of the hearts (J, scale bar 100 m) of control (floxed or Alb-Cre+/−) or HEP-FGF21−/− mice that were administered retro-orbitally either Myo-AAV91A-ctrl or Myo-AAV91A-FGF21 and subjected to either sham surgery or TAC for 8 weeks.

A: Myo-AAV91A-Ctrl Sham, n=7; Myo-AAV91A-Ctrl TAC, n=7; Myo-AAV91A-FGF21 TAC, n=8, HEP-FGF21−/− Myo-AAV91A-Ctrl TAC, n=6; HEP-FGF21−/−Myo-AAV91A-FGF21 TAC, n=6; B, C: Myo-AAV91A-Ctrl Sham, n=4; Myo-AAV91A-Ctrl TAC, n=6; Myo-AAV91A-FGF21 TAC, n=6, HEP-FGF21−/− Myo-AAV91A-Ctrl TAC, n=6; HEP-FGF21−/− Myo-AAV91A-FGF21 TAC, n=6. D: n=6; E, F: n=6; G, H: Myo-AAV91A-Ctrl Sham, n=7; Myo-AAV91A-Ctrl TAC, n=9; Myo-AAV91A-FGF21 TAC, n=9, HEP-FGF21−/− Myo-AAV91A-Ctrl TAC, n=6; HEP-FGF21−/− Myo-AAV91A-FGF21 TAC, n=6; I, J: n=6. Data are presented as mean±SEM, *P<0.05, **P<0.01, ***P<0.001, ****P<0.0001; analyzed using by one-way ANOVA with Tukey Multiple comparison test.

FIG. 7 is a series of images showing that antisense oligonucleotide mediated inhibition of FGF21 holds therapeutic potential against LVH from increased afterload.

A: Outline of the treatment of C57Bl/6 mice that were subjected to control sham surgery or TAC with control ASO-scr, ASO-FGF21 or sequential combination of both.

B-K: Hepatic (B), cardiac (C), and WAT FGF21 mRNA (D), heart weight normalized to tibia length (E), representative short-axis M-mode images of the left ventricle (F) measurements of fractional shortening (G), WGA staining (H, scale bar 50 m) and quantification of cell size (I), PSR staining (J, scale bar 100 m) and quantification of the fibrotic tissue area (K) in C57Bl/6 mice subjected to sham surgery or TAC followed by treatment with control ASO-scr for 8 weeks, ASO-FGF21 for 8 weeks or sequential treatment with ASO-scr for 2 weeks and ASO-FGF21 for 6 additional weeks.

B-D: Sham ASO Scr, n=5-6; TAC ASO Scr, n=6-7; TAC ASO FGF21 (0-8 Wk), n=5-6; TAC ASO FGF21 (2-8 Wk), n=6. E-K: Sham ASO Scr, n=6; TAC ASO Scr, n=6; TAC ASO FGF21 (0-8 Wk), n=5-6; TAC ASO FGF21 (2-8 Wk), n=6. Data are presented as mean±SEM, *P<0.05, **P<0.01 ***P<0.001, ****P<0.0001; analyzed using by one-way ANOVA with Tukey Multiple comparison test.

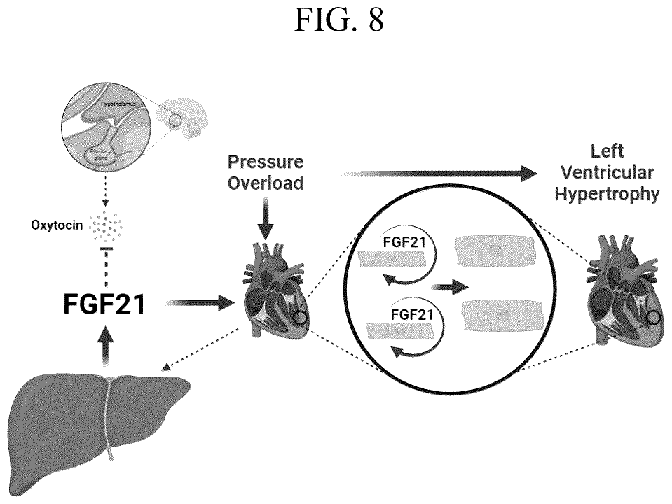

FIG. 8 is a schematic showing a graphical abstract of the invention.

FIG. 9 is a series of images showing a pressure gradient and comparison of FGF21 abundance in male and female mice; Related to FIG. 1—A. Pressure gradient in C57Bl/6 mice subjected to sham surgery or TAC for 2 weeks, 4 weeks and 8 weeks post-surgery.

B-G: Plasma FGF21 (B), and FGF21 mRNA in the liver (C), heart (D), skeletal muscle (E), brown adipose tissue (F) and white adipose tissue (G) of C57Bl/6 male or female mice subjected to control sham surgery or TAC for 3 days, 2 weeks, 4 weeks, and 8 weeks post-surgery.

A: Sham, n=7; 2 weeks TAC, n=7; 4 weeks TAC, n=7; 8 weeks TAC, n=7. B: Male sham, n=6; Female sham, n=5; Male TAC 3 days, n=6; Female TAC 3 days, n=4; Male TAC 2 weeks, n=5; Female TAC 2 weeks, n=5; Male TAC 4 weeks, n=5; Female TAC 4 weeks, n=4; Male TAC 8 weeks, n=6; Female TAC 8 weeks, n=6; C: Male sham, n=4; Female sham, n=4; Male TAC 3 days, n=5; Female TAC 3 days, n=5; Male TAC 2 weeks, n=5; Female TAC 2 weeks, n=6; Male TAC 4 weeks, n=4; Female TAC 4 weeks, n=5; Male TAC 8 weeks, n=6; Female TAC 8 weeks, n=4; D: Male sham, n=5; Female sham, n=4; Male TAC 3 days, n=5; Female TAC 3 days, n=5; Male TAC 2 weeks, n=5; Female TAC 2 weeks, n=6; Male TAC 4 weeks, n=4; Female TAC 4 weeks, n=5; Male TAC 8 weeks, n=6; Female TAC 8 weeks, n=4; E: Sham, n=9; 3 days TAC, n=7; 2 weeks TAC, n=6; 4 weeks TAC, n=6; 8 weeks TAC, n=6. F: Sham, n=5; 3 days TAC, n=8; 2 weeks TAC, n=8; 4 weeks TAC, n=8; 8 weeks TAC, n=8. G: Male sham, n=5; Female sham, n=3; Male TAC 3 days, n=3; Female TAC 3 days, n=3; Male TAC 2 weeks, n=4; Female TAC 2 weeks, n=3; Male TAC 4 weeks, n=4; Female TAC 4 weeks, n=3; Male TAC 8 weeks, n=3; Female TAC 8 weeks, n=3.

Data are presented as mean±SEM, *P<0.05, **P<0.01, ***P<0.001, ****P<0.0001; analyzed using one-way or two-way ANOVA with Tukey Multiple comparison test.

FIG. 10 is a series of images showing a KEGG pathway analysis of differentially regulated genes at 3 days, 2 weeks, and 8 weeks post-TAC; Related to FIG. 2. KEGG pathway analysis of upregulated genes (A-C) or downregulated genes (D-F) in cardiac transcriptome of C57Bl/6 mice subjected to sham or TAC for 3 days (A), 2 weeks (B), and 8 weeks (C). A-F: n=5. Data are presented as mean±SEM, *P<0.05, **P<0.01 ***P<0.001, ****P<0.0001; analyzed using student t-test.

FIG. 1I is a series of images showing that hemodynamic congestion in the liver caused by pIVCL or TAC induces the expression of hepatic FGF21 and mechano-sensor-related genes; Related to FIG. 1—Hepatic FGF21 (A), LRRC8A (B, C), LRRC8B (D, E), and PIEZO1 (F, G) mRNA levels in C57Bl/6 mice subjected to pIVCL12 weeks post-surgery (A, C, E, G) or TAC for 3 days, 2 weeks, 4 weeks, and 8 weeks post-surgery (B, D, F) and control sham surgery.

A, C, E, G: Sham, n=3; pIVCL, n=5. B: Sham, n=7; 3 days TAC, n=11; 2 weeks TAC, n=9; 4 weeks TAC, n=8; 8 weeks TAC, n=8. D: Sham, n=7; 3 days TAC, n=12; 2 weeks TAC, n=9; 4 weeks TAC, n=8; 8 weeks TAC, n=6. F: Sham, n=7; 3 days TAC, n=9; 2 weeks TAC, n=10; 4 weeks TAC, n=11; 8 weeks TAC, n=10.

Data are presented as mean±SEM, *P<0.05, **P<0.01, ***P<0.001, ****P<0.0001; analyzed using by one-way ANOVA with Tukey Multiple comparison test or Student's t-test when applicable.

FIG. 12 is a series of images showing that FGF21^{f1/f1}, Alb-Cre^{+/-} and αMHC-Cre^{+/-} mice respond similarly to pressure overload; Related to FIG. 3—A-C: FGF21 mRNA in the liver (A), heart (B) and white adipose tissue (C) of FGF21^{f1/f1}, Alb-Cre^{+/-} and HEP-FGF21^{-/-} mice. D-G: Heart weight normalized to tibia length (D) fractional shortening (E), cardiac β-MHC mRNA (F) and cardiac COL3A1 mRNA (G) in FGF21^{f1/f1}, Alb-Cre^{+/-} and α-MHC-Cre^{+/-} mice subjected to sham or TAC surgery for 8 weeks.

A: FGF21^{f1/f1}, n=14; Alb-Cre^{+/-}, n=6; HEP-FGF21−/−, n=6. B: FGF21¹¹, n=7; Alb-Cre^{+/-}, n=6; HEP-FGF21^{-/-}, n=7. C: n=6. D-G: n=6. Data are presented as mean±SEM, *P<0.05, **P<0.01, ***P<0.001, ****P<0.0001; analyzed using by one-way ANOVA with Tukey Multiple comparison test.

FIG. 13 is a series of images showing that both male and female mice with hepatocyte-specific FGF21 deletion exhibit similar responses to pressure overload-induced FGF21 expression, cardiac hypertrophy and fibrosis; Related to FIG. 3—A. Pressure gradient in control (mixed FGF21^{f1/f1} and Alb-Cre^{+/-}) mice and HEP-FGF21^{-/-} subjected to sham or TAC surgery 8 weeks post-surgery. B-E: Hepatic FGF21 mRNA (B), plasma FGF21 (C), heart weight normalized to tibia length (D), fractional shortening (E) in male and female control mice (FGF21^{f1/f1} and Alb-Cre^{+/-}) and HEP-FGF21^{-/-} mice subjected to sham or TAC surgery 8 weeks post-surgery.

F, G: Quantification of cell size area (F), and fibrotic tissue area (G) in control (mixed FGF21^{f1/f1} and Alb-Cre^{+/-}) mice and HEP-FGF21^{-/-} subjected to sham or TAC surgery 8 weeks post-surgery. (blue color indicates male and red color indicates female mice).

A, B, E, F, G: n=6; C: Male Ctrl Sham, n=3; Female Ctrl Sham, n=3; Male Ctrl TAC, n=4, Female Ctrl TAC, n=3; Male HEP-FGF21^{-/-} TAC, n=4; Female HEP-FGF21^{-/-} TAC, n=3. D: Male Ctrl Sham, n=4; Female Ctrl Sham, n=5; Male Ctrl TAC, n=6, Female Ctrl TAC, n=4; Male HEP-FGF21^{-/-} TAC, n=5; Female HEP-FGF21^{-/-} TAC, n=4.

Data are presented as mean±SEM, *P<0.05, **P<0.01, ***P<0.001, ****P<0.0001; analyzed using by one-way or two-way ANOVA with Tukey Multiple comparison test.

FIG. 14 is a series of images showing that hepatocyte-specific Fgf21 ablation attenuates cardiac fibrosis- and inflammation-related gene expression, and pro-hypertrophic cellular signaling pathways; Related to FIG. 3. Cardiac COL1A (A), COL3A1 (B), PSTN (C), IL-1α (D), IL-1β (E), and TNFα (F) mRNA, plasma IL-1α(G), IL-1β (H), and TNFα (I), cardiac IL-10 mRNA (J), plasma IL-10 (K), quantification of cardiac pAMPKThr¹⁷²/AMPK/β-actin (L), pERK1/2/ERK/0-actin (M), FGF21/β-actin protein (N), and FGF21 mRNA levels in skeletal muscle (O), brown adipose tissue (P), and white adipose tissue (Q) in control mice and HEP-FGF21^{-/-} mice 8 weeks post-TAC or control sham surgery.

A-C: Ctrl Sham, n=6; Ctrl TAC, n=10; HEP-FGF21−/− TAC, n=8. D-F: Ctrl sham, n=6; Ctrl TAC, n=6-9; HEP-FGF21−/− TAC, n=6-8. G-I: Ctrl Sham, n=4-6; Ctrl TAC, n=5-6; HEP-FGF21^{-/-} TAC, n=5-6. J-K: Ctrl Sham, n=3; Ctrl TAC, n=4-6; HEP-FGF21^{-/-} TAC, n=4-5. L-N: Ctrl Sham, n=3; Ctrl TAC, n=5; HEP-FGF21^{-/-}, TAC, n=6. O-Q: n=6. Data are presented as mean±SEM, *P<0.05, **P<0.01 ***P<0.001, ****P<0.0001; analyzed using one-way ANOVA with Tukey Multiple comparison test.

FIG. 15 is a series of images showing that treatment of AC16 cells (human cardiomyocyte cell line) with recombinant FGF21 activates endogenous FGF21 expression and ERK signaling. Cardiac expression of FGF receptors and co-receptor β-klotho in mice with TAC and AC16 cells treated with recombinant FGF21; Related to FIG. 3—A-G. Phalloidin and DAPI staining (A), quantification of myocyte cell size (B), FGF21 mRNA (C), and protein (Western Blotting) (D) and protein quantification (E), pERK1/2, ERK, β-actin immunoblotting (F) and quantification (G) of AC16 cells treated either with rh-FGF21 or control vehicle for 24 hours.

H-I: Cardiac FGFR4 (H) and R KLOTHO (I) mRNA levels in C57Bl/6 mice subjected to control sham surgery or TAC for 3 days, 2 weeks, 4 weeks, and 8 weeks post-surgery.

J: Cardiac FGFR4 mRNA levels humans with hypertension compared to normotensive individuals. K, L: FGFR1 and FGFR4 mRNA levels of AC16 cells treated with rh-FGF21 or control vehicle for 24 hours.

A-G: n=6. H: Sham, n=9; 3 days TAC, n=12; 2 weeks TAC, n=7; 4 weeks TAC, n=7; 8 weeks TAC, n=7. I: Sham, n=8; 3 days TAC, n=9; 2 weeks TAC, n=10; 4 weeks TAC, n=10; 8 weeks TAC, n=10. J: n=6. K, L: control vehicle, n=4; rh-FGF21, n=6.

Data are presented as mean±SEM, *P<0.05, **P<0.01, ***P<0.001, ****P<0.0001; analyzed using by one-way ANOVA with Tukey Multiple comparison test or Student's t-test when applicable.

FIG. 16 is a series of images showing that hepatocyte-specific FGF21 ablation reduced pressure overload-induced cardiac PPARα, Sirtuin1 and pAKT/tAKT protein abundance; Related to FIG. 4. Cardiac PPARα (A), Sirtuin1 (B) and pAKT normalized to total AKT and β-actin immunoblotting quantification (C) in control mice and HEP-FGF21^{-/-} mice 8 weeks post-TAC or control sham surgery

A, C: Ctrl Sham, n=3; Ctrl TAC, n=5; HEP-FGF21−/− TAC, n=6. B: Ctrl Sham, n=6; Ctrl TAC, n=11; HEP-FGF21−/− TAC, n=10. Data are presented as mean±SEM, *P<0.05, **P<0.01, ***P<0.001, ****P<0.0001; analyzed using by one-way ANOVA with Tukey Multiple comparison test.

FIG. 17 is a series of images showing that FGF21 expression in cardiomyocytes, liver and white adipose tissue of mice with cardiomyocyte-specific FGF21 ablation, both male and female mice, exhibit similar FGF21 expression, as well as cardiac hypertrophy- and fibrosis-related gene expression in response to pressure overload; Related to FIG. 4.

A-C: FGF21 mRNA levels in cardiomyocytes (A), liver (B) and white adipose tissue (C) in FGF21^{f1/f1}, α-MHC-Cre^{+/-} and CM-FGF21^{-/-} mice. D: Pressure gradient in control (FGF21^{f1/f1} and α-MHC-Cre^{+/-}) and CM-FGF21^{-/-} mice subjected to sham or TAC surgery 8 weeks post-surgery. E-I: Cardiac FGF21 mRNA (E), hepatic FGF21 mRNA (F), plasma FGF21 (G), heart weight normalized to tibia length (H), and fractional shortening (I) in male and female control and CM-FGF21^{-/-} mice subjected to sham or TAC surgery 8 weeks post-surgery. J-L: Cardiac COL1A1 (J), COL3A1 (K), and PSTN (L), in Ctrl and CM-FGF21^{-/-} mice subjected to sham or TAC surgery 8 weeks post-surgery.

A: FGF21^{f1/f1}, n=15; α-MHC-Cre+/−, n=12; CM-FGF21^{-/-}, n=7; B: FGF21^{f1/f1}, n=10; α-MHC-Cre+/−, n=8; CM-FGF21−/−, n=7. C, D: n=6. E: Male Ctrl Sham, n=3; Female Ctrl Sham, n=4; Male Ctrl TAC, n=4, Female Ctrl TAC, n=5; Male CM-FGF21^{-/-}TAC, n=4; Female CM-FGF21^{-/-} TAC, n=3. F, G, I: n=3. H: Male Ctrl Sham, n=4; Female Ctrl Sham, n=5; Male Ctrl TAC, n=6, Female Ctrl TAC, n=5; Male CM-FGF21^{-/-} TAC, n=5; Female CM-FGF21^{-/-} TAC, n=6. J, K: Ctrl Sham, n=6; Ctrl TAC, n=7; CM-FGF21 TAC, n=7. L: Ctrl Sham, n=6; Ctrl TAC, n=9; CM-FGF21^{-/-}TAC, n=9:

Data are presented as mean±SEM, *P<0.05, **P<0.01 ***P<0.001, ****P<0.0001; analyzed using by one-way ANOVA with Tukey Multiple comparison test or Student's t-test when applicable.

FIG. 18 is a series of images showing that cardiomyocyte-specific FGF21 ablation attenuates pressure overload-induced cardiac and systemic inflammation as well as pro-hypertrophic cellular signaling pathways; Related to FIG. 4.

Cardiac IL-1α(A), IL-1β (B), and TNFα (C) mRNA, plasma IL-1α(D), IL-1β (E) TNFα (F), cardiac IL-1β mRNA (G) and plasma IL-1β (H), cardiac FGF21 (I), pAMPK^{Thr172} normalized to total AMPK and β-actin (J), pERK1/2 normalized to total ERK and β-actin (K), PPARα normalized to β-actin (L), pAKT normalized to total AKT (M), Sirtuin1 normalized to β-actin (N), and cardiac β-klotho (O) protein levels in control and CM-FGF21−/− mice subjected to control sham or TAC surgery 8 weeks post-surgery.

A: Ctrl sham, n=5; Ctrl TAC, n=7; CM-FGF21^{-/-}TAC, n=5. B: Ctrl sham, n=6; Ctrl TAC, n=9; CM-FGF21 TAC, n=9: C: Ctrl sham, n=6; Ctrl TAC, n=6; CM-FGF21^{-/-}TAC, n=8. D: Ctrl Sham, n=6; Ctrl TAC, n=6; CM-FGF21^{-/-}TAC, n=8. E: Ctrl sham, n=4; Ctrl TAC, n=5; CM-FGF21 TAC, n=7: F: Ctrl Sham, n=5; Ctrl TAC, n=7; CM-FGF21 TAC, n=7. G: Ctrl sham, n=3; Ctrl TAC, n=5; CM-FGF21^{-/-}TAC, n=4; H: Ctrl sham, n=3; Ctrl TAC, n=5; CM-FGF21^{-/-}TAC, n=7. I, L, M, N: Ctrl sham, n=3; Ctrl TAC, n=6; CM-FGF21^{-/-}TAC, n=5. J, K: Ctrl sham, n=7; Ctrl TAC, n=11; CM-FGF21^{-/-}TAC, n=8. O: Ctrl sham, n=7; Ctrl TAC, n=7; CM-FGF21^{-/-}TAC, n=7.

Data are presented as mean±SEM, *P<0.05, **P<0.01, ***P<0.001, ****P<0.0001; analyzed using by one-way ANOVA with Tukey Multiple comparison test.

FIG. 19 is a series of images showing that KEGG analysis of transcriptome for downregulated pathways in HEP-FGF21−/− and CM-FGF21−/− mice 8 weeks post-TAC; Related to FIG. 5. Top-downregulated pathways (A, C) and hypertrophy-related pathway components (B, D) in HEP-FGF21^{-/-} mice (A, B) or CM-FGF21^{-/-} mice (C, D) compared to control mice 8 weeks post-TAC.

A-D: Ctrl TAC, n=4; CM-FGF21^{-/-}TAC, n=5, CM-FGF21^{-/-}TAC, n=5. Data are presented as mean±SEM, *P<0.05, **P<0.01 ***P<0.001, ****P<0.0001; analyzed using student t-test.

FIG. 20 is a series of images showing that KEGG analysis of transcriptome for upregulated pathways in HEP-FGF21−/− and CM-FGF21−/− mice 8 weeks post-TAC; Related to FIG. 5. Top-upregulated pathways (A, C) and hypertrophy-related pathway components (B, D) in HEP-FGF21^{-/-} mice (A, B) or CM-FGF21−/− mice (C, D) compared to control mice 8 weeks post-TAC.

A-D: Ctrl TAC, n=4; CM-FGF21^{-/-}TAC, n=5, CM-FGF21^{-/-}TAC, n=5. Data are presented as mean±SEM, *P<0.05, **P<0.01 ***P<0.001, ****P<0.0001; analyzed using student t-test.

FIG. 21 is a series of images showing that expression of cardiac oxytocin during cardiac hypertrophic growth in mouse and human hearts; Related to FIG. 5. Cardiac OXT mRNA levels in mice subjected to control sham surgery or TAC for 3 days, 2 weeks, 4 weeks, and 8 weeks post-surgery (A) and in humans with hypertension compared to normotensive individuals (B).

A: Sham, n=6; 3 days TAC, n=11; 2 weeks TAC, n=11; 4 weeks TAC, n=6; 8 weeks TAC, n=6. B: Normotensive, n=6; Hypertensive, n=6.

Data are presented as mean±SEM, *P<0.05, **P<0.01 ***P<0.001, ****P<0.0001; analyzed using by one-way ANOVA with Tukey Multiple comparison test or Student's t-test when applicable.

FIG. 22 is a series of images showing that AAV-mediated cardiomyocyte-specific overexpression of FGF21 negates the protective effect of hepatocyte FGF21 ablation against cardiac hypertrophy; Related to FIG. 6.

Quantification of cellular area based on WGA staining (A), quantification of myocardial fibrotic area based on PSR staining (B), cardiac COL3A1 mRNA (C) and cardiac PSTN mRNA (D) in control or HEP-FGF21−/− mice that were administered either control MyoAAV1 or MyoAAV1-FGF21 and were subjected to either sham or TAC surgery for 8 weeks.

A, B: n=6. C: Myo AAV-Ctrl Sham, n=7; Myo AAV-Ctrl TAC, n=8; Myo AAV-FGF21 TAC, n=8, HEP-FGF21^{-/-}Myo AAV-Ctrl TAC, n=6; HEP-FGF21^{-/-}Myo AAV-FGF21 TAC, n=6. D: Myo AAV-Ctrl Sham, n=7; Myo AAV-Ctrl TAC, n=9; Myo AAV-FGF21 TAC, n=9, HEP-FGF21^{-/-}Myo AAV-Ctrl TAC, n=6; HEP-FGF21^{-/-}Myo AAV-FGF21 TAC, n=6. Data are presented as mean±SEM, *P<0.05, **P<0.01 ***P<0.001, ****P<0.0001; analyzed using by one-way ANOVA with Tukey Multiple comparison test.

FIG. 23 is a series of images showing selection and validation of ASO-FGF21, Related to FIG. 7—A, B: Screening of ten ASO-FGF21 (250 nM or 500 nM) for effective suppression of FGF21 mRNA normalized to 36B4 in HL-1 cells (A) and validation of the selected ASO-FGF21 in HL-1 cells (B). C-E: Cardiac (C) and hepatic (D) FGF21 mRNA levels and plasma FGF21 (E) in C57Bl/6 mice treated with either FGF21-ASO or scrambled ASO (ASO-scr) weekly for 4 weeks.

A (selection): n=1. B (validation): n=6. C: Ctrl ASO, n=6; FGF21-ASO, n=5. D: n=6. E: Ctrl ASO, n=6; FGF21-ASO, n=7.

Data are presented as mean±SEM, **P<0.01 ***P<0.001; analyzed using student t-test.

FIG. 24 is a series of images showing that ASO-FGF21 reduced gene expression related to cardiac fibrosis, inflammation and heart failure in mice with TAC, Related to FIG. 7.

A-H: Cardiac COL1A1 (A) COL3A1 (B) PSTN (C) IL-1α(D), IL-1β (E), TNFα (F), BNP (G) and β-MHC (H) mRNA in C57Bl/6 mice subjected to control sham surgery or TAC and treated with either ASO-FGF21 or ASO-scr weekly for 8 weeks.

A-H: Sham ASO Scr, n=6; TAC ASO Scr, n=8; TAC ASO FGF21 (0-8 Wk), n=6; TAC ASO FGF21 (2-8 Wk), n=6. Data are presented as mean±SEM, *P<0.05, **P<0.01 ***P<0.001, ****P<0.0001; analyzed using by one-way ANOVA with Tukey Multiple comparison test.

### Tables

TABLE 1
Demographic and clinical data of human participants
with left ventricular hypertrophy or without that
were subjected to assessment of serum FGF21

| | No LVH | LVH | |
|---|---|---|---|
| | (n = 28) | (n = 27) | p |
| Sex (male/female) | 9/19 | 7/20 | |
| Age (years) | 70 ± 9 | 70 ± 7 | 0.9 |
| Type 2 Diabetes | 3 | 8 | |
| Body Mass Index (Kg/m2) | 28.5 ± 4.3 | 31.2 ± 3.7 | 0.01 |
| Systolic Blood Pressure (mmHg) | 134 ± 12 | 134 ± 11 | 0.82 |
| Diastolic Blood Pressure (mmHg) | 76 ± 10 | 77 ± 8 | 0.68 |
| Heart Rate (bpm) | 73 ± 8 | 76 ± 9 | 0.18 |
| Glucose (mg/dL) | 92 ± 7 | 99 ± 25 | 0.18 |
| Creatinine (mg/dL) | 1.1 ± 0.2 | 1.2 ± 0.3 | 0.8 |
| Hemoglobin (mg/dL) | 13.5 ± 1.3 | 13.1 ± 0.4 | 0.39 |
| Total Cholesterol (mg/dL) | 222 ± 35 | 224 ± 47 | 0.82 |
| Uric acid (mg/dL) | 6.9 ± 1.6 | 6.8 ± 1.2 | 0.76 |
| Ejection fraction (%) | 60 ± 4 | 61 ± 5 | 0.4 |
| Left ventricular mass index (g/m²) | 82.4 ± 14 | 112.2 ± 15.6 | <0.01 |
| Left atrial diameter (mm) | 37.7 ± 3.7 | 45 ± 3 | <0.001 |
| E/e′ | 7.8 ± 2.6 | 11 ± 3.5 | <0.001 |

TABLE 2
Demographic and clinical data of human normotensive or hypertensive
participants that were subjected to assessment of serum FGF21

| | Normotensive | Hypertensive | |
|---|---|---|---|
| | (n = 21) | (n = 55) | p |
| Sex (male/female) | 5/16 | 16/39 | |
| Age (years) | 74 ± 5 | 71 ± 8 | 0.06 |
| Type 2 Diabetes | 6 | 11 | |
| Body Mass Index (Kg/m2) | 30.9 ± 5.3 | 29.8 ± 4.2 | 0.36 |
| Systolic Blood Pressure (mmHg) | 133 ± 14 | 134 ± 12 | 0.72 |
| Diastolic Blood Pressure | 76 ± 6 | 76 ± 8 | 0.9 |
| (mmHg) | | | |
| Heart Rate (bpm) | 76 ± 10 | 74 ± 9 | 0.35 |
| Glucose (mg/dL) | 103 ± 22 | 95 ± 18 | 0.12 |
| Creatinine (mg/dL) | 1.1 ± 0.2 | 1.1 ± 0.3 | 0.9 |
| Hemoglobin (mg/dL) | 14.1 ± 0.9 | 13.4 ± 1.3 | 0.052 |
| Total Cholesterol (mg/dL) | 225 ± 36 | 223 ± 41 | 0.86 |
| Uric acid (mg/dL) | 6.8 ± 1.2 | 6.9 ± 1.6 | 0.76 |
| Ejection fraction (%) | 60 ± 5 | 61 ± 4 | 0.7 |
| Left ventricular mass index | 86 ± 20 | 97 ± 21 | 0.044 |
| (g/m²) | | | |
| Left atrial diameter (mm) | 37.6 ± 3.5 | 40.5 ± 5 | 0.02 |
| E/e′ | 8 ± 0.5 | 9.9 ± 2.3 | <0.001 |

TABLE 3
Demographic and clinical data of human normotensive
or hypertensive individuals (provided by the National
Disease Research Interchange) that were subjected
to assessment of cardiac FGF21 mRNA levels.

| | Normotensive (n = 6) | | Hypertensive (n = 6) | |
|---|---|---|---|---|
| Age | 67.1 ± 19.4 | | 75 ± 14.53 | |
| Sex (male/female) | 2/4 | | 2/4 | |
| Caucasian Race | 100% | | 100% | |
| Hypertension | 0 | | 6 | |
| Obesity | 1 | (16.7%) | 2 | (33.3%) |
| Heart Disease | 0 | | 3 | (50%) |
| Hyperlipidemia | 1 | (16.7%) | 3 | (50%) |
| Kidney Disease | 1 | (16.7%) | 3 | (50%) |
| Hepatic Disease | 0 | | 0 | |
| Respiratory Disease | 2 | (33.3%) | 1 | (16.7%) |
| Tissue Recovery | | | | |
| Location | | | | |

| | | |
|---|---|---|
| Left Ventricle | 4 | 1 |
| Right Ventricle | 1 | 1 |
| Apex | 1 | 4 |

TABLE 4
2D-Echocardiography for Sham, 2 weeks, 4 weeks and 8 weeks post TAC

| | Sham | | 2 Weeks post-TAC | | 4 Weeks post-TAC | | 8 Weeks post-TAC | |
|---|---|---|---|---|---|---|---|---|
| M-Mode | Mean | SEM | Mean | SEM | Mean | SEM | Mean | SEM |
| LVD(s) | 1.929 | 0.0693 | 2.397* | 0.059 | 2.647*** | 0.083 | 3.238**** | 0.153 |
| LVD(d) | 3.40 | 0.072 | 3.594 | 0.107 | 3.671 | 0.085 | 4.067* | 0.138 |
| LV Vol(s) | 11.808 | 1.012 | 20.306 | 1.311 | 26.011** | 1.858 | 43.061**** | 4.946 |
| LV Vol(d) | 47.631 | 2.384 | 54.617 | 3.903 | 57.305 | 3.175 | 73.507** | 5.847 |
| Stroke | 35.823 | 1.661 | 34.311 | 2.819 | 31.294 | 2.116 | 30.446 | 1.53 |
| Volume | | | | | | | | |
| EF (%) | 75.369 | 1.386 | 62.579*** | 1.422 | 54.657**** | 2.350 | 42.157**** | 2.299 |
| FS (%) | 43.326 | 1.241 | 33.212*** | 1.055 | 27.902**** | 1.530 | 20.534**** | 1.267 |
| Cardiac | 18.625 | 0.979 | 15.284 | 1.714 | 15.734 | 1.360 | 15.183 | 0.676 |
| Output | | | | | | | | |
| LV Mass | 102.792 | 12.946 | 157.847 | 24.079 | 173.953 | 42.342 | 190.048 | 37.898 |
| LVAW(s) | 1.499 | 0.237 | 1.719 | 0.184 | 1.658 | 0.128 | 1.609 | 0.087 |
| LVAW(d) | 1.038 | 0.131 | 1.254 | 0.168 | 1.222 | 0.098 | 1.265 | 0.073 |
| LVPW(s) | 1.366 | 0.139 | 1.633 | 0.147 | 1.667 | 0.392 | 1.565 | 0.375 |
| LVPW(d) | 0.976 | 0.146 | 1.2642 | 0.140 | 1.347 | 0.320 | 1.247 | 0.306 |
| Heart Rate | 519.79 | 14.989 | 490.841 | 11.091 | 500.07 | 13.001 | 500.056 | 12.876 |

Mean and SEM for echocardiography parameters measured using M-Mode short axis analysis at 2 weeks, 4 weeks, and 8 weeks post-TAC or control sham surgery. Sham n=6; 2 weeks post-TAC, n=6; 4 Weeks post-TAC, n=6; 8 Weeks post-TAC, n=6. *p<0.05, **p<0.01***p<0.001, ****p<0.0001 vs Sham by one-way ANOVA with Tukey Multiple comparison test.

TABLE 5
2D-Echocardiography for Sham, 2 weeks, 4 weeks and 8 weeks post TAC

| | Sham | | 2 Weeks post-TAC | | 4 Weeks post-TAC | | 8 Weeks post-TAC | |
|---|---|---|---|---|---|---|---|---|
| B-Mode | Mean | SEM | Mean | SEM | Mean | SEM | Mean | SEM |
| EDV (μl) | 23.688 | 4.826 | 20.775 | 1.305 | 29.98 | 4.394 | 49.170 | 8.416 |
| ESV (μl) | 6.836 | 1.883 | 9.655 | 0.939 | 16.105 | 2.631 | 35.198*** | 7.403 |
| SV (μl) | 16.848 | 3.036 | 11.038 | 1.147 | 13.871 | 2.003 | 13.965 | 2.112 |
| FS (%) | 40.39 | 3.072 | 30.013* | 1.990 | 24.112*** | 2.724 | 14.887**** | 1.897 |
| CO (ml/min) | 7.845 | 1.403 | 5.423 | 0.509 | 6.761 | 1.032 | 7.112 | 1.029 |
| GLS (Peak %) | 21.398 | 2.078 | 16.268 | 2.736 | 13.470* | 0.787 | 8.058*** | 1.423 |
| EF (%) | 72.208 | 2.488 | 52.531** | 4.219 | 45.908*** | 2.265 | 29.995**** | 5.261 |
| Heart Rate | 483.833 | 12.499 | 499.666 | 18.568 | 502 | 14.301 | 505.333 | 6.205 |

Mean and SEM for echocardiography parameters measured using B-Mode long axis analysis at 2 weeks, 4 weeks, and 8 weeks post-sham or TAC surgery. Sham n=6; 2 weeks post-TAC, n=12; 4 Weeks post-TAC, n=10; 8 Weeks post-TAC, n=6. *p<0.05, **p<0.01***p<0.001, ****p<0.0001 vs Sham by one-way ANOVA with Tukey Multiple comparison test.

TABLE 6
Cardiac metabolomic analysis 3 days post-TAC

TAC vs Sham (3 days)

| Metabolites | FC | P value |
|---|---|---|
| 3-Hydroxybutyrate | 1.6705 | 0.047257 |
| AMP | 0.56998 | 0.00014301 |
| Choline | 0.58675 | 0.0011884 |
| Citrate | 0.61812 | 0.0058538 |
| Creatine | 0.62519 | 0.00034705 |
| Glucose | 0.069552 | 0.00084009 |
| Glutamate | 0.66793 | 0.001545 |
| Glutamine | 0.79261 | 0.035056 |
| Histidine | 0.69823 | 0.00035312 |
| IMP | 0.16307 | 0.01537 |
| Methionine | 1.4763 | 0.015195 |
| myo-Inositol | 0.52788 | 0.00014287 |
| N,N-Dimethylglycine | 0.67744 | 0.0011502 |
| NAD⁺ | 0.81003 | 0.044427 |
| Niacinamide | 0.50534 | 2.58E−05 |
| O-Phosphocholine | 0.5593 | 2.87E−05 |
| Phenylalanine | 0.66879 | 0.029403 |
| Pyruvate | 0.71731 | 0.0060477 |
| sn-Glycero-3-phosphocholine | 0.62057 | 0.0019289 |
| Taurine | 0.75689 | 0.00015667 |

Cardiac metabolites that are altered significantly (p<0.05) in mice 3 days post-TAC compared to control mice 3 days post-sham surgery. Statistical analysis was performed by student t-test. Sham, n=5; 3 days TAC, n=5.

TABLE 7
Cardiac metabolomic analysis 2 weeks post-TAC

TAC vs Sham (2 weeks)

| Metabolites | FC | P value |
|---|---|---|
| Acetate | 0.60203 | 0.011017 |
| Asparagine | 2.0574 | 0.048614 |
| Aspartate | 1.8811 | 0.0066161 |
| Choline | 0.64242 | 0.014666 |
| Formate | 0.71623 | 0.047433 |
| Glucose | 0.07298 | 0.00015716 |
| IMP | 0.25132 | 0.002328 |
| Methionine | 1.5259 | 0.031414 |
| myo-Inositol | 0.68681 | 0.020675 |
| Niacinamide | 0.55958 | 0.0097421 |
| O-Acetylcarnitine | 1.4233 | 0.032855 |
| Serine | 1.58 | 0.012426 |
| Succinate | 1.5429 | 0.013283 |
| Threonine | 1.4503 | 0.022345 |

Cardiac metabolites that are altered significantly (p<0.05) in mice 2 weeks post-TAC compared to control mice 2 weeks post-sham surgery. Statistical analysis was performed by student t-test. Sham, n=5; 2 weeks TAC, n=5.

TABLE 8
Cardiac metabolomic analysis 8 weeks post-TAC

TAC vs Sham (8 Weeks)

| Metabolites | FC | P value |
|---|---|---|
| Leucine | 2.4371 | 0.0013177 |
| Valine | 2.1805 | 0.0021812 |
| Methionine | 1.9378 | 0.04122 |
| Isoleucine | 1.8273 | 0.0078857 |
| O-Acetylcarnitine | 1.7272 | 0.048976 |
| Serine | 1.692 | 0.0034017 |
| Asparagine | 1.5998 | 0.0039785 |
| Alanine | 1.43 | 0.040849 |
| Glutathione | 1.3624 | 0.016269 |
| Glycine | 1.2402 | 0.023012 |
| Threonine | 1.1831 | 0.026192 |
| Histidine | 0.83277 | 0.040379 |
| NAD⁺ | 0.8031 | 0.0023879 |
| ATP | 0.71309 | 0.024638 |
| Acetate | 0.69371 | 0.0038916 |
| O-Phosphoethanolamine | 0.69025 | 0.016205 |
| IMP | 0.56106 | 0.0045738 |

Cardiac metabolites that are altered significantly (p<0.05) in mice 8 weeks post-TAC compared to control mice 8 weeks post-sham surgery. Statistical analysis was performed by student t-test. Sham, n=5; 8 weeks TAC, n=5.

TABLE 9
Cardiac lipidomic analysis 3 days post-TAC

| | TAC vs Sham (3 Days) | | |
|---|---|---|---|
| Lipid Species | Fold Change | log2(FC) | P Value |
| CL (16:0_18:0_18:1_18:2) | 2.0222 | 1.0159 | 0.012925 |
| CL (16:0_18:1_16:0_18:1) | 1.7405 | 0.79947 | 0.040755 |
| CL (18:2_18:1_18:1_16:0) | 1.5884 | 0.66757 | 0.041737 |
| CL (18:2_18:2_18:2_0:0) | 0.43331 | −1.2065 | 0.024385 |
| DG (18:2/18:3) | 0.41387 | −1.2728 | 0.0019338 |
| DG 34:2 | 0.57747 | −0.79218 | 0.012723 |
| DG 36:4 | 0.40626 | −1.2995 | 0.0019133 |
| DG 38:6b | 0.4592 | −1.1228 | 0.021636 |
| DG 38:7 | 0.14598 | −2.7762 | 0.0015603 |
| DG 40:7b | 0.25489 | −1.9721 | 3.89E−05 |
| LPC 18:1 | 1.9377 | 0.95437 | 0.0029779 |
| LPE O-18:1 | 0.49171 | −1.0241 | 0.032652 |
| PC 32:1 | 1.8338 | 0.8748 | 0.002397 |
| PC 32:2 | 1.664 | 0.73467 | 0.030361 |
| PC 33:2 | 2.2786 | 1.1881 | 0.0036318 |
| PC 34:3 | 1.504 | 0.58881 | 0.015099 |
| PC 40:5b | 0.63928 | −0.64548 | 0.003195 |
| PC 40:7 | 0.60357 | −0.7284 | 0.01068 |
| PC 40:9 | 0.51697 | −0.95185 | 0.0011824 |
| PC 42:11 | 0.371 | −1.4305 | 0.00025732 |
| PC 44:11b | 0.51961 | −0.94451 | 0.0096912 |
| PC O-34:3b | 0.46518 | −1.1041 | 0.0053441 |
| PE (16:0/22:6 (OH))c | 0.36541 | −1.4524 | 0.00066329 |
| PE (18:0/22:6)a | 0.48547 | −1.0426 | 0.0097378 |
| PE (18:1/22:8) | 0.48238 | −1.0518 | 0.0025949 |
| PE (18:1/24:0) | 1.9514 | 0.96454 | 0.0072352 |
| PE (18:4/22:6) | 0.2531 | −1.9822 | 0.0018038 |
| PE (P-18:0/16:0) | 1.6578 | 0.72929 | 0.0083674 |
| PE 42:10a | 0.65876 | −0.60218 | 0.03597 |
| PE 42:10b | 0.41166 | −1.2805 | 0.0012344 |
| PG (18:1/18:2) | 1.7919 | 0.8415 | 0.032264 |
| PG 40:8 | 0.57633 | −0.79503 | 0.034445 |
| PI 38:4b | 1.9426 | 0.95801 | 0.027369 |
| PS 40:8 | 0.65258 | −0.61578 | 0.02371 |
| TG 56:1 | 2.0739 | 1.0523 | 0.0015394 |
| TG 56:2 | 2.0039 | 1.0028 | 0.031988 |
| TG 56:3 | 1.8994 | 0.92556 | 0.047961 |
| TG 57:1b | 1.7678 | 0.82197 | 0.01885 |
| TG 57:2b | 1.8856 | 0.915 | 0.0070278 |
| TG 58:1 | 2.1567 | 1.1088 | 0.0066679 |
| TG 58:2 | 2.2871 | 1.1935 | 0.005624 |
| TG 58:3 | 1.6999 | 0.76543 | 0.030697 |
| TG 60:2 | 2.1669 | 1.1156 | 0.017937 |
| TG 62:14 | 0.5038 | −0.98909 | 0.0043848 |

Cardiac lipid species that are altered significantly (p≤0.5) in mice 3 days post-TAC compared to control mice 3 days post-sham surgery. Statistical analysis was performed by student t-test. Sham, n=5; 3 days TAC, n=5. Abbreviations: CL—Cardiolipins, DG—Diacylglycerols (Diglycerides), LPC—Lysophosphatidylcholines, LPE—Lysophosphatidylethanol amines, PC—Phosphatidylcholines, PE—Phosphatidyl ethanol amines, PG—Phosphatidylgylcerols, PI—Phosphatidylinositols, PS—Phosphatidylserines, TG—Triacylglycerols (Triglycerides).

TABLE 10
Cardiac lipidomic analysis 2 weeks post-TAC

| | TAC vs Sham (2 weeks) | | |
|---|---|---|---|
| Lipid Species | Fold-Change | log2(FC) | P Value |
| Cer (d18:1/23:0) | 1.7355 | 0.79538 | 0.013448 |
| Cer (d18:1/24:0) | 1.5636 | 0.64487 | 0.01203 |
| Cer (d18:1/25:0) | 2.2438 | 1.1659 | 0.0039265 |
| Cer (d18:2/23:0) | 1.6189 | 0.69502 | 0.019943 |
| Cer(d18:1/24:1) | 1.5417 | 0.62457 | 0.0018181 |
| CL (20:4_20:4_20:4_20:4) | 0.52914 | −0.91828 | 0.023837 |
| DG 38:6a | 0.62439 | −0.67949 | 0.03819 |
| DG 40:7b | 0.35129 | −1.5093 | 0.0028232 |
| LPC 16:0 | 1.6728 | 0.74227 | 0.0071848 |
| LPC 17:0 | 1.8169 | 0.86151 | 0.0051682 |
| LPC 18:1 | 2.1054 | 1.0741 | 0.0033685 |
| LPC 18:2 | 1.7875 | 0.83791 | 0.038048 |
| PC 31:0 | 1.6497 | 0.72221 | 0.0030485 |
| PC 33:0a | 1.9004 | 0.92629 | 0.00034345 |
| PC 33:0b | 1.5794 | 0.6594 | 0.0079828 |
| PC 33:2 | 2.478 | 1.3092 | 0.0038377 |
| PC 34:1 | 1.6333 | 0.70781 | 0.0018173 |
| PC 34:3 | 1.6351 | 0.70942 | 0.015013 |
| PC 34:3a | 1.574 | 0.65447 | 0.016478 |
| PC 34:3b | 1.8472 | 0.88533 | 0.017115 |
| PC 34:4a | 1.9 | 0.92598 | 0.02562 |
| PC 35:0 | 1.6809 | 0.74921 | 0.015886 |
| PC 35:1 | 2.2122 | 1.1455 | 0.00020256 |
| PC 35:3 | 2.5596 | 1.3559 | 0.0010006 |
| PC 36:2 | 1.6368 | 0.71085 | 0.021659 |
| PC 36:3 | 1.5852 | 0.66467 | 0.002131 |
| PC 36:5a | 2.0575 | 1.0409 | 0.021707 |
| PC 37:1 | 2.0764 | 1.0541 | 0.0053481 |
| PC 37:3 | 2.9677 | 1.5693 | 0.00043614 |
| PC 37:4 | 1.7764 | 0.82894 | 0.0015232 |
| PC 40:1 | 1.5589 | 0.64053 | 0.03104 |
| PC 40:2 | 1.5902 | 0.66919 | 0.046423 |
| PC 40:5a | 1.5519 | 0.63407 | 0.0063256 |
| PC 42:2 | 1.5896 | 0.66862 | 0.017231 |
| PC 42:3.1 | 1.9213 | 0.94211 | 0.011389 |
| PC 44:6 | 1.529 | 0.6126 | 0.034824 |
| PC O-34:1 | 1.8553 | 0.89164 | 0.0017079 |
| PC O-36:3 | 1.6821 | 0.75022 | 0.002287 |
| PC O-36:4b | 2.1698 | 1.1176 | 0.00049336 |
| PE O-34:3 | 1.5106 | 0.59511 | 0.043337 |
| PE (16:0/18:1) | 1.7229 | 0.78483 | 0.012491 |
| PE (16:1/18:1) | 1.9037 | 0.92882 | 0.016334 |
| PE (16:1/20:4) | 2.0352 | 1.0252 | 0.045769 |
| PE (17:0/20:4) | 2.268 | 1.1814 | 0.003017 |
| PE (18:0/20:3) | 1.5654 | 0.64651 | 0.035879 |
| PE (18:0/22:4) | 1.6638 | 0.73448 | 0.010714 |
| PE (18:1/18:1) | 1.9618 | 0.97218 | 0.0072298 |
| PE (18:1/18:2) | 1.8345 | 0.87542 | 0.016717 |
| PE (18:1/18:3) | 1.5303 | 0.61383 | 0.025447 |
| PE (18:1/20:1) | 1.651 | 0.72335 | 0.011513 |
| PE (18:1/22:0) | 1.865 | 0.89914 | 0.015727 |
| PE (18:1/22:1) | 1.8902 | 0.91855 | 0.016234 |
| PE (18:1/22:4)a | 1.6019 | 0.6798 | 0.0080857 |
| PE (18:1/24:0) | 2.6784 | 1.4214 | 0.014043 |
| PE (18:2/18:2) | 2.0115 | 1.0083 | 0.013443 |
| PE (19:1/18:0) | 1.9697 | 0.97796 | 0.0051612 |
| PE (19:1/22:6) | 1.7142 | 0.77757 | 0.0036713 |
| PE (20:0/20:4) | 1.5779 | 0.658 | 0.035559 |
| PE (22:0/20:4) | 2.1675 | 1.116 | 0.0028291 |
| PE (22:0/22:6) | 1.906 | 0.93051 | 0.020015 |
| PE (22:1/20:4) | 1.9044 | 0.92936 | 0.011444 |
| PE (P-16:0/16:0) | 1.5156 | 0.59989 | 0.013869 |
| PE 35:2 | 3.3668 | 1.7514 | 0.0015535 |
| PE 37:6b | 1.5583 | 0.63996 | 0.01644 |
| PE 39:6 | 1.534 | 0.61735 | 0.015116 |
| PE 42:2a | 2.5728 | 1.3633 | 0.0057952 |
| PE 42:2b | 2.6438 | 1.4026 | 0.0082198 |
| PG (18:1/18:2) | 1.8398 | 0.87957 | 0.011858 |
| PI 37:4 | 1.6163 | 0.69268 | 0.043344 |
| PS 40:4 | 1.8298 | 0.87167 | 0.041414 |
| SM (d18:1/15:0) | 1.5331 | 0.61646 | 0.0102 |
| SM (d18:1/23:0) | 1.5628 | 0.64415 | 0.0015624 |
| SM (d18:1/23:1) | 1.8558 | 0.89205 | 0.00065959 |
| SM (d18:1/25:1) | 1.6731 | 0.7425 | 0.0069799 |
| TG 54:8 | 0.41237 | −1.278 | 0.025095 |
| TG 56:10 | 0.44519 | −1.1675 | 0.0013206 |
| TG 56:7 | 0.63355 | −0.65847 | 0.0083299 |
| TG 57:8 | 0.62419 | −0.67994 | 0.0066512 |
| TG 57:9 | 0.58761 | −0.76708 | 0.0012086 |
| TG 58:8 | 0.66225 | −0.59456 | 0.012439 |
| TG 58:9 | 0.63031 | −0.66587 | 0.012013 |
| TG 60:11 | 0.56469 | −0.82446 | 0.025747 |
| TG 60:13 | 0.36526 | −1.453 | 0.0014332 |
| TG 60:6 | 0.66338 | −0.5921 | 0.046792 |
| TG 62:10 | 0.52748 | −0.9228 | 0.018576 |
| TG 62:12 | 0.55641 | −0.84578 | 0.047839 |
| TG 62:13 | 0.48098 | −1.0559 | 0.024872 |
| TG 62:14 | 0.44442 | −1.17 | 0.019188 |

Cardiac lipid species that are altered significantly in mice 2 weeks post-TAC compared to control mice 2 weeks post-sham surgery. Statistical analysis was performed by student t-test. Sham, n=5; 2 Weeks TAC, n=5. Abbreviations: Cer—Ceramides, CL—Cardiolipins, DG—Diacylglycerols (Diglycerides), LPC—Lysophosphatidylcholines, LPE—Phosphatidylethanolamines, PC—Phosphatidylcholines, PE—Phosphatidylethanolamines, PG—Phosphatidylgylcerols, PI—Phosphatidylinositols, PS—Phosphatidylserines, SM—Sphingomyelin, TG—Triacylglycerols (Triglycerides).

TABLE 11
Cardiac lipidomic analysis 8 weeks post-TAC

| | TAC vs Sham (8 weeks) | | |
|---|---|---|---|
| Lipid | Fold-change | log2(FC) | P value |
| Acetylcarnitine | 5.2699 | 2.3978 | 0.013385 |
| C16 acylcarnitine | 0.39722 | −1.332 | 0.015656 |
| C18 acylcarnitine | 0.37158 | −1.4283 | 0.0038484 |
| C18-hydroxy acylcarnitine | 0.43265 | −1.2087 | 0.031914 |
| Cer (d18:1/19:0) | 0.57027 | −0.81028 | 0.034479 |
| Cer (d18:1/20:0) | 0.53809 | −0.89407 | 0.0080217 |
| Cer (d18:1/22:0) | 0.40798 | −1.2934 | 0.00045156 |
| Cer (d18:1/23:0) | 0.29363 | −1.7679 | 0.00033775 |
| Cer (d18:1/24:0) | 0.31469 | −1.668 | 0.0002362 |
| CL 18:1_18:2_20:4_20:4 | 0.3023 | −1.7259 | 0.01008 |
| CL 20:4_20:4_20:4_20:4 | 0.19787 | −2.3374 | 0.0037813 |
| CL 22:6_18:2_18:2_18:2 | 0.61338 | −0.70515 | 0.027361 |
| DG 34:2 | 2.0625 | 1.0444 | 0.0025004 |
| DG 40:8 | 0.50463 | −0.98671 | 0.0052688 |
| GlcCer (d18:1/18:0) | 1.517 | 0.60123 | 0.0014594 |
| GlcCer (d18:1/24:0) | 2.082 | 1.058 | 3.96E−07 |
| LPE O-18:1 | 0.53304 | −0.90769 | 0.0019307 |
| LPE O-20:1 | 0.52729 | −0.92334 | 0.0037772 |
| PC 14:0/18:2 | 1.7186 | 0.78125 | 0.0345 |
| PC 16:0/18:3 | 1.8454 | 0.8839 | 0.0062705 |
| PC 16:1/17:1 | 2.046 | 1.0328 | 0.0028927 |
| PC 17:0/18:1 | 1.5051 | 0.58989 | 0.0025246 |
| PC 17:0/18:2 | 1.7929 | 0.8423 | 0.004207 |
| PC 17:1/18:2 | 2.5729 | 1.3634 | 0.0012561 |
| PC 18:0/18:1 | 1.6658 | 0.73619 | 0.00088519 |
| PC 18:1/19:0 | 1.6168 | 0.69316 | 0.009656 |
| PC 18:2/18:0 | 1.8007 | 0.84859 | 0.0010077 |
| PC 18:2/19:0 | 2.1481 | 1.1031 | 0.0047501 |
| PC 20:3/22:6 | 0.61028 | −0.71245 | 0.021673 |
| PC 22:6/22:5 | 0.50303 | −0.9913 | 0.014811 |
| PC 22:6/22:6 | 0.55321 | −0.8541 | 0.027903 |
| PC 34:4b | 2.5198 | 1.3333 | 0.0002232 |
| PC 36:5a | 2.6486 | 1.4052 | 0.00030862 |
| PC 42:6 | 0.64761 | −0.6268 | 0.033805 |
| PC O-16:0/18:0 | 0.4474 | −1.1604 | 0.019971 |
| PC O-16:1/18:2b | 0.44573 | −1.1658 | 0.0071225 |
| PC O-16:1/22:5 | 0.61152 | −0.70953 | 0.0035245 |
| PC O-18:0/18:2 | 0.46229 | −1.1131 | 0.036926 |
| PC O-18:0/20:4 | 0.52555 | −0.9281 | 0.028717 |
| PC O-18:0/22:5 | 0.48872 | −1.0329 | 0.0013368 |
| PC O-18:0/22:6 | 0.49768 | −1.0067 | 0.0014288 |
| PC O-18:1/22:6 | 0.46039 | −1.1191 | 0.00031615 |
| PC O-38:5b | 0.65023 | −0.62097 | 0.014018 |
| PC O-40:8a | 0.55982 | −0.83697 | 0.0054143 |
| PC O-40:8b | 0.62233 | −0.68426 | 0.0017532 |
| PC18:0/20:3b | 1.7292 | 0.79008 | 0.0050037 |
| PCe 40:6 | 0.58974 | −0.76185 | 0.015704 |
| PE 16:1/18:1 | 2.3791 | 1.2504 | 0.00044874 |
| PE 16:1/18:2 | 2.096 | 1.0677 | 0.0072473 |
| PE 18:0/18:2 | 2.3625 | 1.2403 | 3.19E−05 |
| PE 18:0/20:3 | 1.8953 | 0.92244 | 0.00036495 |
| PE 18:1/18:2 | 1.8736 | 0.90583 | 0.0020113 |
| PE 18:1/20:1 | 1.5427 | 0.62542 | 0.0050725 |
| PE 18:1/24:0 | 1.5975 | 0.67583 | 0.00029313 |
| PE 18:1/24:1 | 2.174 | 1.1203 | 0.00027743 |
| PE 19:0/22:6b | 1.5742 | 0.65461 | 0.0012101 |
| PE 22:0/20:4 | 1.7443 | 0.80262 | 0.0011894 |
| PE 22:0/22:6 | 2.221 | 1.1512 | 0.00012182 |
| PE 36:4b | 2.756 | 1.4626 | 0.0027183 |
| PE O-18:0/18:2 | 0.5367 | −0.89782 | 0.0064493 |
| PE O-18:0/22:6a | 0.64197 | −0.63942 | 0.0021005 |
| PG 16:1/20:4 | 1.9096 | 0.93323 | 0.015966 |
| PI 16:0/18:2 | 1.6281 | 0.70317 | 0.0016408 |
| PI 18:0/18:2 | 1.8879 | 0.91675 | 0.00019746 |
| PI 18:1/22:6 | 0.64415 | −0.63454 | 0.015765 |
| PS 18:0/18:2 | 1.9034 | 0.92861 | 0.00018406 |
| PS 18:4/22:4 | 1.501 | 0.58593 | 0.029003 |
| SM 17:1/24:1 | 1.6179 | 0.69412 | 0.00010267 |
| SM d18:1/25:1 | 1.5963 | 0.67477 | 2.43E−05 |
| SM d18:2/24:1 | 1.6275 | 0.70263 | 0.00022737 |
| TG 60:9 | 0.61267 | −0.70681 | 0.040345 |
| TG 62:12 | 0.39587 | −1.3369 | 0.002466 |
| TG 62:14 | 0.39693 | −1.333 | 0.0021206 |

Cardiac lipid species that are altered significantly in mice 8 weeks post-TAC compared to control mice 8 weeks post-sham surgery. Statistical analysis was performed by student t-test. Sham, n=5; 8 Weeks TAC, n=5. Abbreviations: Cer—Ceramides, CL—Cardiolipins, DG—Diacylglycerols (Diglycerides), GlcCer—Glucosylceramides, LPC—Lysophosphatidylcholines, LPE—Phosphatidylethanolamines, PC—Phosphatidylcholines, PE—Phosphatidylethanolamines, PG—Phosphatidylgylcerols, PI—Phosphatidylinositols, PS—Phosphatidylserines, SM—Sphingomyelin, TG—Triacylglycerols(Triglycerides).

TABLE 12
2D echocardiography 8 weeks post-TAC

| | CTRL-Sham | | CTRL-TAC | | HEP-FGF21^{-/-} TAC | |
|---|---|---|---|---|---|---|
| M-Mode | Mean | SEM | Mean | SEM | Mean | SEM |
| LVD(s) | 2.029 | 0.077 | 3.308**** | 0.197 | 2.271^{$$$} | 0.114 |
| LVD(d) | 3.539 | 0.0651 | 4.191** | 0.170 | 3.599^{$$} | 0.106 |
| LV Vol(s) | 13.500 | 1.283 | 45.738**** | 6.167 | 18.025^{$$$} | 2.441 |
| LV Vol(d) | 52.453 | 2.345 | 79.193** | 7.498 | 54.854^{$} | 3.865 |
| Stroke | 38.952 | 1.977 | 33.454 | 2.307 | 36.828 | 1.965 |
| Volume | | | | | | |
| EF (%) | 74.321 | 2.034 | 43.402**** | 3.396 | 67.654^{$$$$} | 2.202 |
| FS (%) | 42.666 | 1.799 | 21.353**** | 1.947 | 37.029^{$$$$} | 1.596 |
| Cardiac | 20.566 | 1.067 | 16.739 | 1.278 | 18.457 | 1.156 |
| Output | | | | | | |
| LV Mass | 97.415 | 9.695 | 157.773** | 12.429 | 114.529^{$} | 11.362 |
| LVAW(s) | 1.639 | 0.081 | 1.586 | 0.081 | 1.631 | 0.107 |
| LVAW(d) | 1.023 | 0.068 | 1.165 | 0.078 | 1.124 | 0.083 |
| LVPW(s) | 1.333 | 0.103 | 1.276 | 0.057 | 1.449 | 0.083 |
| LVPW(d) | 0.846 | 0.079 | 1.024 | 0.032 | 0.934 | 0.080 |
| Heart Rate | 527.944 | 5.496 | 499.955 | 12.917 | 500.200 | 9.771 |

Mean and SEM for echocardiography parameters measured using M-Mode short axis analysis for control (CTRL) mice subjected to sham surgery. CTRL mice subjected to TAC, and HEP-FGF21−/− mice subjected to TAC (8 weeks post TAC). n=6 Ctrl Sham, n=6 Ctrl TAC, n=6 TIEP-FGF21−/− TAC. **p<0.01 ****p<0.0001 vs CTRL-Sham; ^{$}p<0.05, ^{$$}p<0.01, ^{$$$}p<0.001, ^{$$$$}p<0.0001 vs CTRL-TAC by one-way ANOVA with Tukey Multiple comparison test.

TABLE 13
2D echocardiography 8 weeks post-TAC

| | | | | | HEP-FGF21^{-/-} | |
|---|---|---|---|---|---|---|
| | CTRL-Sham | | CTRL-TAC | | TAC | |
| B-Mode | Mean | SEM | Mean | SEM | Mean | SEM |
| EDV (μl) | 32.638 | 3.090 | 41.154 | 7.392 | 33.09 | 4.145 |
| ESV (μl) | 10.446 | 1.614 | 28.069* | 6.940 | 12.733 | 3.134 |
| SV (μl) | 22.188 | 1.947 | 13.196** | 1.817 | 20.361^{$} | 1.588 |
| FS (%) | 33.903 | 3.937 | 17.949* | 2.291 | 30.605^{$} | 3.773 |
| CO (ml/min) | 10.89 | 0.723 | 6.545** | 0.893 | 9.765^{$} | 0.842 |
| GLS | 21.855 | 3.045 | 8.730** | 2.194 | 21.325^{$} | 2.791 |
| (Peak %) | | | | | | |
| EF (%) | 68.4 | 3.385 | 34.36*** | 5.966 | 61.38^{$$} | 4.612 |
| Heart Rate | 496.666 | 20.014 | 509 | 9.774 | 479.833 | 15.330 |

Mean and SEM for echocardiography parameters measured using B-Mode long axis analysis for control (CTRL) mice subjected to sham surgery, CTRL mice subjected to TAC, and HEP-FGF21−/− mice subjected to TAC (8 weeks post TAC). n=6 Ctrl Sham, n=6 Ctrl TAC, n=6 HEP-FGF21−/− TAC. *p<0.05, **p<0.01, ****p<0.0001 vs CTRL-Sham; $p<0.05, $$p<0.01 vs CTRL-TAC by one-way ANOVA with Tukey Multiple comparison test.

TABLE 14
2D echocardiography 8 weeks post-TAC

| | CTRL-Sham | | CTRL-TAC | | CM-FGF21^{-/-} TAC | |
|---|---|---|---|---|---|---|
| M-Mode | Mean | SEM | Mean | SEM | Mean | SEM |
| LVD(s) | 1.916 | 0.080 | 2.998**** | 0.106 | 2.077^{$$$$} | 0.129 |
| LVD(d) | 3.366 | 0.083 | 3.944** | 0.097 | 3.410^{$$} | 0.116 |
| LV Vol(s) | 11.658 | 1.277 | 35.459**** | 3.045 | 14.600^{$$$$} | 2.325 |
| LV Vol(d) | 46.548 | 2.809 | 68.072** | 4.021 | 48.347^{$$} | 3.957 |
| Stroke | 34.889 | 1.619 | 32.613 | 1.685 | 33.746 | 1.770 |
| Volume | | | | | | |
| EF (%) | 75.267 | 1.300 | 48.222**** | 2.065 | 70.649^{$$$$} | 2.263 |
| FS (%) | 43.188 | 1.107 | 24.048**** | 1.231 | 39.344^{$$$$} | 1.786 |
| Cardiac | 18.447 | 0.850 | 16.590 | 0.837 | 17.378 | 0.882 |
| Output | | | | | | |
| LV Mass | 106.492 | 10.590 | 136.113 | 14.687 | 123.331 | 11.231 |
| LVAW(s) | 1.756 | 0.108 | 1.544 | 0.100 | 1.824 | 0.083 |
| LVAW(d) | 1.250 | 0.142 | 1.144 | 0.072 | 1.296 | 0.070 |
| LVPW(s) | 1.319 | 0.085 | 1.233 | 0.127 | 1.439 | 0.082 |
| LVPW(d) | 0.846 | 0.050 | 0.954 | 0.096 | 0.986 | 0.069 |
| Heart Rate | 528.890 | 3.890 | 509.356 | 10.456 | 515.531 | 6.918 |

Mean and SEM for echocardiography parameters measured using M-Mode short axis analysis control (CTRL) mice subjected to sham surgery, CTRL mice subjected to TAC, and CM-FGF21−/− mice subjected to TAC (8 weeks post TAC) (mean and SEM). n=6 for Ctrl Sham, n=6 for Ctrl TAC, n=6 for CM-FGF21−/− TAC. **p<0.01, ****p<0.0001 vs CTRL-Sham; ^{$$}p<0.01, ^{$$$$}p<0.0001 vs CTRL-TAC by one-way ANOVA with Tukey Multiple comparison test.

TABLE 15
2D echocardiography 8 weeks post-TAC

| | CTRL-Sham | | CTRL-TAC | | CM-FGF21^{-/-} TAC | |
|---|---|---|---|---|---|---|
| B-Mode | Mean | SEM | Mean | SEM | Mean | SEM |
| EDV (μl) | 29.603 | 3.295 | 43.029 | 6.827 | 29.518 | 2.990 |
| ESV (μl) | 9.095 | 1.703 | 27.978* | 6.96 | 11.088^{$} | 1.204 |
| SV (μl) | 20.505 | 1.810 | 14.881 | 1.103 | 18.425 | 1.992 |
| FS (%) | 37.455 | 3.555 | 18.802*** | 2.296 | 29.416^{$} | 1.115 |
| CO (ml/min) | 10.063 | 0.687 | 7.593 | 0.571 | 9.374 | 1.058 |
| GLS (Peak %) | 22.498 | 2.573 | 10.592** | 1.975 | 20.268^{$$} | 0.735 |
| EF (%) | 70.1666 | 3.097 | 38.388**** | 5.330 | 62.274^{$$} | 1.774 |
| Heart Rate | 497.166 | 23.597 | 509.666 | 10.210 | 502.333 | 5.981 |

Mean and SEM for echocardiography parameters measured using B-Mode long axis analysis control (CTRL) mice subjected to sham surgery, CTRL mice subjected to TAC, and CM-FGF21−/− mice subjected to TAC (8 weeks post TAC) (mean and SEM). n=6 for Ctrl Sham, n=6 for Ctrl TAC, n=6 for CM-FGF21−/− TAC. *p<0.05, **p<0.01, ****p<0.0001 vs CTRL-Sham; ^{$}p<0.05, ^{$$}p<0.01, vs CTRL-TAC by one-way ANOVA with Tukey Multiple comparison test.

TABLE 16
Upregulated cardiac transcripts in both HEP-FGF21−/− and CM-FGF21−/− mice 8 weeks post-TAC

| | | HEP-FGF21−/− TAC | | CM-FGF21−/−TAC | |
|---|---|---|---|---|---|
| | | vs Ctrl TAC | | vs Ctrl TAC | |
| Abbreviation | NAME | Fold Change | P value | Fold Change | P value |
| 3632451O06RIK | — | 0.851247514 | 0.000416561 | 0.94517666 | 1.34E−05 |
| 4430402I18RIK | — | 0.651264175 | 0.001894268 | 0.721775328 | 0.000111151 |
| 5430403G16RIK | — | 1.0524328 | 1.21E−07 | 1.17466985 | 3.04E−10 |
| 5830417I10RIK | — | 0.664237228 | 0.000320709 | 0.618665872 | 3.25E−06 |
| 9030617O03RIK | — | 0.759772174 | 4.71E−06 | 1.020700673 | 1.60E−11 |
| A2M | Alpha-2-Macroglobulin | 3.845746975 | 0.001016248 | 3.571857453 | 0.002047827 |
| AC109138.1 | — | 0.838001192 | 2.49E−05 | 0.855990716 | 4.71E−06 |
| ACOT1 | Acyl-CoA Thioesterase 1 | 1.674064526 | 1.34E−06 | 1.774736682 | 2.49E−05 |
| ADAM1A | A Disintegrin And Metalloproteinase | 0.85919673 | 0.001926559 | 0.685654155 | 0.002816513 |
| | Domain 1 (Fertilin Alpha) | | | | |
| | Pseudogene | | | | |
| ADAMTS7 | ADAM Metallopeptidase With | 0.643323532 | 0.002403314 | 1.179629848 | 2.69E−07 |
| | Thrombospondin Type 1 Motif 7 | | | | |
| ADAMTSL3 | A Disintegrin-Like And | 0.561549067 | 0.00343358 | 0.735025141 | 1.57E−06 |
| | Metalloprotease Domain With | | | | |
| | Thrombospondin Type I Motifs-Like | | | | |
| | 3 | | | | |
| ADGRG6 | Adhesion G Protein-Coupled | 1.428968767 | 0.000126449 | 1.183082366 | 2.62E−05 |
| | Receptor G6 | | | | |
| ADRA1B | Adrenoceptor Alpha 1B | 0.887147886 | 5.94E−05 | 0.909774001 | 2.92E−05 |
| AGTPBP1 | ATP/GTP Binding Carboxypeptidase | 0.503074332 | 0.002625633 | 0.736754626 | 4.44E−15 |
| | 1 | | | | |
| ALDH1L2 | Aldehyde Dehydrogenase 1 Family | 0.883819506 | 0.000255742 | 0.539448843 | 0.003065004 |
| | Member L2 | | | | |
| ALDOB | Aldolase, Fructose-Bisphosphate B | 1.888371036 | 0.00015564 | 2.238729859 | 0.001750472 |
| ANGPT1 | Angiopoietin 1 | 2.38572167 | 1.71E−05 | 1.281877098 | 2.74E−06 |
| ANKRD63 | Ankyrin Repeat Domain-Containing | 4.101665777 | 0.000306743 | 4.7581634 | 9.48E−07 |
| | Protein 63 | | | | |
| AQP4 | Aquaporin 4 | 2.270824047 | 0.000197352 | 2.90438386 | 1.80E−09 |
| ART5 | ADP-Ribosyltransferase 5 | 0.980861293 | 4.54E−07 | 0.633941437 | 0.000338422 |
| ASB4 | Ankyrin Repeat And SOCS Box | 1.215929321 | 0.001486283 | 1.262757209 | 0.000621432 |
| | Containing 4 | | | | |
| ATCAY | ATCAY Kinesin Light Chain | 1.158924964 | 1.49E−14 | 0.833902501 | 2.07E−05 |
| | Interacting Caytaxin | | | | |
| AUTS2 | Activator Of Transcription And | 0.672183266 | 3.03E−05 | 0.526691918 | 3.76E−06 |
| | Developmental Regulator AUTS2 | | | | |
| BBS2 | Bardet-Biedl Syndrome 2 | 0.569221039 | 0.000320888 | 0.542598996 | 0.00189363 |
| BC030499 | — | 0.535244464 | 0.000844231 | 0.601741213 | 0.001342814 |
| BCL2L11 | Bcl-2-Like Protein 11 | 0.623023254 | 0.003468805 | 0.867567722 | 1.35E−05 |
| CAMK2A | Calcium/Calmodulin Dependent | 0.513138018 | 0.000297811 | 0.570351544 | 4.15E−06 |
| | Protein Kinase II Alpha | | | | |
| CAR3 | Carbonate Dehydratase III | 6.371636982 | 3.45E−08 | 2.662998926 | 9.19E−10 |
| CARNS1 | Carnosine Synthase 1 | 0.838001192 | 2.49E−05 | 0.855990716 | 4.71E−06 |
| CCDC171 | Coiled-Coil Domain Containing 171 | 0.885130485 | 0.000176962 | 0.817890778 | 3.26E−05 |
| CELSR2 | Cadherin EGF LAG Seven-Pass G- | 0.955904836 | 0.000248366 | 1.055385252 | 1.61E−07 |
| | Type Receptor 2 | | | | |
| CELSR3 | Cadherin EGF LAG Seven-Pass G- | 1.385793799 | 8.08E−06 | 1.098015553 | 0.005113801 |
| | Type Receptor 3 | | | | |
| CES1D | Carboxylesterase 1D | 1.283825365 | 5.27E−05 | 1.711111415 | 3.84E−11 |
| CFD | Complement Factor D | 7.658685772 | 5.60E−10 | 2.661434913 | 0.001037677 |
| CLCN1 | Chloride Voltage-Gated Channel 1 | 1.779250859 | 4.16E−05 | 1.804457722 | 0.003286602 |
| CLEC3B | C-Type Lectin Domain Family 3 | 0.899024218 | 0.000992677 | 1.055127578 | 0.000147041 |
| | Member B | | | | |
| CMSS1 | Cms1 Ribosomal Small Subunit | 0.67407996 | 0.000254455 | 0.882963062 | 1.49E−06 |
| | Homolog | | | | |
| COL6A6 | Collagen Type VI Alpha 6 Chain | 1.275043477 | 5.89E−06 | 0.988030407 | 3.79E−05 |
| CRHR2 | Corticotropin Releasing Hormone | 0.842266785 | 4.90E−07 | 0.856425702 | 1.16E−06 |
| | Receptor 2 | | | | |
| CTH | Cystathionase (Cystathionine | 0.872374695 | 0.002710953 | 0.963908568 | 0.001219459 |
| | Gamma-Lyase) | | | | |
| CTLA4 | Cytotoxic T-Lymphocyte Associated | 1.569873993 | 8.10E−05 | 1.859478734 | 2.10E−05 |
| | Protein 4 | | | | |
| CYB5R2 | Cytochrome B5 Reductase 2 | 1.022995306 | 2.89E−06 | 0.752730555 | 0.000209435 |
| DHRS7C | Dehydrogenase/Reductase 7C | 0.758371068 | 1.89E−06 | 0.752343965 | 7.34E−07 |
| DIXDC1 | DIX Domain Containing 1 | 0.507854879 | 2.82E−05 | 0.554866647 | 0.000199762 |
| DPF3 | Double PHD Fingers 3 | 0.74562825 | 0.000162383 | 0.909326417 | 2.14E−05 |
| DPP4 | Dipeptidyl Peptidase 4 | 1.572952891 | 9.61E−06 | 0.819736289 | 0.002376097 |
| EFNB3 | Ephrin B3 | 0.688248643 | 0.001320934 | 0.79417096 | 7.97E−05 |
| EGFLAM | EGF Like, Fibronectin Type III And | 0.627103389 | 0.001383647 | 0.799505027 | 0.002356523 |
| | Laminin G Domains | | | | |
| EMILIN2 | Elastin Microfibril Interfacer 2 | 0.989234366 | 1.01E−06 | 1.120827324 | 2.31E−10 |
| ENPP2 | Ectonucleotide | 1.685526752 | 9.03E−07 | 1.444196418 | 6.12E−07 |
| | Pyrophosphatase/Phosphodiesterase | | | | |
| | 2 | | | | |
| EPB41L4B | Erythrocyte Membrane Protein Band | 1.784429725 | 0.000771058 | 2.168927712 | 8.05E−08 |
| | 4.1 Like 4B | | | | |
| EPHA7 | Ephrin Type-A Receptor 7 | 1.169781396 | 3.51E−07 | 1.01579297 | 1.00E−07 |
| ERBB4 | Erb-B2 Receptor Tyrosine Kinase 4 | 1.329718021 | 0.000896929 | 0.592872776 | 0.003484907 |
| ERICH3 | Glutamate Rich 3 | 1.022501205 | 0.002386898 | 1.263092698 | 0.000801258 |
| FIGN | Fidgetin, Microtubule Severing | 1.234190643 | 5.44E−11 | 1.163447314 | 6.83E−08 |
| | Factor | | | | |
| FKBP2 | FKBP Prolyl Isomerase 2 | 0.518158587 | 0.00211198 | 0.589199361 | 0.001739177 |
| GABRG3 | Gamma-Aminobutyric Acid Type A | 1.671866063 | 0.000303113 | 1.094553273 | 0.004081104 |
| | Receptor Subunit Gamma3 | | | | |
| GABRR2 | Gamma-Aminobutyric Acid Type A | 1.502166072 | 0.00195487 | 2.155939059 | 1.41E−06 |
| | Receptor Subunit Rho2 | | | | |
| GFOD2 | Gfo/Idh/MocA-Like Oxidoreductase | 0.588099907 | 9.64E−05 | 0.662733758 | 2.78E−05 |
| | Domain Containing 2 | | | | |
| GFRA4 | GDNF Family Receptor Alpha 4 | 1.15168509 | 0.000753099 | 1.686090639 | 3.20E−08 |
| GGCX | Gamma-Glutamyl Carboxylase | 0.535898506 | 7.92E−05 | 0.638374483 | 1.94E−06 |
| GM10118 | — | 0.794044532 | 0.00086615 | 1.24492891 | 1.68E−14 |
| GM12689 | — | 2.125665603 | 0.002128447 | 2.523222268 | 5.93E−05 |
| GM13622 | — | 1.662329141 | 0.003132243 | 1.59316536 | 0.001797453 |
| GM15712 | — | 1.113153899 | 5.72E−05 | 1.15150876 | 0.000487028 |
| GM18448 | — | 1.137324835 | 0.000205644 | 1.09801021 | 6.73E−05 |
| GM18706 | — | 2.21516398 | 0.000400834 | 2.458676375 | 7.27E−05 |
| GM2004 | — | 0.981607587 | 0.003172021 | 1.052082856 | 0.001650644 |
| GM20696 | — | 0.89098386 | 4.85E−05 | 0.720326445 | 0.001439367 |
| GM2223 | — | 0.63682177 | 0.002897822 | 0.722447268 | 0.000434699 |
| GM26992 | — | 5.346649721 | 0.002972298 | 6.238509706 | 2.03E−17 |
| GM42584 | — | 2.146440272 | 2.29E−06 | 1.785144384 | 0.000226546 |
| GM42647 | — | 4.998076606 | 3.86E−05 | 5.032515241 | 0.000528382 |
| GM43841 | — | 1.132548037 | 0.001411043 | 1.849097969 | 0.004085473 |
| GM44048 | — | 3.595270035 | 3.10E−05 | 4.165980995 | 2.20E−06 |
| GM44578 | — | 0.567460906 | 0.001409012 | 0.620668192 | 0.000527317 |
| GM4461 | — | 0.583745138 | 0.002664868 | 0.688802018 | 0.002846816 |
| GM45531 | — | 1.429296398 | 0.000163004 | 1.015529804 | 7.39E−05 |
| GM6644 | — | 2.79237322 | 0.000502843 | 3.183431196 | 0.002109964 |
| GPD2 | Glycerol-3-Phosphate | 0.705563435 | 4.20E−05 | 0.505621151 | 0.000122163 |
| | Dehydrogenase 2 | | | | |
| GRAMD4 | GRAM Domain Containing 4 | 0.743813525 | 2.84E−05 | 0.524076069 | 0.001328299 |
| GSTP2 | Eukaryotic Peptide Chain Release | 1.042777211 | 0.000163299 | 1.234066432 | 1.04E−05 |
| | Factor GTP-Binding Subunit ERF3B | | | | |
| GYPC | Glycophorin C | 0.558232322 | 6.61E−08 | 0.636893163 | 0.000142242 |
| HILS1 | H1.9 Linker Histone, Pseudogene | 1.152556408 | 0.001832128 | 1.482891235 | 1.93E−05 |
| HIST2H2BE | H2B Clustered Histone 21 | 0.628826185 | 0.000977182 | 0.514757674 | 0.000169447 |
| HMGN5 | High Mobility Group Nucleosome | 0.749978003 | 2.12E−08 | 0.54463725 | 0.00010257 |
| | Binding Domain 5 | | | | |
| HNMT | Histamine N-Methyltransferase | 0.858515674 | 4.47E−07 | 1.150627055 | 3.68E−08 |
| HPF1 | Histone PARylation Factor 1 | 0.584640939 | 8.97E−06 | 0.551875914 | 0.000773561 |
| HSD17B14 | Hydroxysteroid 17-Beta | 1.074991308 | 0.000133261 | 1.180463247 | 0.000111265 |
| | Dehydrogenase 14 | | | | |
| HTRA1 | High-Temperature Requirement A | 0.618671245 | 6.27E−08 | 0.621803805 | 3.73E−06 |
| | Serine Peptidase 1 | | | | |
| IL15 | Interleukin 15 | 0.560573661 | 7.50E−05 | 0.803109545 | 0.000227595 |
| IRF2BP2 | Interferon Regulatory Factor 2 | 0.514799846 | 1.66E−06 | 0.530780266 | 1.75E−06 |
| | Binding Protein 2 | | | | |
| ITGB6 | Integrin Subunit Beta 6 | 0.784847697 | 1.27E−05 | 0.769471643 | 1.36E−05 |
| KCNA1 | Potassium Voltage-Gated Channel | 1.451141132 | 0.001916966 | 1.553157438 | 0.000341518 |
| | Subfamily A Member 1 | | | | |
| KCND2 | Potassium Voltage-Gated Channel | 1.055745852 | 4.87E−07 | 1.025358228 | 4.21E−06 |
| | Subfamily D Member 2 | | | | |
| KCNH2 | Potassium Voltage-Gated Channel | 0.807681555 | 3.07E−06 | 0.854028614 | 2.11E−07 |
| | Subfamily H Member 2 | | | | |
| KCNJ2 | Potassium Inwardly Rectifying | 0.684487955 | 5.39E−07 | 0.79182193 | 1.00E−06 |
| | Channel Subfamily J Member 2 | | | | |
| KCNJ5 | Potassium Inwardly Rectifying | 1.17760339 | 0.000692382 | 1.124261547 | 6.95E−19 |
| | Channel Subfamily J Member 5 | | | | |
| KCNQ1 | Potassium Voltage-Gated Channel | 0.501623708 | 0.000404408 | 0.546589679 | 3.20E−05 |
| | Subfamily Q Member 1 | | | | |
| KCNV2 | Potassium Voltage-Gated Channel | 1.157836406 | 8.59E−05 | 2.094071001 | 7.43E−13 |
| | Modifier Subfamily V Member 2 | | | | |
| KDM6A | Lysine Demethylase 6A | 0.720986544 | 0.0007635 | 1.021688183 | 1.15E−25 |
| KIF6 | Kinesin Family Member 6 | 2.022502321 | 1.30E−05 | 1.70626405 | 0.000532823 |
| KLHL13 | Kelch Like Family Member 13 | 1.003733068 | 3.69E−05 | 0.605777117 | 6.38E−05 |
| KLHL38 | Kelch Like Family Member 38 | 0.716295198 | 5.25E−07 | 0.611241965 | 5.67E−05 |
| LAMB3 | Laminin Subunit Beta 3 | 0.708676224 | 1.68E−05 | 1.014059717 | 1.13E−08 |
| LAT2 | Linker For Activation Of T Cells | 0.678816137 | 0.002062504 | 0.69278871 | 0.000242047 |
| | Family Member 2 | | | | |
| LGALS4 | Galectin 4 | 0.924986876 | 2.87E−05 | 1.29439062 | 1.29E−07 |
| LRP4 | LDL Receptor Related Protein 4 | 0.547266481 | 0.000880124 | 0.612470671 | 4.14E−07 |
| LRRC15 | Leucine Rich Repeat Containing 15 | 0.838442728 | 0.000362774 | 1.209152436 | 2.31E−14 |
| LRRTM3 | Leucine Rich Repeat | 0.628535563 | 0.003081245 | 0.878177031 | 3.07E−05 |
| | Transmembrane Neuronal 3 | | | | |
| LSAMP | Limbic System Associated | 0.699852803 | 0.000191932 | 0.618638973 | 6.56E−05 |
| | Membrane Protein | | | | |
| MAOB | Monoamine Oxidase B | 0.821940278 | 1.71E−07 | 0.665597502 | 0.000245859 |
| MED12L | Mediator Complex Subunit 12L | 1.077742617 | 7.21E−07 | 1.176169826 | 3.63E−07 |
| MEETAP1D | Methionyl Aminopeptidase Type 1D, | 1.05703847 | 2.37E−23 | 0.839765567 | 2.41E−06 |
| | Mitochondrial | | | | |
| MGL2 | Macrophage Galactose N-acetyl- | 0.978281282 | 4.83E−05 | 0.953157746 | 0.000550247 |
| | galactosamine Specific Lectin 2 | | | | |
| MME | Membrane Metalloendopeptidase | 0.624635536 | 4.09E−06 | 0.688573446 | 0.001658869 |
| MMP15 | Matrix Metallopeptidase 15 | 0.668011977 | 2.40E−05 | 0.602017865 | 1.08E−05 |
| MRGPRH | MAS-related GPR, member H | 1.170115049 | 0.000654489 | 1.559524348 | 2.22E−07 |
| MTERF4 | Mitochondrial Transcription | 0.686128629 | 3.27E−06 | 0.749937043 | 5.32E−08 |
| | Termination Factor 4 | | | | |
| MYRF | Myelin Regulatory Factor | 0.825870013 | 4.32E−05 | 0.619151325 | 0.003903726 |
| NCKAP5 | NCK Associated Protein 5 | 0.697649689 | 4.86E−09 | 0.68553499 | 1.46E−06 |
| NEGR1 | Neuronal Growth Regulator 1 | 0.806708016 | 0.000187024 | 0.809632213 | 7.32E−05 |
| NHSL1 | NHS-Like Protein 1 | 0.713832007 | 4.54E−11 | 0.867696237 | 8.07E−08 |
| NOS1AP | Nitric Oxide Synthase 1 Adaptor | 0.879005733 | 0.000275404 | 0.793576226 | 0.00307176 |
| | Protein | | | | |
| P2RY14 | Purinergic Receptor P2Y14 | 0.723129955 | 0.000730286 | 0.882525405 | 0.00062582 |
| P3H4 | Prolyl 3-Hydroxylase Family | 0.664338353 | 0.001243164 | 0.599506489 | 0.00389029 |
| | Member 4 (Inactive) | | | | |
| PCDHGA12 | Protocadherin Gamma Subfamily A, | 0.61272006 | 3.50E−06 | 0.689824866 | 7.96E−07 |
| | 12 | | | | |
| PDP2 | Pyruvate Dehydrogenase | 0.592289464 | 0.00024693 | 1.109545979 | 4.59E−09 |
| | Phosphatase Catalytic Subunit 2 | | | | |
| PDZRN4 | PDZ Domain Containing Ring Finger | 2.636657289 | 2.67E−06 | 1.963396214 | 0.000673448 |
| | 4 | | | | |
| PEX6 | Peroxisomal Biogenesis Factor 6 | 0.629338288 | 0.000458214 | 0.761206859 | 2.22E−05 |
| PFKFB1 | 6-Phosphofructo-2-Kinase/Fructose- | 2.028578663 | 2.69E−14 | 2.027231897 | 6.24E−13 |
| | 2,6-Biphosphatase 1 | | | | |
| PHKG1 | Phosphorylase Kinase Catalytic | 1.108981298 | 1.89E−06 | 0.916931553 | 1.65E−11 |
| | Subunit Gamma 1 | | | | |
| PITPNM3 | Phosphatidylinositol Transfer | 1.014248713 | 0.001659779 | 1.038732615 | 0.000676799 |
| | Protein, Membrane-Associated 3 | | | | |
| PLA2G4E | Cytosolic Phospholipase A2 Epsilon | 0.656112012 | 0.000207827 | 0.83181065 | 0.000137319 |
| PLAG1 | Pleiomorphic Adenoma Gene 1 | 0.686455519 | 3.02E−05 | 0.823337875 | 5.12E−08 |
| PLAGL1 | PLAG1 Like Zinc Finger 1 | 2.003688164 | 1.52E−06 | 1.130025968 | 0.000556718 |
| PLEKHA5 | Pleckstrin Homology Domain | 0.66066903 | 6.07E−07 | 0.783965449 | 7.41E−07 |
| | Containing A5 | | | | |
| PLXNB1 | Plexin B1 | 0.748024078 | 1.17E−05 | 1.176953847 | 3.33E−15 |
| PPIP5K2 | Diphosphoinositol Pentakisphosphate | 0.655786463 | 1.12E−06 | 1.054969411 | 1.99E−15 |
| | Kinase 2 | | | | |
| PPM1K | Protein Phosphatase, Mg2+/Mn2+ | 0.549972625 | 8.74E−07 | 0.977857907 | 2.30E−28 |
| | Dependent 1K | | | | |
| PPP1R10 | Protein Phosphatase 1 Regulatory | 0.995868636 | 8.84E−12 | 0.655929932 | 7.91E−06 |
| | Subunit 10 | | | | |
| PRRG1 | Proline Rich And Gla Domain 1 | 0.655804907 | 0.000149333 | 0.514010358 | 0.001347483 |
| PTTG1 | PTTG1 Regulator Of Sister | 0.557525609 | 0.002468616 | 0.774172919 | 0.001581885 |
| | Chromatid Separation, Securin | | | | |
| QSOX2 | Quiescin Sulfhydryl Oxidase 2 | 0.52137215 | 0.000111706 | 0.837498259 | 3.26E−12 |
| RAB11FIP1 | RAB11 Family Interacting Protein 1 | 0.775568568 | 0.002529218 | 0.617078782 | 0.006012682 |
| RARB | Retinoic Acid Receptor Beta | 0.569580345 | 0.000290515 | 0.787362431 | 1.34E−08 |
| RBFOX1 | RNA Binding Fox-1 Homolog 1 | 0.888127853 | 3.90E−11 | 0.916426786 | 8.84E−08 |
| RETNLA | Resistin Like Alpha | 2.688490446 | 0.000105433 | 2.034916031 | 0.000333019 |
| RGS17 | Regulator Of G Protein Signaling 17 | 1.076791156 | 0.000587243 | 1.030713998 | 0.000132858 |
| RGS2 | Regulator Of G Protein Signaling 2 | 1.529432096 | 1.67E−15 | 1.59370535 | 2.93E−20 |
| RGS6 | Regulator Of G Protein Signaling 6 | 1.240058068 | 0.000140699 | 0.702843338 | 0.003807361 |
| RGS7 | Regulator Of G Protein Signaling 7 | 1.596360323 | 4.37E−05 | 0.799582086 | 0.00034805 |
| RNF207 | Ring Finger Protein 207 | 0.751518105 | 3.31E−09 | 0.820116311 | 7.63E−11 |
| RP1 | Oxygen-Regulated Protein 1 | 3.980687441 | 2.43E−05 | 2.76331047 | 7.51E−06 |
| RP23-203D1.4 | — | 1.614864033 | 0.002206741 | 1.820101442 | 0.001627466 |
| RP23-269K2.2 | — | 1.603977808 | 0.000555616 | 1.88424287 | 1.43E−06 |
| RP24-559D16.4 | — | 0.910024607 | 2.94E−05 | 1.017476679 | 5.01E−05 |
| RTN2 | Reticulon 2 | 0.513078223 | 7.14E−05 | 0.536607117 | 0.000152159 |
| SBK2 | SH3 Domain Binding Kinase Family | 4.713444587 | 0.000152275 | 2.444148865 | 0.000982444 |
| | Member 2 | | | | |
| SBK3 | SH3 Domain Binding Kinase Family | 6.527648564 | 5.27E−07 | 1.754917813 | 7.94E−06 |
| | Member 3 | | | | |
| SCARA5 | Scavenger Receptor Class A Member | 0.592933628 | 0.000704004 | 0.539453589 | 0.003542784 |
| | 5 | | | | |
| SELENBP1 | Selenium Binding Protein 1 | 0.664753871 | 2.56E−07 | 0.772248909 | 7.61E−11 |
| SETBP1 | SET Binding Protein 1 | 0.664278922 | 4.46E−09 | 0.839150841 | 7.31E−07 |
| SHISA6 | Shisa Family Member 6 | 1.827517224 | 6.51E−08 | 1.476331923 | 9.56E−05 |
| SLC16A7 | Solute Carrier Family 16 Member 7 | 0.890209312 | 0.000587829 | 0.890907549 | 5.43E−07 |
| SLC22A3 | Solute Carrier Family 22 Member 3 | 1.346467037 | 1.18E−08 | 0.779807022 | 0.00233208 |
| SLC26A3 | Solute Carrier Family 26 Member 3 | 0.670095906 | 1.53E−05 | 0.636723164 | 9.52E−05 |
| SLC26A6 | Solute Carrier Family 26 Member 6 | 0.845696536 | 2.46E−06 | 0.885405364 | 9.36E−06 |
| SLC9A2 | Solute Carrier Family 9 Member A2 | 1.518243513 | 0.000545473 | 1.619245171 | 9.92E−05 |
| SLIT2 | Slit Guidance Ligand 2 | 0.756814971 | 0.000190956 | 0.783260615 | 1.96E−05 |
| SMIM5 | Small Integral Membrane Protein 5 | 0.699149734 | 0.002018382 | 0.89561129 | 0.000587492 |
| SNED1 | Sushi, Nidogen And EGF Like | 1.133374837 | 3.45E−08 | 1.239559082 | 3.46E−27 |
| | Domains 1 | | | | |
| SORT1 | Sortilin 1 | 1.676791438 | 0.000704796 | 0.720612349 | 1.59E−06 |
| SOX6 | SRY-Box Transcription Factor 6 | 0.541197531 | 0.000184402 | 0.672805975 | 1.00E−05 |
| SPSB1 | SplA/Ryanodine Receptor Domain | 0.813232338 | 3.42E−10 | 0.616628876 | 1.73E−06 |
| | And SOCS Box Containing 1 | | | | |
| SPTA1 | Spectrin Alpha, Erythrocytic 1 | 0.948068134 | 1.30E−06 | 1.019711265 | 7.05E−13 |
| ST6GALNAC3 | ST6 N-Acetylgalactosaminide | 1.090800008 | 1.44E−07 | 0.805694304 | 0.000186234 |
| | Alpha-2,6-Sialyltransferase 3 | | | | |
| STARD10 | StAR Related Lipid Transfer Domain | 3.29102496 | 0.000136252 | 0.799533738 | 0.000224632 |
| | Containing 10 | | | | |
| STAT5A | Signal Transducer And Activator Of | 0.624772318 | 3.38E−05 | 0.712867692 | 6.38E−06 |
| | Transcription 5A | | | | |
| SYDE2 | Synapse Defective Rho GTPase | 1.031486526 | 7.19E−11 | 1.010978593 | 2.87E−08 |
| | Homolog 2 | | | | |
| SYN3 | Synapsin III | 1.148306001 | 1.86E−05 | 0.873931207 | 0.000540733 |
| SYT3 | Synaptotagmin 3 | 1.970108048 | 4.43E−05 | 1.626297241 | 0.003441995 |
| SYT7 | Synaptotagmin 7 | 0.639258882 | 7.85E−06 | 0.747790208 | 2.09E−07 |
| TBC1D10C | TBC1 Domain Family Member 10C | 1.258235809 | 9.95E−05 | 1.468473945 | 2.31E−07 |
| TBC1D4 | TBC1 Domain Family Member 4 | 0.624818235 | 1.74E−05 | 0.573768466 | 7.33E−07 |
| TBX5 | T-Box Transcription Factor 5 | 1.675489948 | 0.001259701 | 0.882978179 | 4.34E−09 |
| TET1 | Tet Methylcytosine Dioxygenase 1 | 0.886098154 | 1.42E−06 | 0.723213774 | 5.55E−05 |
| TGFBR3L | Transforming Growth Factor Beta | 1.159214665 | 0.001598027 | 1.090162917 | 0.001898716 |
| | Receptor 3 Like | | | | |
| TMEM29 | Transmembrane protein 29 | 0.599177388 | 0.001293231 | 0.582862318 | 0.002129747 |
| TMEM35A | Transmembrane Protein 35A | 1.730315281 | 2.05E−07 | 1.86594929 | 8.82E−12 |
| TMEM56 | Transmembrane Protein 56 | 1.638489475 | 6.24E−06 | 1.410053232 | 8.28E−05 |
| | (TMEM56) Pseudogene | | | | |
| TMEM82 | Transmembrane Protein 82 | 0.736828763 | 0.00266789 | 1.016392966 | 0.001144403 |
| TPPP | Tubulin Polymerization Promoting | 0.835332715 | 8.40E−07 | 1.080526821 | 6.65E−11 |
| | Protein | | | | |
| TRIM7 | Tripartite Motif Containing 7 | 0.725587916 | 2.53E−05 | 0.718736806 | 1.01E−09 |
| UCP3 | Uncoupling Protein 3 | 1.282371881 | 1.17E−10 | 1.423802127 | 1.43E−10 |
| UPK3B | Uroplakin 3B | 5.758665291 | 1.20E−09 | 2.691237532 | 0.001300094 |
| WHRN | Whirlin | 0.984331928 | 0.002855627 | 1.2162343 | 4.36E−09 |
| WNK2 | WNK Lysine Deficient Protein | 0.647467599 | 2.23E−05 | 0.792807222 | 2.89E−06 |
| | Kinase 2 | | | | |
| WNT5A | Wnt Family Member 5A | 0.76747999 | 6.31E−05 | 0.557618417 | 0.000747682 |
| ZFP395 | Zinc Finger Protein 395 | 0.754116996 | 2.98E−08 | 0.627947549 | 0.00017988 |
| ZFP612 | Zinc Finger Protein 612 | 0.921991788 | 8.68E−11 | 1.121620662 | 2.33E−13 |
| ZFP949 | Zinc Finger Protein 949 | 0.807406488 | 0.000795743 | 0.72370849 | 0.000451072 |

Statistically significant upregulated transcripts that are common in TIEP-FGF21^{-/-} and CM-FGF21^{-/-} mice with TAC for 8 weeks compared to respective controls that were subjected to TAC.

TABLE 17
Downregulated cardiac transcripts in both HEP-FGF21−/− and CM-FGF21−/− mice 8 weeks post-TAC

| | | HEP-FGF21^{-/-} TAC vs Ctrl TAC | | CM-FGF21^{-/-} TAC vs Ctrl TAC | |
|---|---|---|---|---|---|
| Abbreviation | Name | Log2 Fold Change | p-value | Log2 Fold Change | p-value |
| 1190002N15RIK | — | −0.622626175 | 1.61E−09 | −0.790487842 | 1.79E−07 |
| 1500009L16RIK | — | −1.062577316 | 3.95E−05 | −0.998078023 | 5.32E−07 |
| 2010111I01RIK | — | −0.869933459 | 2.39E−18 | −0.723801946 | 5.24E−09 |
| A4GALT | Alpha 1,4- | −1.016768139 | 7.40E−05 | −1.109739202 | 0.000370375 |
| | Galactosyltransferase | | | | |
| ABCB4 | ATP Binding Cassette | −0.630497142 | 5.74E−05 | −0.789756611 | 2.53E−07 |
| | Subfamily B Member 4 | | | | |
| ABRA | Actin Binding Rho | −2.250096324 | 1.64E−44 | −2.082349003 | 2.95E−08 |
| | Activating Protein | | | | |
| ACTA1 | Actin Alpha 1 | −2.09415707 | 2.85E−06 | −3.099526395 | 0.000117848 |
| ADAMTS20 | A Disintegrin And | −1.831560901 | 1.88E−05 | −2.186486182 | 5.60E−07 |
| | Metalloproteinase With | | | | |
| | Thrombospondin Motifs 20 | | | | |
| ADCY7 | Adenylate Cyclase 7 | −0.511190011 | 0.000138935 | −0.92315235 | 1.16E−11 |
| AKAP2 | A-Kinase Anchoring Protein | −0.745436195 | 3.62E−12 | −0.998412247 | 2.83E−13 |
| | 2 | | | | |
| ANGPTL7 | Angiopoietin Like 7 | −0.880232491 | 0.002796324 | −0.978816124 | 0.000107154 |
| ANKRD1 | Ankyrin Repeat Domain 1 | −0.954997157 | 0.001443489 | −1.612649652 | 0.006368672 |
| ANKRD23 | Ankyrin Repeat Domain 23 | −0.684549941 | 3.51E−05 | −1.309004558 | 0.000576987 |
| AQP8 | Aquaporin 8 | −1.678064796 | 0.000777205 | −2.689240792 | 0.000325003 |
| ARHGAP11A | Rho GTPase Activating | −0.902011985 | 4.35E−05 | −0.842296937 | 0.001402771 |
| | Protein 11A | | | | |
| ARHGAP9 | Rho GTPase Activating | −0.692575892 | 0.000547434 | −0.889039707 | 3.67E−06 |
| | Protein 9 | | | | |
| ASPN | Asporin | −0.80412636 | 2.67E−07 | −1.270680457 | 8.59E−22 |
| ASS1 | Argininosuccinate Synthase | −1.024946087 | 5.09E−05 | −0.887929799 | 0.002351452 |
| | 1 | | | | |
| ATF3 | Cyclic AMP-Dependent | −2.369706101 | 8.12E−31 | −2.086339245 | 2.65E−06 |
| | Transcription Factor ATF-3 | | | | |
| BAG2 | BCL2 Associated | −0.638162789 | 4.19E−10 | −0.700440941 | 4.12E−11 |
| | Athanogene 2 | | | | |
| BCL2 | BCL2 Apoptosis Regulator | −0.799456343 | 1.09E−05 | −0.765599927 | 2.84E−05 |
| BDH1 | 3-Hydroxybutyrate | −0.786378808 | 1.83E−09 | −0.968615916 | 7.56E−05 |
| | Dehydrogenase 1 | | | | |
| BEST3 | Bestrophin 3 | −0.690044255 | 6.29E−06 | −0.891901441 | 2.70E−05 |
| BEX1 | Brain Expressed X-Linked 1 | −1.2482372 | 0.000261923 | −1.229451451 | 0.00117304 |
| BGN | Biglycan | −0.516930063 | 0.001455164 | −1.158170209 | 2.94E−07 |
| CAPG | Capping Actin Protein, | −0.563244319 | 0.002620844 | −0.794343001 | 9.70E−05 |
| | Gelsolin Like | | | | |
| CARHSP1 | Calcium Regulated Heat | −0.965001014 | 1.54E−10 | −1.120635508 | 9.32E−14 |
| | Stable Protein 1 | | | | |
| CCND1 | Cyclin D1 | −0.720286554 | 6.19E−08 | −0.729802773 | 1.00E−18 |
| CCND2 | Cyclin D2 | −0.53538094 | 0.000127842 | −0.728605697 | 3.25E−08 |
| CD44 | CD44 Molecule | −0.648958068 | 0.002037469 | −0.884389959 | 5.80E−09 |
| CDKL2 | Cyclin Dependent Kinase | −0.860612944 | 5.24E−05 | −0.985152699 | 6.82E−09 |
| | Like 2 | | | | |
| CDV3 | Carnitine Deficiency- | −0.572836756 | 3.69E−05 | −0.785542611 | 0.002326387 |
| | Associated Gene Expressed | | | | |
| | In Ventricle 3 | | | | |
| CHODL | Chondrolectin | −4.169217097 | 1.44E−07 | −5.764255081 | 4.21E−09 |
| CILP | Cartilage Intermediate Layer | −1.773885825 | 0.000195566 | −3.069962748 | 7.39E−11 |
| | Protein | | | | |
| CLIC5 | Chloride Intracellular | −0.763068583 | 2.09E−07 | −0.809002247 | 1.98E−08 |
| | Channel 5 | | | | |
| CMKLR1 | Chemerin Chemokine-Like | −0.703612753 | 8.66E−09 | −0.635955354 | 5.57E−07 |
| | Receptor 1 | | | | |
| COL11A1 | Collagen Type XI Alpha 1 | −2.579911213 | 9.42E−05 | −1.956179149 | 0.005519875 |
| | Chain | | | | |
| COL12A1 | Collagen Type XII Alpha 1 | −1.988037285 | 1.23E−05 | −3.151512396 | 8.22E−32 |
| | Chain | | | | |
| COL1A2 | Collagen Type I Alpha 2 | −0.567713465 | 0.000569414 | −1.197595254 | 1.62E−19 |
| | Chain | | | | |
| COL5A2 | Collagen Type V Alpha 2 | −0.612939677 | 0.000103486 | −1.129970234 | 5.02E−13 |
| | Chain | | | | |
| COL8A1 | Collagen Type VIII Alpha 1 | −1.351775792 | 0.00029141 | −1.788724835 | 3.46E−06 |
| | Chain | | | | |
| COMT | Catechol-O- | −1.03585162 | 0.000156471 | −0.96017393 | 0.002025969 |
| | Methyltransferase | | | | |
| CPXM2 | Carboxypeptidase X, M14 | −1.404672181 | 0.000379964 | −2.106171822 | 0.0004347 |
| | Family Member 2 | | | | |
| CRISPLD1 | Cysteine Rich Secretory | −1.064508121 | 4.70E−06 | −1.317463173 | 1.50E−07 |
| | Protein LCCL Domain | | | | |
| | Containing 1 | | | | |
| CYB561 | Cytochrome B561 | −0.603388029 | 9.82E−05 | −0.860206758 | 5.24E−06 |
| CYS1 | Cystin 1 | −1.439767139 | 0.000573813 | −0.689528752 | 0.000555794 |
| CYSTM1 | Cysteine Rich | −0.73000941 | 2.50E−08 | −0.718358824 | 4.51E−06 |
| | Transmembrane Module | | | | |
| | Containing 1 | | | | |
| D630003M21RIK | | −0.800013835 | 9.93E−05 | −0.596637064 | 0.00032062 |
| DBN1 | Drebrin 1 | −1.130861863 | 2.16E−05 | −1.565105198 | 2.94E−07 |
| DDAH1 | Dimethylarginine | −0.731528439 | 7.12E−05 | −0.957854153 | 1.45E−08 |
| | Dimethylaminohydrolase 1 | | | | |
| DNAJA4 | DnaJ Heat Shock Protein | −0.521452392 | 6.98E−10 | −0.63745448 | 4.95E−10 |
| | Family (Hsp40) Member A4 | | | | |
| DUSP27 | Serine/Threonine/Tyrosine | −0.570003771 | 4.52E−06 | −0.660842503 | 3.27E−06 |
| | Interacting Like 2 | | | | |
| | (STYXL2) | | | | |
| EDN3 | Endothelin 3 | −1.646825513 | 1.11E−06 | −2.258581626 | 0.001877611 |
| ELL2 | Elongation Factor For RNA | −0.890852201 | 8.61E−10 | −0.662846003 | 4.19E−07 |
| | Polymerase II 2 | | | | |
| EMP1 | Epithelial Membrane Protein | −1.087294311 | 1.97E−18 | −1.304365502 | 6.10E−07 |
| | 1 | | | | |
| ENAH | ENAH Actin Regulator | −0.897620194 | 1.46E−12 | −1.082370269 | 1.27E−13 |
| ENDOD1 | Endonuclease Domain | −0.628393771 | 0.000212064 | −0.675771765 | 0.000874117 |
| | Containing 1 | | | | |
| ETV5 | ETS Variant Transcription | −0.834637861 | 0.001087421 | −0.643056597 | 2.13E−05 |
| | Factor 5 | | | | |
| FAM129B | Niban Apoptosis Regulator 2 | −0.687380122 | 3.28E−06 | −0.642971922 | 7.13E−07 |
| | (NIBAN2) | | | | |
| FAM13A | Family With Sequence | −0.803552762 | 1.44E−10 | −0.819752821 | 1.19E−15 |
| | Similarity 13 Member A | | | | |
| FAM198B | Golgi Associated Kinase | −0.557569324 | 4.52E−06 | −0.670712981 | 3.77E−06 |
| | 1B (GASK1B) | | | | |
| FAM20C | FAM20C Golgi Associated | −1.119423378 | 4.00E−08 | −1.522261971 | 3.93E−13 |
| | Secretory Pathway Kinase | | | | |
| FBLN2 | Fibulin 2 | −0.614794538 | 6.59E−05 | −1.094156756 | 5.80E−13 |
| FGF6 | Fibroblast Growth Factor 6 | −2.430454319 | 1.63E−19 | −3.074000981 | 0.001132539 |
| FHL1 | Four And A Half LIM | −1.191846477 | 2.41E−19 | −1.573994809 | 2.26E−30 |
| | Domains 1 | | | | |
| FIBIN | Fin Bud Initiation Factor | −1.277416495 | 4.05E−06 | −1.942717266 | 6.97E−31 |
| | Homolog | | | | |
| FLNC | Filamin C | −0.863625516 | 3.62E−32 | −0.938114684 | 3.05E−14 |
| FREM1 | FRAS1 Related Extracellular | −1.590135898 | 1.12E−05 | −2.077236817 | 1.07E−09 |
| | Matrix 1 | | | | |
| FRZB | Frizzled Related Protein | −1.497641314 | 8.23E−19 | −1.590421613 | 1.57E−21 |
| FSTL1 | Follistatin Like 1 | −0.544741044 | 0.000733786 | −1.076535645 | 1.05E−14 |
| FXYD5 | FXYD Domain Containing | −0.512748061 | 4.32E−05 | −0.650496179 | 0.00016607 |
| | Ion Transport Regulator 5 | | | | |
| GAS2L3 | Growth Arrest Specific 2 | −0.73615464 | 4.17E−06 | −1.299035636 | 0.0027731 |
| | Like 3 | | | | |
| GM11490 | GM11490 | −1.761115635 | 1.63E−07 | −1.559101117 | 0.00117826 |
| GM11756 | GM11756 | −0.993712497 | 2.36E−05 | −0.934207222 | 0.000164493 |
| GM13889 | GM13889 | −1.073008512 | 0.000928833 | −1.584108181 | 1.26E−05 |
| GM26920 | GM26920 | −6.173501302 | 2.65E−07 | −4.928997888 | 2.95E−05 |
| GM28438 | GM28438 | −6.180781447 | 8.31E−07 | −5.860222628 | 2.49E−06 |
| GM3776 | GM3776 | −1.926225963 | 1.09E−07 | −1.606908358 | 5.18E−06 |
| GPR153 | G Protein-Coupled Receptor | −0.773084789 | 0.001741082 | −0.773456732 | 1.23E−07 |
| | 153 | | | | |
| GRIP1 | Glutamate Receptor | −1.636027766 | 1.49E−06 | −2.410792866 | 1.45E−14 |
| | Interacting Protein 1 | | | | |
| GSTA1 | Glutathione S-Transferase | −1.349996866 | 0.000185994 | −1.59563336 | 1.24E−07 |
| | Alpha 1 | | | | |
| GSTO1 | Glutathione S-Transferase | −0.591114833 | 5.75E−05 | −0.633016078 | 3.21E−10 |
| | Omega 1 | | | | |
| HAGH | Hydroxyacylglutathione | −0.627444012 | 2.73E−08 | −0.671269585 | 1.23E−06 |
| | Hydrolase | | | | |
| HBEGF | Heparin Binding EGF Like | −1.481125243 | 1.64E−32 | −1.65985328 | 1.24E−06 |
| | Growth Factor | | | | |
| HSPA1A | Heat Shock Protein Family | −2.271125363 | 2.41E−14 | −1.549174012 | 3.55E−11 |
| | A (Hsp70) Member 1A | | | | |
| HSPA1B | Heat Shock Protein Family | −2.451140179 | 2.95E−13 | −1.828024498 | 1.87E−08 |
| | A (Hsp70) Member 1B | | | | |
| HSPA1L | Heat Shock Protein Family | −0.801745732 | 0.000110818 | −0.866134847 | 0.000703016 |
| | A (Hsp70) Member 1 Like | | | | |
| HSPB1 | Heat Shock Protein Family B | −0.617738969 | 1.80E−08 | −0.706760502 | 1.37E−07 |
| | (Small) Member 1 | | | | |
| HSPB6 | Heat Shock Protein Family B | −0.69460903 | 1.89E−07 | −0.789135702 | 6.32E−09 |
| | (Small) Member 6 | | | | |
| IFNGR1 | Interferon Gamma Receptor | −0.56418786 | 4.84E−06 | −0.620115824 | 2.04E−08 |
| | 1 | | | | |
| IGFBP7 | Insulin Like Growth Factor | −0.569709672 | 9.26E−07 | −0.894382315 | 4.78E−14 |
| | Binding Protein 7 | | | | |
| KCTD11 | Potassium Channel | −0.726507225 | 1.02E−06 | −0.684218537 | 4.29E−05 |
| | Tetramerization Domain | | | | |
| | Containing 11 | | | | |
| KLHL34 | Kelch Like Family Member | −0.874700488 | 0.000157963 | −1.358310984 | 2.04E−05 |
| | 34 | | | | |
| KREMEN1 | Kringle Containing | −0.583281394 | 4.10E−08 | −0.979459554 | 1.64E−15 |
| | Transmembrane Protein 1 | | | | |
| LAMC2 | Laminin Subunit Gamma 2 | −0.659911921 | 0.002320012 | −0.812487668 | 0.001697452 |
| LBP | Lipopolysaccharide Binding | −0.624622286 | 0.000110849 | −1.065801341 | 1.73E−14 |
| | Protein | | | | |
| LMCD1 | LIM And Cysteine Rich | −0.671661784 | 1.32E−07 | −1.039575481 | 6.40E−18 |
| | Domains 1 | | | | |
| LMOD2 | Leiomodin 2 | −0.667247903 | 1.27E−05 | −0.646527374 | 5.29E−07 |
| LOX | Lysyl Oxidase | −0.793813119 | 0.001540408 | −1.818397334 | 6.73E−08 |
| LOXL2 | Lysyl Oxidase Like 2 | −0.741006088 | 2.01E−07 | −0.839148612 | 1.76E−08 |
| LOXL3 | Lysyl Oxidase Like 3 | −0.81971171 | 1.77E−05 | −1.169710411 | 4.10E−11 |
| LTBP2 | Latent Transforming Growth | −1.615624613 | 0.001884328 | −3.270786386 | 1.71E−09 |
| | Factor Beta Binding Protein | | | | |
| | 2 | | | | |
| MAD2L1BP | MAD2L1 Binding Protein | −0.567561905 | 0.000198213 | −0.594485417 | 0.000219932 |
| MAFK | MAF BZIP Transcription | −0.56810678 | 0.001572145 | −0.969210708 | 3.07E−09 |
| | Factor K | | | | |
| MARVELD2 | MARVEL (Membrane- | −1.57088498 | 0.00021263 | −1.577664014 | 0.000768135 |
| | Associating) Domain | | | | |
| | Containing 2 | | | | |
| MED10 | Mediator Complex Subunit | −0.767406846 | 3.50E−06 | −0.72476506 | 7.71E−06 |
| | 10 | | | | |
| MEOX1 | Mesenchyme Homeobox 1 | −1.03870407 | 3.77E−13 | −1.78651494 | 8.18E−14 |
| MFAP5 | Microfibril Associated | −1.050998044 | 1.56E−08 | −1.456011705 | 1.62E−16 |
| | Protein 5 | | | | |
| MLLT11 | MLLT11 Transcription | −0.909970947 | 4.67E−08 | −1.266201461 | 1.05E−14 |
| | Factor 7 Cofactor | | | | |
| MOCOS | Molybdenum Cofactor | −0.696970552 | 0.003346267 | −0.784765463 | 0.001244891 |
| | Sulfurase | | | | |
| MRC2 | Mannose Receptor C Type 2 | −0.607055765 | 6.58E−07 | −0.765916258 | 3.90E−11 |
| MYH10 | Myosin Heavy Chain 10 | −0.558315296 | 5.33E−07 | −0.749361636 | 6.26E−11 |
| MYH7 | Myosin Heavy Chain 7 | −1.695370858 | 4.48E−06 | −1.698266041 | 5.61E−10 |
| MYOT | Myotilin | −1.43223444 | 3.90E−05 | −1.608708985 | 5.30E−06 |
| NABP1 | Nucleic Acid Binding | −0.805289174 | 2.37E−06 | −1.422986987 | 1.61E−18 |
| | Protein 1 | | | | |
| NKD2 | NKD Inhibitor Of WNT | −0.680106186 | 0.002544854 | −1.078857597 | 2.75E−05 |
| | Signaling Pathway 2 | | | | |
| NLRC3 | NLR Family CARD Domain | −1.699988786 | 1.75E−05 | −2.152077526 | 1.07E−10 |
| | Containing 3 | | | | |
| NPPB | Natriuretic Peptide B | −1.237537043 | 5.03E−13 | −1.588622727 | 0.000178054 |
| NQO1 | NAD(P)H Quinone | −0.844659407 | 1.71E−09 | −0.740057302 | 1.92E−13 |
| | Dehydrogenase 1 | | | | |
| NR4A3 | Nuclear Receptor Subfamily | −1.708592566 | 3.31E−12 | −1.843406032 | 6.12E−06 |
| | 4 Group A Member 3 | | | | |
| NRAP | Nebulin Related Anchoring | −0.720657878 | 6.27E−07 | −0.963745532 | 3.44E−11 |
| | Protein | | | | |
| NUAK1 | NUAK Family Kinase 1 | −1.008086786 | 2.05E−24 | −1.149562426 | 1.84E−18 |
| NUDT18 | Nudix Hydrolase 18 | −0.607712168 | 0.000784405 | −0.692108737 | 0.000489642 |
| NUPR1 | Nuclear Protein 1, | −0.755050195 | 0.00154487 | −0.959137748 | 3.76E−07 |
| | Transcriptional Regulator | | | | |
| OLFML3 | Olfactomedin Like 3 | −0.531187868 | 0.000548266 | 0.888164943 | 5.78E−09 |
| OTUD1 | OTU Deubiquitinase 1 | −1.334154314 | 0.000955781 | −0.997585412 | 0.000759332 |
| OTULIN | OTU Deubiquitinase With | −0.924455103 | 9.32E−11 | −0.945327376 | 4.66E−06 |
| | Linear Linkage Specificity | | | | |
| P3H2 | Prolyl 3-Hydroxylase 2 | −0.566537217 | 0.000211762 | −0.807542308 | 1.13E−06 |
| PACSIN1 | Protein Kinase C And Casein | −1.507772169 | 0.000145103 | −2.711448654 | 7.97E−07 |
| | Kinase Substrate In Neurons | | | | |
| | 1 | | | | |
| PANX1 | Pannexin 1 | −0.708422958 | 0.002436808 | −1.081656153 | 3.64E−05 |
| PDE8A | Phosphodiesterase 8A | −0.589914707 | 1.28E−05 | −0.819475204 | 4.00E−08 |
| PDLIM1 | PDZ And LIM Domain 1 | −0.533154521 | 9.05E−08 | −0.605031492 | 1.32E−09 |
| PFKP | Phosphofructokinase, | −0.589349563 | 4.65E−05 | −0.729788562 | 3.10E−09 |
| | Platelet | | | | |
| PHLDA1 | Pleckstrin Homology Like | −1.391283566 | 0.000259219 | −0.77036428 | 0.000399435 |
| | Domain Family A Member 1 | | | | |
| PLA1A | Phospholipase A1 Member | −0.958265667 | 6.27E−10 | −1.049074434 | 2.16E−13 |
| | A | | | | |
| PLCG2 | Phospholipase C Gamma 2 | −1.026413969 | 1.37E−05 | −1.17502481 | 1.58E−07 |
| PLEKHO1 | Pleckstrin Homology | −0.805978999 | 1.29E−06 | −0.827035217 | 1.07E−06 |
| | Domain Containing O1 | | | | |
| PLP2 | Proteolipid Protein 2 | −0.519697942 | 3.93E−06 | −0.644418878 | 3.61E−09 |
| PMEPA1 | Prostate Transmembrane | −0.629375556 | 5.96E−06 | −0.750697088 | 1.39E−05 |
| | Protein, Androgen Induced 1 | | | | |
| POSTN | Periostin | −1.563672565 | 5.78E−06 | −2.444815887 | 1.16E−07 |
| PPP1R3C | Protein Phosphatase 1 | −0.640212773 | 3.64E−08 | −0.838089933 | 6.87E−13 |
| | Regulatory Subunit 3C | | | | |
| PRG4 | Proteoglycan 4 | −0.783904767 | 8.85E−06 | −1.416606399 | 9.73E−10 |
| PRNP | Prion Protein | −0.628911717 | 5.50E−07 | −1.009503282 | 1.74E−13 |
| PRUNE2 | Prune Homolog 2 With BCH | −0.62619981 | 2.96E−05 | −0.794561165 | 1.27E−10 |
| | Domain | | | | |
| PTCH2 | Patched 2 | −1.116737498 | 0.001651717 | −1.389650384 | 0.001317105 |
| RAB31 | RAB31, Member RAS | −0.518927284 | 0.000236401 | −0.703212348 | 3.88E−06 |
| | Oncogene Family | | | | |
| RAI14 | Retinoic Acid Induced 14 | −0.61653507 | 0.001357195 | −0.591927102 | 0.002318664 |
| RAPGEF1 | Rap Guanine Nucleotide | −0.652334602 | 2.73E−09 | −0.68284268 | 1.61E−12 |
| | Exchange Factor 1 | | | | |
| RASL11B | RAS Like Family 11 | −1.320674401 | 1.78E−21 | −1.213428995 | 6.32E−12 |
| | Member B | | | | |
| RCAN1 | Regulator Of Calcineurin 1 | −0.832624109 | 4.48E−06 | −1.664929674 | 1.72E−09 |
| RNF115 | Ring Finger Protein 115 | −0.681934567 | 5.03E−06 | −1.009106343 | 2.01E−10 |
| RP23-354J5.3 | — | −7.506762745 | 0.002526418 | −7.417440123 | 0.002852987 |
| RP24-507K2.2 | — | −2.095419241 | 0.000645833 | −2.559556149 | 0.000485353 |
| RRP12 | Ribosomal RNA Processing | −0.534840645 | 0.001773017 | −0.870210064 | 0.000166958 |
| | 12 Homolog | | | | |
| RTN4 | Reticulon 4 | −0.818386676 | 4.80E−07 | −1.00017728 | 0.003149232 |
| SAMD5 | Sterile Alpha Motif Domain | −0.612452797 | 0.000155603 | −0.982083106 | 5.15E−14 |
| | Containing 5 | | | | |
| SEMA3B | Semaphorin 3B | −0.790137125 | 0.000374634 | −0.615386395 | 0.001681372 |
| SERPINA3N | Serpin Family A Member 3 | −1.371336445 | 1.55E−09 | −1.947204626 | 1.85E−11 |
| SERPINE1 | Serpin Family E Member 1 | −1.78339813 | 3.36E−06 | −1.308651252 | 1.44E−13 |
| SERPINF1 | Serpin Family F Member 1 | −0.68227479 | 5.31E−05 | −1.425130637 | 4.59E−27 |
| SHISA3 | Shisa Family Member 3 | −1.657810009 | 2.98E−13 | −1.996849743 | 9.76E−17 |
| SLC22A4 | Solute Carrier Family 22 | −1.548723456 | 4.29E−11 | −1.586192986 | 3.59E−09 |
| | Member 4 | | | | |
| SLC30A4 | Solute Carrier Family 30 | −0.576739999 | 0.000299451 | −0.881105881 | 2.27E−07 |
| | Member 4 | | | | |
| SLC39A6 | Solute Carrier Family 39 | −0.713898261 | 3.99E−06 | −0.878164392 | 1.12E−06 |
| | Member 6 | | | | |
| SLC6A6 | Solute Carrier Family 6 | −0.766976402 | 2.05E−09 | −0.674271244 | 7.54E−11 |
| | Member 6 | | | | |
| SLC9A3 | Solute Carrier Family 9 | −2.398500331 | 1.37E−05 | −2.328602167 | 0.000633867 |
| | Member A3 | | | | |
| SNTB1 | Syntrophin Beta 1 | −0.932756953 | 9.61E−06 | −0.966746124 | 3.27E−06 |
| SPARC | Secreted Protein Acidic And | −0.537367853 | 6.88E−06 | −0.81614873 | 3.28E−17 |
| | Cysteine Rich | | | | |
| SPATS1 | Spermatogenesis Associated | −1.432121621 | 0.002490684 | −2.384031481 | 9.86E−06 |
| | Serine Rich 1 | | | | |
| SPCS3 | Signal Peptidase Complex | −0.58368713 | 5.35E−06 | −0.686569396 | 9.91E−09 |
| | Subunit 3 | | | | |
| SPSB4 | SplA/Ryanodine Receptor | −0.925956744 | 1.27E−05 | −1.069676602 | 1.04E−05 |
| | Domain And SOCS Box | | | | |
| | Containing 4 | | | | |
| SRXN1 | Sulfiredoxin 1 | −0.882328729 | 7.42E−06 | −0.763088643 | 0.000120025 |
| SSC5D | Scavenger Receptor Cysteine | −0.964771704 | 7.17E−08 | −1.189773955 | 5.08E−07 |
| | Rich Family Member With 5 | | | | |
| | Domains | | | | |
| STAG3 | STAG3 Cohesin Complex | −1.816133667 | 1.89E−05 | −1.593633812 | 0.000508645 |
| | Component | | | | |
| SYNPO2L | Synaptopodin 2 Like | −1.23617646 | 1.08E−16 | −1.643214749 | 1.76E−23 |
| SYT12 | Synaptotagmin 12 | −0.861487006 | 3.65E−05 | −1.044563943 | 0.000275896 |
| TBX15 | T-Box Transcription Factor | −1.249519608 | 2.34E−05 | −1.89705774 | 2.96E−12 |
| | 15 | | | | |
| TGFB2 | Transforming Growth Factor | −0.799154102 | 8.41E−07 | −1.171986109 | 1.89E−05 |
| | Beta 2 | | | | |
| TGIF1 | TGFB Induced Factor | −0.905614915 | 7.85E−06 | −1.1967442 | 4.32E−07 |
| | Homeobox 1 | | | | |
| TGM2 | Transglutaminase 2 | −0.527755512 | 2.37E−09 | −0.761837536 | 7.54E−14 |
| THBS1 | Thrombospondin 1 | −0.932793606 | 3.23E−07 | −1.228807147 | 0.00407345 |
| THBS4 | Thrombospondin 4 | −2.125007503 | 1.22E−08 | −3.505613491 | 2.71E−21 |
| TLR2 | Toll Like Receptor 2 | −0.878482606 | 0.001303565 | −0.804587909 | 0.000733641 |
| TMEM200B | Transmembrane Protein | −0.978956645 | 0.000234593 | −0.881636596 | 0.005505842 |
| | 200B | | | | |
| TMEM62 | Transmembrane Protein 62 | −0.661881645 | 0.000240462 | −0.742153416 | 0.000295076 |
| TNFRSF23 | — | −0.573465399 | 0.002338424 | −0.740892543 | 0.001313381 |
| TNMD | Tenomodulin | −2.919848317 | 0.00012461 | −5.145673262 | 1.49E−10 |
| TRIM16 | Tripartite Motif Containing | −0.67039233 | 6.00E−06 | −1.079055541 | 1.32E−12 |
| | 16 | | | | |
| TSPAN9 | Tetraspanin 9 | −0.527883032 | 2.84E−07 | −0.827101636 | 6.77E−10 |
| TTLL7 | Tubulin Tyrosine Ligase | −0.771687874 | 0.000101623 | −1.025990751 | 0.000935104 |
| | Like 7 | | | | |
| TUBA1C | Tubulin Alpha 1c | −0.867485238 | 1.88E−10 | −0.633943873 | 7.85E−05 |
| TUBB2A | Tubulin Beta 2A Class IIa | −0.631833707 | 0.000132642 | −0.875880724 | 2.89E−10 |
| UCHL1 | Ubiquitin C-Terminal | −1.250230302 | 1.63E−09 | −1.43941415 | 4.54E−09 |
| | Hydrolase L1 | | | | |
| UCK2 | Uridine-Cytidine Kinase 2 | −1.227668978 | 6.42E−12 | −1.562196619 | 4.55E−16 |
| UNC5B | Unc-5 Netrin Receptor B | −0.639398328 | 4.30E−10 | −0.830846155 | 5.08E−10 |
| USP28 | Ubiquitin Specific Peptidase | −0.509019616 | 6.03E−11 | −0.613211738 | 2.59E−14 |
| | 28 | | | | |
| VCAN | Versican | −0.634684865 | 5.47E−07 | −1.163785704 | 2.00E−05 |
| VGLL2 | Vestigial Like Family | −3.736344797 | 1.03E−05 | −5.524468379 | 7.13E−08 |
| | Member 2 | | | | |
| WDFY1 | WD Repeat And FYVE | −1.305286078 | 2.02E−08 | −1.093222537 | 6.31E−06 |
| | Domain Containing 1 | | | | |
| XIRP1 | Xin Actin Binding Repeat | −1.323644936 | 6.34E−25 | −1.148111498 | 9.96E−20 |
| | Containing 1 | | | | |
| XIRP2 | Xin Actin Binding Repeat | −1.377255439 | 7.57E−07 | −1.892947901 | 3.67E−12 |
| | Containing 2 | | | | |
| ZFP423 | Zinc Finger Protein 423 | −0.596174583 | 0.002721578 | −0.6620551 | 0.001045482 |
| ZFP770 | Zinc Finger Protein 770 | −0.723324656 | 5.18E−10 | −0.627836556 | 2.97E−09 |
| ZFP784 | Zinc Finger Protein 784 | −0.921696508 | 0.000878996 | −0.907309355 | 0.000451791 |

Statistically significant downregulated genes that are common in HEP-FGF21^{-/-} and CM-FGF21^{-/-} mice with TAC compared to respective controls that were subjected to TAC.

TABLE 18
Cardiac metabolomic analysis in
HEP-FGF21^{-/-} mice 8 weeks post-TAC

| | HEP-FGF21^{-/-} TAC vs CTRL TAC (8 weeks) | |
|---|---|---|
| Metabolites | Fold-change | P value |
| Glucose | 5.475 | 0.0055451 |
| Pyruvate | 2.1303 | 0.013048 |
| Fumarate | 2.0994 | 0.0045235 |
| Citrate | 1.8572 | 0.010462 |
| O-Phosphoethanolamine | 1.7518 | 0.015086 |
| Acetate | 1.5939 | 0.034808 |
| Alanine | 1.5672 | 0.043204 |
| ATP | 1.5354 | 0.011115 |
| N,N-Dimethylglycine | 1.3431 | 0.0084885 |
| ADP | 1.3238 | 0.039196 |
| NAD+ | 1.2526 | 0.009939 |
| Creatine | 1.2319 | 0.008295 |

Cardiac metabolites that are altered significantly in HEP-FGF21^{-/-} mice compared to control mice 8 weeks post-TAC. Statistical analysis was performed by student t-test. Ctrl TAC, n=5; HEP-FGF21^{-/-} TAC, n=5.

TABLE 19
Cardiac metabolomic analysis in CM-FGF21−/−
mice 8 weeks post-TAC

CM-FGF21^{-/-} TAC
vs. CTRL TAC (8 weeks)

| Metabolites | FC | P value |
|---|---|---|
| Aspartate | 1.7393 | 0.040462 |
| Pyruvate | 1.5421 | 0.048504 |
| Fumarate | 1.5358 | 0.033539 |
| O-Phosphoethanolamine | 1.4825 | 0.011881 |
| Taurine | 0.82496 | 0.018051 |
| sn-Glycero-3-phosphocholine | 0.77812 | 0.005704 |
| Glutamate | 0.74914 | 0.024349 |
| O-Acetylcarnitine | 0.48032 | 0.013483 |

Cardiac metabolites that are altered significantly in CM-FGF21^{-/-} mice compared to control mice 8 weeks post-TAC. Statistical analysis was performed by student t-test. Ctrl TAC, n=5; CM-FGF21^{-/-} TAC, n=5.

TABLE 20
Cardiac lipidomic analysis in
HEP-FGF21^{-/-} mice 8 weeks post-TAC

| | HEP-FGF21^{-/-} TAC | | |
|---|---|---|---|
| | vs CTRL TAC (8 weeks) | | |
| Lipid Species | Fold Change | log2(FC) | p Value |
| Acetylcarnitine | 0.22135 | −2.1756 | 0.008945 |
| Cer (d18:1/16:0) | 0.58613 | −0.7707 | 7.11E−05 |
| Cer (d18:1/22:0) | 1.7885 | 0.83876 | 0.011384 |
| Cer (d18:1/23:0) | 2.9307 | 1.5513 | 0.0019175 |
| Cer (d18:1/24:0) | 3.1527 | 1.6566 | 0.00025691 |
| CL 18:1_18:1_20:4_16:0 | 1.8235 | 0.8667 | 0.03948 |
| CL 18:1_18:2_20:4_20:4 | 3.3232 | 1.7326 | 0.022579 |
| CL 20:4_20:4_20:4_20:4 | 3.6729 | 1.8769 | 0.0070019 |
| CL 22:6_18:2_18:2_18:2 | 1.8736 | 0.9058 | 0.0048001 |
| DG 34:2 | 0.51199 | −0.9658 | 0.0005922 |
| DG 36:2 | 0.53379 | −0.90564 | 0.0005039 |
| DG 38:4 | 0.40135 | −1.3171 | 2.25E−06 |
| DG 40:6 | 0.65612 | −0.60798 | 0.045931 |
| GlcCer (d18:1/24:0) | 0.64525 | −0.63207 | 0.01411 |
| LPC 19:0 | 0.62076 | −0.6879 | 0.022973 |
| PC 16:0/18:3 | 0.63306 | −0.65959 | 0.010992 |
| PC 16:1/17:1 | 0.59509 | −0.74882 | 0.0096772 |
| PC 17:1/18:2 | 0.59257 | −0.75494 | 0.014968 |
| PC 18:0/18:1 | 0.64757 | −0.62688 | 0.0029778 |
| PC 18:1/24:0 | 0.61783 | −0.69471 | 0.017122 |
| PC 34:4b | 0.62286 | −0.68303 | 0.0031276 |
| PC 36:5a | 0.59073 | −0.75943 | 0.0062511 |
| PC O-16:1/18:2b | 1.8975 | 0.9241 | 0.0079248 |
| PC 18:0/20:3b | 0.64857 | −0.62466 | 0.01156 |
| PE 16:1/18:1 | 0.5882 | −0.76562 | 0.0073668 |
| PE 16:1/18:2 | 0.63819 | −0.64795 | 0.030283 |
| PE 18:0/18:0 | 1.5656 | 0.64675 | 0.01335 |
| PE 18:0/18:2 | 0.64161 | −0.64024 | 0.012128 |
| PE 18:1/24:1 | 0.5915 | −0.75756 | 0.0052037 |
| PE 20:0/20:4 | 0.63476 | −0.65571 | 0.0061158 |
| PE 22:0/20:4 | 0.61239 | −0.70749 | 0.0023663 |
| PE 22:0/22:6 | 0.54593 | −0.87321 | 0.0016495 |
| PE O-18:0/18:2 | 1.6841 | 0.75198 | 0.0038184 |
| PG 16:1/20:4 | 0.49525 | −1.0138 | 0.010968 |
| PG 18:2/18:2 | 0.60917 | −0.71507 | 0.016503 |
| PS 18:1/24:9 | 0.39303 | −1.3473 | 0.0046613 |
| PS 18:4/22:4 | 0.5543 | −0.85126 | 0.0096883 |
| SM 17:1/24:1 | 0.63172 | −0.66264 | 9.84E−05 |
| SM d18:1/16:0(OH) | 0.58618 | −0.77058 | 0.0017595 |
| SM d18:1/25:1 | 0.65835 | −0.60308 | 0.0034789 |
| SM d18:2/24:1 | 0.6449 | −0.63285 | 0.0021193 |
| TG 62:14 | 2.2127 | 1.1458 | 0.040713 |

Cardiac lipid species that are altered in TIEP-FGF21−/− mice 8 weeks post-TAC compared to control mice with TAC. Statistical analysis was performed by student t-test. Ctrl TAC, n=5; HEP-FGF21^{-/-} TAC, n=5. Cer—Ceramides, CL—Cardiolipins, DG—Diacylglycerols (Diglycerides), GlcCer—Glucosylceramides, LPC—Lysophosphatidylcholines, PC—Phosphatidylcholines, PE—Phosphatidyl ethanol amines, PG—Phosphatidylgylcerols, PS—Phosphatidylserines, SM—Sphingomyelin, TG—Triacylglycerols (Triglycerides).

TABLE 21
Cardiac lipidomic analysis in
CM-FGF21^{-/-} mice 8 weeks post-TAC

| | CM-FGF21^{-/-} TAC | | |
|---|---|---|---|
| | vs CTRL TAC (8 weeks) | | |
| Lipid Species | Fold Change | log2(FC) | p Value |
| Acetylcarnitine | 0.16596 | −2.5911 | 0.0040817 |
| Cer (d18:1/23:0) | 1.8653 | 0.8994 | 0.035502 |
| CL 20:4_20:4_20:4_20:4 | 2.4294 | 1.2806 | 0.017784 |
| DG 38:6 | 0.61467 | −0.70212 | 0.026616 |
| LPC 20:0 | 0.5029 | −0.99166 | 0.025699 |
| LPC 20:1 | 0.63142 | −0.66332 | 0.037313 |
| PC 17:1/18:2 | 0.61856 | −0.69302 | 0.0033199 |
| PC O-18:0/20:4 | 1.7481 | 0.80582 | 0.031136 |
| PE 16:1/18:1 | 0.61998 | −0.68972 | 0.0010327 |
| PE 18:0/18:2 | 0.65599 | −0.60826 | 0.00073146 |
| PE 20:0/20:4 | 0.58752 | −0.76728 | 0.0016329 |
| PE 22:0/22:6 | 0.64774 | −0.62651 | 0.0075761 |
| PE 36:4b | 0.58576 | −0.77161 | 0.018658 |
| PE O-16:1/16:0 | 1.5195 | 0.60359 | 0.0019679 |
| PE O-18:1/18:0 | 1.5775 | 0.65766 | 0.002678 |
| PG 16:1/20:4 | 1.6476 | 0.72033 | 0.023239 |
| PG 18:1/18:2 | 1.5096 | 0.59416 | 0.012101 |
| PG 18:2/18:2 | 1.5693 | 0.65008 | 0.0056955 |
| PS 18:1/24:9 | 0.49512 | −1.0141 | 0.0051998 |
| PS 18:4/22:4 | 0.64501 | −0.63261 | 0.010817 |
| SM d18:1/18:1 | 1.5273 | 0.611 | 0.0068117 |

Cardiac lipid species that are altered in CM-FGF21^{-/-} mice 8 weeks post-TAC compared to control mice with TAC. Statistical analysis was performed by student t-test. Ctrl TAC, n=5; CM-FGF21^{-/-} TAC, n=5. Cer—Ceramides, CL—Cardiolipins, DG—Diacylglycerols (Diglycerides), LPC—Lysophosphatidylcholines, PC—Phosphatidylcholines, PE—Phosphatidylethanolamines, PG—Phosphatidylgylcerols, PS—Phosphatidylserines, SM—Sphingomyelin.

TABLE 22

| | | | | | | | M-AAV-Ctrl-HEP- | | M-AAV-FGF21- | |
|---|---|---|---|---|---|---|---|---|---|---|
| | M-AAV-Ctrl- | | M-AAV-Ctrl- | | M-AAV- | | FGF21−/− | | HEP-FGF21−/− | |
| | Sham | | TAC | | FGF21-TAC | | TAC | | TAC | |
| M-Mode | Mean | SEM | Mean | SEM | Mean | SEM | Mean | SEM | Mean | SEM |
| LVD(s) | 1.961071 | 0.055 | 2.913*** | 0.139 | 2.967*** | 0.225 | 2.325^{$$} | 0.110 | 3.178*** | 0.132 |
| LVD(d) | 3.406 | 0.061 | 3.979* | 0.120 | 4.031* | 0.192 | 3.733 | 0.084 | 4.035** | 0.109 |
| LV | 12.274 | 0.839 | 33.216* | 3.877 | 35.630** | 6.967 | 19.036^{$$} | 2.233 | 40.877*** | 3.874 |
| Vol(s) | | | | | | | | | | |
| LV | 47.847 | 2.079 | 69.670* | 4.809 | 72.567* | 8.795 | 59.680 | 3.176 | 71.914* | 4.481 |
| Vol(d) | | | | | | | | | | |
| Stroke | 35.572 | 1.502 | 36.454 | 2.430 | 36.937 | 3.254 | 40.643 | 1.569 | 31.037 | 1.105 |
| Volume | | | | | | | | | | |
| EF (%) | 74.429 | 1.169 | 52.811*** | 3.035 | 52.388**** | 4.316 | 68.531^{$$$$} | 2.321 | 43.785**** | 2.400 |
| FS (%) | 42.452 | 1.025 | 26.906**** | 1.929 | 26.862**** | 2.701 | 37.863^{$$$$} | 1.813 | 21.407**** | 1.368 |
| Cardiac | 18.173 | 0.763 | 17.602 | 1.176 | 16.183 | 1.526 | 20.647^{$} | 0.530 | 15.802 | 0.859 |
| Output | | | | | | | | | | |
| LVAW(s) | 1.664 | 0.128 | 1.632 | 0.103 | 1.881 | 0.134 | 1.951 | 0.091 | 1.618 | 0.077 |
| LVAW(d) | 1.127 | 0.118 | 1.238 | 0.058 | 1.381 | 0.200 | 1.371 | 0.107 | 1.250568 | 0.088 |
| LVPW(s) | 1.336 | 0.122 | 1.294 | 0.074 | 1.198 | 0.153 | 1.485 | 0.133 | 1.050 | 0.147 |
| LVPW(d) | 0.841 | 0.069 | 1.035 | 0.029 | 0.945 | 0.101 | 1.028 | 0.115 | 0.861 | 0.108 |
| Heart | 511.348 | 9.996 | 483.493 | 11.179 | 481.193 | 9.974 | 509.713 | 10.721 | 507.640 | 11.380 |
| Rate | | | | | | | | | | |

Echocardiography parameters for M-AAV-Ctrl-Sham, M-AAV-Ctrl-TAC, M-AAV-FGF21-TAC, M-AAV-Ctrl-THEP-FGF21−/−TAC and M-AAV-FGF21-THEP-FGF21−/−TAC 8 weeks post TAC-Mean and SEM for echocardiography parameters measured using M-mode short axis analysis in M-AAV-Ctrl-Sham, M-AAV-Ctrl-TAC, M-AAV-FGF21-TAC, M-AAV-Ctrl-THEP-FGF21−/−TAC and M-AAV-FGF21-THEP-FGF21−/−TAC 8 weeks post TAC surgery. n=6 M-AAV-Ctrl-Sham, n=6 M-AAV-Ctrl-TAC, n=6 M-AAV-FGF21-TAC, n=6 M-AAV-Ctrl-THEP-FGF21−/−TAC and n=6 M-AAV-FGF21-THEP-FGF21−/−TAC. *p<0.05, **p<0.01 ***p<0.001 ****p<0.0001 vs M-AAV-Ctrl-Sham; ^{$$}p<0.01, ^{$$$$}p<0.0001 vs M-AAV-FGF21-HEP-FGF21−/−TAC by one-way ANOVA with Tukey Multiple comparison test.

TABLE 23

| | | | | | | | M-AAV-Ctrl- | | M-AAV-FGF21 | |
|---|---|---|---|---|---|---|---|---|---|---|
| | M-AAV-Ctrl- | | | | M-AAV-FGF21- | | HEP-FGF21−/− | | HEP-FGF21−/− | |
| | Sham | | M-AAV-Ctrl-TAC | | TAC | | TAC | | TAC | |
| B-Mode | Mean | SEM | Mean | SEM | Mean | SEM | Mean | SEM | Mean | SEM |
| EDV (μl) | 30.936 | 2.1988 | 45.993 | 1.2720 | 34.858 | 1.779 | 24.716 | 1.802 | 31.416 | 1.286 |
| ESV (μl) | 10.23 | 2.817 | 32.716 | 1.341 | 19.765 | 1.995 | 8.497 | 2.638 | 22.288 | 1.361 |
| SV (μl) | 20.703 | 2.953 | 13.389* | 2.843 | 15.090 | 3.058 | 16.213 | 2.376 | 9.124** | 3.393 |
| FS (%) | 34.566 | 2.016 | 15.747*** | 2.579 | 21.913* | 4.481 | 34.418^{$$} | 2.180 | 19.771** | 2.307 |
| CO (ml/min) | 10.208 | 4.697 | 6.877 | 3.903 | 7.631 | 4.319 | 7.724 | 3.466 | 4.607** | 5.067 |
| GLS (Peak %) | 21.971 | 2.254 | 9.409** | 2.734 | 12.531 | 2.509 | 22.835^{$$} | 2.299 | 8.909** | 2.685 |
| EF (%) | 68.031 | 1.892 | 31.907*** | 1.546 | 44.288* | 1.939 | 66.138^{$$$} | 2.218 | 34.551*** | 1.629 |
| Heart | 514 | 0.945 | 519.833 | 1.4517 | 510.833 | 1.273 | 479.166 | 1.132 | 513.5 | 1.471 |
| Rate | | | | | | | | | | |

Echocardiography parameters for M-AAV-Ctrl-Sham, M-AAV-Ctrl-TAC, M-AAV-FGF21-TAC, M-AAV-Ctrl-HP-FGF21−/−TAC and M-AAV-FGF21-HP-FGF21−/−TAC 8 weeks post TAC-Mean and SEM for echocardiography parameters measured using B-Mode long axis analysis in M-AAV-Ctrl-Sham, M-AAV-Ctrl-TAC, M-AAV-FGF2-TAC, M-AAV-Ctrl-HEP-FGF21−/−TAC and M-AAV-FGF2-HEP-FGF21−/−TAC 8 weeks post TAC surgery. n=6 M-AAV-Ctrl-Sham, n=6 M-AAV-Ctrl-TAC, n=6 M-AAV-FGF2-TAC, n=6 M-AAV-Ctrl-HEP-FGF21−/−TAC and n=6 M-AAV-FGF21-HEP-FGF21−/−TAC. *p<0.05, **p<001 **p<0.001 vs M-AAV-Ctrl-Sham; $$p<0.01, $$$p<0.001 vs M-AAV-FGF21-HEP-FGF21^{-/-}TAC by one-way ANOVA with Tukey Multiple comparison test.

TABLE 24
Candidate ASO-FGF21

| ASO-FGF21 candidates | Sequence | SEQ ID |
|---|---|---|
| Gapmer ASO-FGF21-[1] | ACGGUCTGGGGATTACGGGA | 1 |
| Gapmer ASO-FGF21-[2] | GGUGACGGGGGAAAGUAGGU | 2 |
| Gapmer ASO-FGF21-[3] | GGUGAGTGAUGUCAGGGAGG | 3 |
| Gapmer ASO-FGF21-[4] | UCUGGTGGCTGAAAAUGGUA | 4 |
| Gapmer ASO-FGF21-[5] | CAGAGGGGTGTGGGTUAAGG | 5 |
| Gapmer ASO-FGF21-[6] | CAGGUAAATTTGGGTUGUGA | 6 |
| Gapmer ASO-FGF21-[7] | CUGGUATAGGAAGTTCUUGU | 7 |
| Gapmer ASO-FGF21-[8] | UCUGGTGGCGCTTCAUGUGC | 8 |
| Gapmer ASO-FGF21-[9] | UGUGGTCTGGTGGGAGCUGA | 9 |
| Gapmer ASO-FGF21-[10] | CUUCUCCATTTCACAUCUUG | 10 |

Candidate anti-sense oligonucleotides for FGF21 inhibition. Following validation, ASO-FGF21-[2](bold) was selected for in vivo applications.

TABLE 25

| | Sham | | TAC | | TAC | | TAC | |
|---|---|---|---|---|---|---|---|---|
| | ASO | | ASO | | ASO-FGF21 | | ASO-FGF21 | |
| | Scr | | Scr | | (0-8 Wks) | | (2-8 Wks) | |
| M-Mode | Mean | SEM | Mean | SEM | Mean | SEM | Mean | SEM |
| LVD(s) | 1.872 | 0.112 | 3.229**** | 0.212 | 2.016^{$$$} | 0.108 | 2.172^{$$$} | 0.161 |
| LVD(d) | 3.359 | 0.166 | 4.235** | 0.183 | 3.523^{$} | 0.140 | 3.437^{$} | 0.187 |
| LV Vol(s) | 11.342 | 1.653 | 43.364**** | 6.511 | 13.464^{$$$} | 1.836 | 16.515^{$$$} | 3.124 |
| LV Vol(d) | 47.064 | 5.800 | 81.356** | 8.326 | 52.516^{$} | 5.170 | 49.871^{$} | 6.496 |
| Stroke | 35.722 | 4.357 | 37.992 | 2.673 | 39.052 | 3.713 | 33.356 | 3.919 |
| Volume | | | | | | | | |
| EF (%) | 76.184 | 1.687 | 47.973**** | 3.720 | 74.508^{$$$$} | 2.016 | 67.487^{$$$} | 2.937 |
| FS (%) | 44.360 | 1.636 | 24.077**** | 2.274 | 42.827^{$$$} | 1.779 | 36.911^{$$} | 2.404 |
| Cardiac | 18.003 | 1.888 | 16.960 | 1.252 | 19.642 | 1.650 | 16.259 | 1.826 |
| Output | | | | | | | | |
| LV Mass | 98.476 | 7.745 | 187.278*** | 14.149 | 108.593^{$$$} | 10.580 | 123.107^{$$} | 11.567 |
| LVAW(s) | 1.581 | 0.036 | 1.833 | 0.044 | 1.688 | 0.110 | 1.624 | 0.137 |
| LVAW(d) | 1.032 | 0.039 | 1.315 | 0.060 | 1.093 | 0.094 | 1.269 | 0.121 |
| LVPW(s) | 1.415 | 0.064 | 1.302 | 0.065 | 1.378 | 0.071 | 1.415 | 0.089 |
| LVPW(d) | 0.970 | 0.084 | 1.122 | 0.059 | 0.943 | 0.080 | 0.998 | 0.093 |
| Heart Rate | 508.520 | 12.273 | 491.876 | 8.653 | 505.690 | 9.551 | 489.221 | 6.084 |

Echocardiography parameters for M-AAV-Ctrl-Sham, M-AAV-Ctrl-TAC, M-AAV-FGF21-TAC, M-AAV-Ctrl-THEP-FGF21^{-/-}TAC and M-AAV-FGF21-THEP-FGF21^{-/-}TAC 8 weeks post TAC-Mean and SEM for echocardiography parameters measured using M-Mode short axis analysis in Sham ASO-Scr, TAC ASO-Scr, TAC ASO-FGF21 (0-8 Wks), TAC ASO-FGF21 (2-8 Wks). Sham ASO-Scr, n=6; TAC ASO-Scr, n=6; TAC ASO-FGF21 (0-8 Wks), n=6; TAC ASO-FGF21 (2-8 Wks), n=6. **p<001 ***p<0.001 ****p<0.0001 vs Sham ASO Scr; ^{$}p<0.05, ^{$$}p<0.01, ^{$$$}p<0.001 vs TAC ASO Scr by one-way ANOVA with Tukey Multiple comparison test.

TABLE 26

| | Sham | | TAC | | TAC | | TAC | |
|---|---|---|---|---|---|---|---|---|
| | ASO | | ASO | | ASO-FGF21 | | ASO-FGF21 | |
| | Scr | | Scr | | (0-8 Wks) | | (2-8 Wks) | |
| B-Mode | Mean | SEM | Mean | SEM | Mean | SEM | Mean | SEM |
| EDV (μl) | 28.788 | 3.574 | 44.18 | 8.420 | 31.745 | 2.612 | 30.67 | 5.809 |
| ESV (μl) | 8.54 | 1.909 | 30.535** | 7.344 | 13.784^{$} | 1.878 | 11.425^{$} | 2.143 |
| SV (μl) | 20.245 | 1.879 | 13.592 | 2.492 | 18.791 | 1.800 | 19.225 | 3.938 |
| FS (%) | 39.01 | 2.603 | 15.868**** | 1.844 | 30.878^{$$} | 2.631 | 32.399^{$$$} | 2.129 |
| CO (ml/min) | 10.24 | 0.653 | 6.833 | 1.226 | 9.501 | 1.034 | 9.294 | 1.742 |
| GLS (Peak %) | 22.568 | 2.841 | 9.117** | 2.211 | 18.850^{$} | 0.933 | 20.885^{$$} | 2.758 |
| EF (%) | 71.93 | 3.646 | 32.048**** | 5.047 | 57.913^{$$} | 2.999 | 61.786^{$$$} | 4.708 |
| Heart Rate | 514 | 23.729 | 506.666 | 13.169 | 498.666 | 13.477 | 503 | 20.856 |

Echocardiography parameters for M-AAV-Ctrl-Sham, M-AAV-Ctrl-TAC, M-AAV-FGF21-TAC, M-AAV-Ctrl-TTP-FGF21^{-/-}TAC and M-AAV-FGF2-GTTP-FGF21^{-/-}TAC 8 weeks post TAC- Mean and SEM for echocardiography parameters measured using B-Mode long axis analysis in Sham ASO-Scr, TAC ASO-Scr, TAC ASO-FGF21 (0-8 Wks), TAC ASO-FGF21 (2-8 Wks). Sham ASO-STr, n=6; TAC ASO-Sr, n=6; TAC ASO-FGF21 (0-8 Wks), n=6; TAC ASO-FGF21 (2-8 Wks), n=6. **p<0.01, ****p<0.0001 vs Sham ASO Scr; ^{$}p<0.05, ^{$$}p<0.01, ^{$$$}p<0.001 vs TAC ASO Scr by one-way ANOVA with Tukey Multiple comparison test.

TABLE 27
Primers information

| | | FWD Primer | SEQ | | SEQ |
|---|---|---|---|---|---|
| Gene | Species | (5′- 3′) | ID | REV Primer (5′- 3′) | ID |
| Colla1 | Mus Musculus | AAATTTAAGTCTCC | 11 | CAGATGGTTAGGTTCCTTCA | 34 |
| | | CCCAAG | | | |
| Col3a1 | Mus Musculus | CTATTTAAGGCCAG | 12 | AAGAAGGGTGAGAAGAAACC | 35 |
| | | AGCAGA | | | |
| NppB | Mus Musculus | GGAGGTCACTCCCA | 13 | AGCTGTCTCTGGGCCATTTC | 36 |
| | | TCCTCT | | | |
| β-Mhc | Mus Musculus | CTTCAACCACCACA | 14 | TCTCGATGAGGTCAATGCAG | 37 |
| | | TGTTCG | | | |
| Pstn | Mus Musculus | TATACCAGGATTTC | 15 | TGTTGGCTGGTATCTTCTTT | 38 |
| | | CACAGG | | | |
| Tgfβ1 | Mus Musculus | AGCTCCACAGAGA | 16 | AGGACCTTGCTGTACTGTGT | 39 |
| | | AGAACTG | | | |
| Il-1α | Mus Musculus | GCAACGGGAAGAT | 17 | TGACAAACTTCTGCCTGACG | 40 |
| | | TCTGAAG | | | |
| Il-1β | Mus Musculus | TCACAGCAGCACAT | 18 | TGTCCTCATCCTGGAAGGTC | 41 |
| | | CAACAA | | | |
| Tnfα | Mus Musculus | ACGGCATGGATCTC | 19 | GTGGGTGAGGAGCACGTAGT | 42 |
| | | AAAGAC | | | |
| Il10 | Mus Musculus | CCTGTGTTTAAGCT | 20 | GAAAGGACACCATAGCAAAG | 43 |
| | | GTTTCC | | | |
| Klotb | Mus Musculus | TGTTCTGCTGCGAG | 21 | TTGGGTTTACCGGACTCACG | 44 |
| | | CTGTTA | | | |
| Oxt | Mus Musculus | GCTGTGCTGGACCT | 22 | GGTAGTTCTCCTCCTGGCAG | 45 |
| | | GGATAT | | | |
| Oxtr | Mus Musculus | GTCCTGTGACCTGT | 23 | GGCCTAAGTGACTCTCACAG | 46 |
| | | GTTCTT | | | |
| Oxtr | Homo Sapiens | TTGGCAGTACCTCA | 24 | TAGACATTTGGGTTGTTTCC | 47 |
| | | AAAAGT | | | |
| Fgfr1 | Mus Musculus | CAGACTGGTCTTAG | 25 | TTTTCATCATCTCCATCTCC | 48 |
| | | GCAAAC | | | |
| Fgfr1 | Homo Sapiens | CTTCAGGTCAGGAG | 26 | GCAGACCTTCATCATTTGTT | 49 |
| | | TTTGAG | | | |
| Fgfr4 | Mus Musculus | TGGAGTCTCGGAAG | 27 | TACACGGTCAAACAACGCCT | 50 |
| | | TGCATC | | | |
| Fgfr4 | Homo Sapiens | GCATCCGCTATAAC | 28 | GCAGTCTTTAGGACTTGCAC | 51 |
| | | TACCTG | | | |
| Lrrc8a | Mus Musculus | GACATTTAGAGGCA | 29 | GCCTTAGTGTCCATGTGATT | 52 |
| | | GATTGG | | | |
| Lrrc8b | Mus Musculus | GCTATAAGCGCTAC | 30 | GTCGCTGTAGTTGCTTTTCT | 53 |
| | | CAGTGT | | | |
| Piezo1 | Mus Musculus | GCATCTTTCTCAGC | 31 | TCTGCCTGTACTTCTCCTGT | 54 |
| | | CACTAC | | | |
| M36b4 | Mus Musculus | GCGACCTGGAAGTC | 32 | ATCTGCTGCATCTGCTTGG | 55 |
| | | CAACTAC | | | |
| Rps13 | Homo Sapiens | CCTTCACAGATCGG | 33 | TCAGGAGCAAGTCCCTTAGA | 56 |
| | | TGTAATCC | | | |

Primers information—Primer sequences used for qRT-PCR analyses of mouse and human samples.

TABLE 28
Antibodies information

| | | | | Catalog |
|---|---|---|---|---|
| Protein | Species | Dilution | Company | Number |
| FGF21 | Rabbit | 1:1000 | Novateinbio | ASA-B0701 |
| pAMPK^{Thr172} | Rabbit | 1:1000 | Cell Signaling | 2535S |
| AMPK | Rabbit | 1:1000 | Cell Signaling | 2532S |
| pERK1/2 | Rabbit | 1:1000 | Cell Signaling | 9101S |
| ERK | Rabbit | 1:1000 | Cell Signaling | 4695S |
| PPARα | Mouse | 1:1000 | Santa Cruz Biotech | Sc-398394 |
| Sirtuin 1 | Mouse | 1:1000 | Cell Signaling | 8469 |
| pAKT^{Ser473} | Rabbit | 1:1000 | Cell Signaling | 9271 |
| AKT | Rabbit | 1:1000 | Cell Signaling | 9272 |
| OXTR | Rabbit | 1:1000 | Proteintech | 23045-1-AP |
| β-Actin | Mouse | 1:500 | Santa Cruz Biotech | SC-47778 |

Antibodies used for Western Blot analyses of this study.

### INCORPORATION BY REFERENCE

The Sequence Listing associated with this application is filed in electronic format via EFS-Web and is hereby incorporated by reference into the specification in its entirety. The name of the text file containing the Sequence Listing is 2023099.xml. The date of creation of the Sequence Listing is Jul. 14, 2025, and the size of the file in bytes is 49 kb.

What is claimed is:

1. A method of treating a subject with a disease, the disease selected from the group consisting of hypertension, cardiac hypertrophy, cardiomyocyte enlargement, myocardial fibrosis, liver fibrosis, and cardiac fibrosis, which comprises administering to the subject an effective amount of a fibroblast growth factor (FGF) 21 inhibitor.

2. The method of claim 1 wherein the cardiac hypertrophy is left ventricular hypertrophy.

3. The method of claim 1 wherein the FGF21 inhibitor is a cardiomyocyte FGF21 inhibitor.

4. The method of claim 1 wherein the FGF21 inhibitor is a hepatocyte FGF21 inhibitor.

5. The method of claim 1 wherein the administration of inhibition of FGF21 inhibitor is cardioprotective against transverse aortic constriction induced heart failure.

6. The method of claim 5 wherein the FGF21 inhibitor is a hepatocyte FGF21 inhibitor.

7. The method of claim 1 wherein the FGF21 inhibitor further comprises a pharmaceutically acceptable excipient.


SEQUENCE LISTING (56 sequences)

SEQ ID NO: 1 - protein, 20 aa, synthetic construct
ACGGUCTGGGGATTACGGGA

SEQ ID NO: 2 - protein, 20 aa, synthetic construct
GGUGACGGGGGAAAGUAGGU

SEQ ID NO: 3 - protein, 20 aa, synthetic construct
GGUGAGTGAUGUCAGGGAGG

SEQ ID NO: 4 - protein, 20 aa, synthetic construct
UCUGGTGGCTGAAAAUGGUA

SEQ ID NO: 5 - protein, 20 aa, synthetic construct
CAGAGGGGTGTGGGTUAAGG

SEQ ID NO: 6 - protein, 20 aa, synthetic construct
CAGGUAAATTTGGGTUGUGA

SEQ ID NO: 7 - protein, 20 aa, synthetic construct
CUGGUATAGGAAGTTCUUGU

SEQ ID NO: 8 - protein, 20 aa, synthetic construct
UCUGGTGGCGCTTCAUGUGC

SEQ ID NO: 9 - protein, 20 aa, synthetic construct
UGUGGTCTGGTGGGAGCUGA

SEQ ID NO: 10 - protein, 20 aa, synthetic construct
CUUCUCCATTTCACAUCUUG

SEQ ID NO: 11 - RNA, 20 nt, Mus musculus
  source 1..20: mol_type = unassigned RNA
aaatttaagtctcccccaag

SEQ ID NO: 12 - RNA, 20 nt, Mus musculus
  source 1..20: mol_type = unassigned RNA
ctatttaaggccagagcaga

SEQ ID NO: 13 - RNA, 20 nt, Mus musculus
  source 1..20: mol_type = unassigned RNA
ggaggtcactcccatcctct

SEQ ID NO: 14 - RNA, 20 nt, Mus musculus
  source 1..20: mol_type = unassigned RNA
cttcaaccaccacatgttcg

SEQ ID NO: 15 - RNA, 20 nt, Mus musculus
  source 1..20: mol_type = unassigned RNA
tataccaggatttccacagg

SEQ ID NO: 16 - RNA, 20 nt, Mus musculus
  source 1..20: mol_type = unassigned RNA
agctccacagagaagaactg

SEQ ID NO: 17 - RNA, 20 nt, Mus musculus
  source 1..20: mol_type = unassigned RNA
gcaacgggaagattctgaag

SEQ ID NO: 18 - RNA, 20 nt, Mus musculus
  source 1..20: mol_type = unassigned RNA
tcacagcagcacatcaacaa

SEQ ID NO: 19 - RNA, 20 nt, Mus musculus
  source 1..20: mol_type = unassigned RNA
acggcatggatctcaaagac

SEQ ID NO: 20 - RNA, 20 nt, Mus musculus
  source 1..20: mol_type = unassigned RNA
cctgtgtttaagctgtttcc

SEQ ID NO: 21 - RNA, 20 nt, Mus musculus
  source 1..20: mol_type = unassigned RNA
tgttctgctgcgagctgtta

SEQ ID NO: 22 - RNA, 20 nt, Mus musculus
  source 1..20: mol_type = unassigned RNA
gctgtgctggacctggatat

SEQ ID NO: 23 - RNA, 20 nt, Mus musculus
  source 1..20: mol_type = unassigned RNA
gtcctgtgacctgtgttctt

SEQ ID NO: 24 - RNA, 20 nt, Homo sapiens
  source 1..20: mol_type = unassigned RNA
ttggcagtacctcaaaaagt

SEQ ID NO: 25 - RNA, 20 nt, Mus musculus
  source 1..20: mol_type = unassigned RNA
cagactggtcttaggcaaac

SEQ ID NO: 26 - RNA, 20 nt, Homo sapiens
  source 1..20: mol_type = unassigned RNA
cttcaggtcaggagtttgag

SEQ ID NO: 27 - RNA, 20 nt, Mus musculus
  source 1..20: mol_type = unassigned RNA
tggagtctcggaagtgcatc

SEQ ID NO: 28 - RNA, 20 nt, Homo sapiens
  source 1..20: mol_type = unassigned RNA
gcatccgctataactacctg

SEQ ID NO: 29 - RNA, 20 nt, Mus musculus
  source 1..20: mol_type = unassigned RNA
gacatttagaggcagattgg

SEQ ID NO: 30 - RNA, 20 nt, Mus musculus
  source 1..20: mol_type = unassigned RNA
gctataagcgctaccagtgt

SEQ ID NO: 31 - RNA, 20 nt, Mus musculus
  source 1..20: mol_type = unassigned RNA
gcatctttctcagccactac

SEQ ID NO: 32 - RNA, 21 nt, Mus musculus
  source 1..21: mol_type = unassigned RNA
gcgacctggaagtccaactac

SEQ ID NO: 33 - RNA, 22 nt, Homo sapiens
  source 1..22: mol_type = unassigned RNA
ccttcacagatcggtgtaatcc

SEQ ID NO: 34 - RNA, 20 nt, Mus musculus
  source 1..20: mol_type = unassigned RNA
cagatggttaggttccttca

SEQ ID NO: 35 - RNA, 20 nt, Mus musculus
  source 1..20: mol_type = unassigned RNA
aagaagggtgagaagaaacc

SEQ ID NO: 36 - RNA, 20 nt, Mus musculus
  source 1..20: mol_type = unassigned RNA
agctgtctctgggccatttc

SEQ ID NO: 37 - RNA, 20 nt, Mus musculus
  source 1..20: mol_type = unassigned RNA
tctcgatgaggtcaatgcag

SEQ ID NO: 38 - RNA, 20 nt, Mus musculus
  source 1..20: mol_type = unassigned RNA
tgttggctggtatcttcttt

SEQ ID NO: 39 - RNA, 20 nt, Mus musculus
  source 1..20: mol_type = unassigned RNA
aggaccttgctgtactgtgt

SEQ ID NO: 40 - RNA, 20 nt, Mus musculus
  source 1..20: mol_type = unassigned RNA
tgacaaacttctgcctgacg

SEQ ID NO: 41 - RNA, 20 nt, Mus musculus
  source 1..20: mol_type = unassigned RNA
tgtcctcatcctggaaggtc

SEQ ID NO: 42 - RNA, 20 nt, Mus musculus
  source 1..20: mol_type = unassigned RNA
gtgggtgaggagcacgtagt

SEQ ID NO: 43 - RNA, 20 nt, Mus musculus
  source 1..20: mol_type = unassigned RNA
gaaaggacaccatagcaaag

SEQ ID NO: 44 - RNA, 20 nt, Mus musculus
  source 1..20: mol_type = unassigned RNA
ttgggtttaccggactcacg

SEQ ID NO: 45 - RNA, 20 nt, Mus musculus
  source 1..20: mol_type = unassigned RNA
ggtagttctcctcctggcag

SEQ ID NO: 46 - RNA, 20 nt, Mus musculus
  source 1..20: mol_type = unassigned RNA
ggcctaagtgactctcacag

SEQ ID NO: 47 - RNA, 20 nt, Homo sapiens
  source 1..20: mol_type = unassigned RNA
tagacatttgggttgtttcc

SEQ ID NO: 48 - RNA, 20 nt, Mus musculus
  source 1..20: mol_type = unassigned RNA
ttttcatcatctccatctcc

SEQ ID NO: 49 - RNA, 20 nt, Homo sapiens
  source 1..20: mol_type = unassigned RNA
gcagaccttcatcatttgtt

SEQ ID NO: 50 - RNA, 20 nt, Mus musculus
  source 1..20: mol_type = unassigned RNA
tacacggtcaaacaacgcct

SEQ ID NO: 51 - RNA, 20 nt, Homo sapiens
  source 1..20: mol_type = unassigned RNA
gcagtctttaggacttgcac

SEQ ID NO: 52 - RNA, 20 nt, Mus musculus
  source 1..20: mol_type = unassigned RNA
gccttagtgtccatgtgatt

SEQ ID NO: 53 - RNA, 20 nt, Mus musculus
  source 1..20: mol_type = unassigned RNA
gtcgctgtagttgcttttct

SEQ ID NO: 54 - RNA, 20 nt, Mus musculus
  source 1..20: mol_type = unassigned RNA
tctgcctgtacttctcctgt

SEQ ID NO: 55 - RNA, 19 nt, Mus musculus
  source 1..19: mol_type = unassigned RNA
atctgctgcatctgcttgg

SEQ ID NO: 56 - RNA, 20 nt, Homo sapiens
  source 1..20: mol_type = unassigned RNA
tcaggagcaagtcccttaga